(19) United States
(12) Patent Application Publication
(10) Pub. No.: US 2022/0208306 A1
(43) Pub. Date: Jun. 30, 2022

(54) SYSTEM AND METHODS FOR IDENTIFICATION OF NON-IMMUNOGENIC EPITOPES AND DETERMINING EFFICACY OF EPITOPES IN THERAPEUTIC REGIMENS

(71) Applicant: MEMORIAL SLOAN KETTERING CANCER CENTER, New York, NY (US)

(72) Inventors: Martin Gunther KLATT, New York, NY (US); David A. SCHEINBERG, New York, NY (US)

(21) Appl. No.: 17/607,129

(22) PCT Filed: Apr. 29, 2020
(86) PCT No.: PCT/US20/30490

§ 371 Date: Oct. 28, 2021

Related U.S. Application Data
(60) Provisional application No. 62/840,391, filed on Apr. 30, 2019.

(51) Int. Cl. G16B 30/10

(52) U.S. Cl. CPC G16B 30/10; G01N 33/5008

(57) ABSTRACT

Disclosed herein is an epitope data processing system processes amino acid sequences of a plurality of epitopes determined from a plurality of peptide fragments from a subject. The epitope data processing system identifies a human leukocyte antigen ligand match (HLA-LM) of the epitope by comparing an amino acid sequence of the epitope to an amino acid sequence of at least one unmutated human leukocyte antigen (HLA) ligand, where the HLA-LM binds to at least one HLA allele. The system determines that the epitope is a potentially immunogenic epitope (PIE) based on comparison of % rank of the epitope to the % rank of the HLA-LM for the same HLA allele. The system determines unique epitope-HLA pairs, determines epitope scores, clonality scores, and responder scores for each of the PIES, and ranks the PIEs based on the respective responder scores.

### CROSS-REFERENCE OF RELATED APPLICATIONS

This application is a U.S. National Stage Application under 35 U.S.C. § 371 of International Patent Application No. PCT/US2020/030490, filed on Apr. 29, 2020, which claims the benefit of and priority to U.S. Provisional Application No. 62/840,391 filed Apr. 30, 2019 the entire disclosure of each of which is herein incorporated by reference.

### STATEMENT OF GOVERNMENT SUPPORT

This invention was made with government support under CA008748, CA023766 and CA055349 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on May 29, 2020, is named 115872-0881_SL.txt and is 50,046 bytes in size.

### FIELD OF THE DISCLOSURE

The present disclosure is generally directed to methods for processing data to determine non-immunogenic epitopes and/or determining the efficacy of specific epitopes for use in therapeutic regimens.

### BACKGROUND OF THE DISCLOSURE

Immune-based therapies, such as immune checkpoint blockade (ICB) therapy, vaccines, and T cell therapies, are becoming increasingly popular for the treatment of many diseases, such as cancer and pathogenic infections. However, a major hurdle in developing effective immune-based therapies is the identification of new epitopes on target proteins that are capable of eliciting an immune response. Only a small fraction of new epitopes elicits immune responses in vitro and in vivo making development of target-specific therapies, such as tumor-specific therapies, difficult.

### SUMMARY OF THE DISCLOSURE

In one aspect, the disclosure includes a computer-implemented method of determining the efficacy of a therapeutic regimen in a subject in need thereof. The method includes receiving, by one or more processors, from a peptide sequencing device, a plurality of peptide fragments associated with the subject. The method further includes determining, by the one or more processors, a plurality of epitopes from the plurality of peptide fragments, each epitope of the plurality of epitopes having a % rank that is less than or equal to 2.5 for at least one human leukocyte antigen (HLA) allele. The method also includes for each epitope of the plurality of epitopes, identifying, by the one or more processors, a human leukocyte antigen ligand match (HLA-LM) of the epitope by comparing an amino acid sequence of the epitope to an amino acid sequence of at least one unmutated human leukocyte antigen (HLA) ligand, wherein the HLA-LM binds to the at least one HLA allele, determining, by the one or more processors, that the epitope is a potentially immunogenic epitope (PIE) based on a comparison of the % rank of the epitope to the % rank of the HLA-LM for the same HLA allele, and determining, by the one or more processors, one or more unique epitope-HLA pairs by comparing the % rank of the PIE for a first HLA allele to the % rank of the PIE for one or more additional HLA alleles. The method further includes generating, by the one or more processors, a list of PIEs from the plurality of epitopes, the list of PIEs including epitopes from the plurality of epitopes that have been determined as a PIE. The method further includes determining, by the one or more processors, for each PIE in the list of PIEs an epitope score by adding the number of one or more unique epitope-HLA pairs associated with the PIE. The method also includes determining, by the one or more processors, a clonality score for each PIE in the list of PIEs by dividing the respective epitope score by the total number of PIEs in the list of PIEs. The method further includes determining, by the one or more processors, for each PIE in the list of PIEs, a responder score by (i) assigning points based on the respective epitope score and the respective clonality score, and (ii) adding the assigned points. The method also includes ranking, by the one or more processors, the PIEs in the list of PIEs based on the respective responder scores.

In one aspect, the disclosure includes a computer-implemented method for determining the immunogenicity of an epitope derived from a protein. The method includes receiving, by one or more processors, amino acid sequences associated with a plurality of epitopes. The method further includes, for each epitope of the plurality of epitopes: determining, by the one or more processors, from a database, a human leukocyte antigen ligand match (HLA-LM) of the epitope based on a comparison between an amino acid sequence of the epitope and amino acid sequences of one or more unmutated human leukocyte antigen (HLA) ligands, determining, by the one or more processors, that the epitope as a potentially non-immunogenic epitope (PNIE) based on a comparison between an absolute affinity or a % rank of the HLA-LM and an absolute affinity or a % rank of the epitope, respectively, and determining, by the one or more processors, that the PNIE is a non-immunogenic epitope (NIE) based on the expression site of the protein, wherein the epitope is a NIE if the protein is not expressed in an immune-privileged site. The absolute affinity of the HLA-LM is a binding affinity of the HLA-LM to a human leukocyte antigen (HLA) allele and the absolute affinity of the epitope is a predicted binding affinity of the epitope to the HLA allele. The % rank of the HLA-LM is an absolute affinity at which the HLA-LM binds to an HLA allele relative to an absolute affinity at which one or more peptides bind to the HLA allele, and the % rank of the epitope is an absolute affinity at which the epitope binds to the HLA allele relative to an absolute affinity at which one or more peptides bind to the HLA allele.

In one aspect, the disclosure includes a composition comprising a vector that includes a polynucleotide encoding an epitope listed in any of Tables 2-4, optionally wherein the vector is a bacterial plasmid.

In one aspect, the disclosure includes a computer system. The computer system including one or more processors, and a memory storing computer code instructions stored therein, the computer code instructions when executed by the one or more processors cause the computer system to: receive from a peptide sequencing device, a plurality of peptide fragments associated with the subject, and determine a plurality of epitopes from the plurality of peptide fragments, each epitope of the plurality of epitopes having a % rank that is less than or equal to 2.5 for at least one human leukocyte antigen (HLA) allele. The memory further storing computer code instructions which when executed by the one or more processors cause the computer system to: for each epitope of the plurality of epitopes, identify a human leukocyte antigen ligand match (HLA-LM) of the epitope by comparing an amino acid sequence of the epitope to an amino acid sequence of at least one unmutated human leukocyte antigen (HLA) ligand, wherein the HLA-LM binds to the at least one HLA allele, determine that the epitope is a potentially immunogenic epitope (PIE) based on a comparison of the % rank of the epitope to the % rank of the HLA-LM for the same HLA allele, and determine one or more unique epitope-HLA pairs by comparing the % rank of the PIE for a first HLA allele to the % rank of the PIE for one or more additional HLA alleles. The memory further storing computer code instructions which when executed by the one or more processors cause the computer system to: generate a list of PIEs from the plurality of epitopes, the list of PIEs including epitopes from the plurality of epitopes that have been determined as a PIE, and determine for each PIE in the list of PIEs an epitope score by adding the number of one or more unique epitope-HLA pairs associated with the PIE. The memory further storing computer code instructions which when executed by the one or more processors cause the computer system to: determine a clonality score for each PIE in the list of PIEs by dividing the respective epitope score by the total number of PIEs in the list of PIEs, determine for each PIE in the list of PIEs, a responder score by (i) assigning points based on the respective epitope score and the respective clonality score, and (ii) adding the assigned points, and rank the PIEs in the list of PIEs based on the respective responder scores.

In one aspect, the disclosure includes a computer system. The computer system including one or more processors, and a memory storing computer code instructions stored therein, the computer code instructions when executed by the one or more processors cause the computer system to: receive amino acid sequences associated with a plurality of epitopes, and for each epitope of the plurality of epitopes, determine, from a database, a human leukocyte antigen ligand match (HLA-LM) of the epitope based on a comparison between an amino acid sequence of the epitope and amino acid sequences of one or more unmutated human leukocyte antigen (HLA) ligands, determine that the epitope as a potentially non-immunogenic epitope (PNIE) based on a comparison between an absolute affinity or a % rank of the HLA-LM and an absolute affinity or a % rank of the epitope, respectively, and determine that the PNIE is a non-immunogenic epitope (NIE) based on the expression site of the protein, wherein the epitope is a NIE if the protein is not expressed in an immune-privileged site. The absolute affinity of the HLA-LM is a binding affinity of the HLA-LM to a human leukocyte antigen (HLA) allele and the absolute affinity of the epitope is a predicted binding affinity of the epitope to the HLA allele. The % rank of the HLA-LM is an absolute affinity at which the HLA-LM binds to an HLA allele relative to an absolute affinity at which one or more peptides bind to the HLA allele, and the % rank of the epitope is an absolute affinity at which the epitope binds to the HLA allele relative to an absolute affinity at which one or more peptides bind to the HLA allele. The memory further storing computer code instructions which when executed by the one or more processors cause the computer system to: generate a list of NIEs from the plurality of epitopes, the list of NIEs including the PNIEs determined to be NIEs.

In one aspect, the disclosure includes a non-transitory computer-readable medium having computer code instructions stored thereon, the computer code instructions when executed by one or more processors cause the one or more processors to: receive from a peptide sequencing device, a plurality of peptide fragments associated with the subject, and determine a plurality of epitopes from the plurality of peptide fragments, each epitope of the plurality of epitopes having a % rank that is less than or equal to 2.5 for at least one human leukocyte antigen (HLA) allele. The computer code instructions when executed by one or more processors further cause the one or more processors to: for each epitope of the plurality of epitopes, identify a human leukocyte antigen ligand match (HLA-LM) of the epitope by comparing an amino acid sequence of the epitope to an amino acid sequence of at least one unmutated human leukocyte antigen (HLA) ligand, wherein the HLA-LM binds to the at least one HLA allele, determine that the epitope is a potentially immunogenic epitope (PIE) based on a comparison of the % rank of the epitope to the % rank of the HLA-LM for the same HLA allele, and determine one or more unique epitope-HLA pairs by comparing the % rank of the PIE for a first HLA allele to the % rank of the PIE for one or more additional HLA alleles. The computer code instructions when executed by one or more processors further cause the one or more processors to: generate a list of PIEs from the plurality of epitopes, the list of PIEs including epitopes from the plurality of epitopes that have been determined as a PIE, determine for each PIE in the list of PIEs an epitope score by adding the number of one or more unique epitope-HLA pairs associated with the PIE, and determine a clonality score for each PIE in the list of PIEs by dividing the respective epitope score by the total number of PIEs in the list of PIEs. The computer code instructions when executed by one or more processors further cause the one or more processors to: determine for each PIE in the list of PIEs, a responder score by (i) assigning points based on the respective epitope score and the respective clonality score, and (ii) adding the assigned points, and rank the PIEs in the list of PIEs based on the respective responder scores.

In one aspect, the disclosure includes a non-transitory computer-readable medium having computer code instructions stored thereon, the computer code instructions when executed by one or more processors cause the one or more processors to: receive amino acid sequences associated with a plurality of epitopes, and for each epitope of the plurality of epitopes, determine, from a database, a human leukocyte antigen ligand match (HLA-LM) of the epitope based on a comparison between an amino acid sequence of the epitope and amino acid sequences of one or more unmutated human leukocyte antigen (HLA) ligands, determine that the epitope as a potentially non-immunogenic epitope (PNIE) based on a comparison between an absolute affinity or a % rank of the HLA-LM and an absolute affinity or a % rank of the epitope, respectively, and determine that the PNIE is a non-immunogenic epitope (NIE) based on the expression site of the protein, wherein the epitope is a NIE if the protein is not expressed in an immune-privileged site. The absolute affinity of the HLA-LM is a binding affinity of the HLA-LM to a human leukocyte antigen (HLA) allele and the absolute affinity of the epitope is a predicted binding affinity of the epitope to the HLA allele. The % rank of the HLA-LM is an absolute affinity at which the HLA-LM binds to an HLA allele relative to an absolute affinity at which one or more peptides bind to the HLA allele, and the % rank of the epitope is an absolute affinity at which the epitope binds to the HLA allele relative to an absolute affinity at which one or more peptides bind to the HLA allele. The computer code instructions when executed by one or more processors further cause the one or more processors to: generate a list of NIEs from the plurality of epitopes, the list of NIEs including the PNIEs determined to be NIEs.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1A is a block diagram depicting an embodiment of a network environment comprising a client device in communication with server device;

FIG. 1B is a block diagram depicting a cloud computing environment comprising client device in communication with cloud service providers;

FIGS. 1C-1D are block diagrams depicting embodiments of computing devices useful in connection with the methods and systems described herein;

FIGS. 2A-2C provide an overview and generation of mutated and unmutated HLA ligand datasets. FIG. 2A shows a schematic overview of data acquisition for mutated and unmutated HLA ligands used for prediction of neoepitope non-immunogenicity through a similarity model. Three different sources were used for unmutated HLA ligands: published data with a low false discovery rate (1%) and high peptide yields (top left), reanalysis of mass spectrometry RAW data from aforementioned publications with the Byonic software (top middle) and MS-identified HLA ligands from the IEDB database (top right, data cut-off Sep. 20, 2018). Immunogenic and non-immunogenic neoepitopes as defined by multimer or ELISpot assays were collected from 14 different studies (bottom). All HLA ligands are 9 amino acids in length and only point-mutated neoepitopes were considered. Figure discloses SEQ ID NOS 23 and 22, respectively, in order of appearance. FIG. 2B shows peptide yields for reanalysis of mass spectrometry RAW data from three publications14,19,20. For better comparison with previous studies results are shown for peptides with 8 to 12 amino acids length and after assignment to HLA alleles with netMHCpan 4.0 with a % rank cutoff of 2.0. FIG. 2C shows an Euler diagram demonstrating overlap between three sources for 9mer HLA ligands.

FIGS. 3A-3D provide characteristics of immunogenic and non-immunogenic neoepitopes. FIG. 3A shows a comparison of affinities to the HLA complexes for immunogenic and non-immunogenic HLA ligands. To avoid bias by statistical outliers in the non-immunogenic group affinity cutoff was set to 500 nM. Affinity was predicted by netMHCpan 4.0. Means+s.d. are indicated. P value was determined by two-tailed Mann-Whitney U-test. FIG. 3B shows the percentage of immunogenic neoepitopes among all neoepitopes (left) and neoepitopes where the wild-type sequence was identified by MS counterparts (right). FIG. 3C shows a pie chart representing the frequency of specific point mutations in the neoepitope dataset. HLA ligands bearing point mutations at anchor positions 2 and 9 were not included in this analysis due to limited interaction of the mutated amino acids with the TCR. Only mutations, which were identified at least five times in the neoepitope dataset were considered. FIG. 3D shows characterization of point mutations by change of volume and hydropathy of involved amino acids. Changes in hydropathy (x-axis) and volume (y-axis) were calculated based on studies of Kyte39 and Zamyatnin40. Dotted lines indicate thresholds for hydropathy and volume that define the subset of point mutations with a tendency or significantly higher chance for T cell reactivity. P values were calculated by one-tailed binomial test.

FIGS. 4A-4D provide an exemplary prediction model strategy, criteria, application and results. FIG. 4A shows a strategy to identify a non-immunogenic neoepitope in three steps: (I) Neoepitope and a non-mutated HLA ligand have to share a certain degree of similarity in the TCR recognition area: Amino acids at positions 4,5, and 8 have to be identical, at positions 6 and 7 similar physicochemical characteristics as defined by the scoring matrix in FIG. 6 are required. (II) Affinities of the neoepitope and the matching peptide to their HLA complexes need to be in a similar range: The matching ligand must score a % rank of 4.0 or lower on any of the patient's HLA alleles and its score must fall into a 5-fold range compared to the neoepitope's affinity % rank if the presenting HLA complex of neoepitope and matching HLA ligand differ. For identical HLA complexes it has to fall into a 5-fold range for absolute affinity. Green boxes indicate that described criteria were met. Double edged arrows are labeled with the fold-change in % rank scores between two HLA alleles of the neoepitope and the matching self-peptide. (III) Non-mutated matching HLA ligands derived from proteins mostly expressed at immune-privileged sites are excluded. Figure discloses SEQ ID NOS 335-336, respectively, in order of appearance. FIG. 4B shows percentages for correct prediction of non-immunogenicity of neoepitopes in training dataset and prospectively tested studies. Studies with a minimum of 15 non-immunogenic neoepitopes are shown. FIGS. 4C-4D shows performance of prediction model depicted with fractions of correct and incorrect predictions (top), absolute numbers and statistics (middle) and effect sizes (bottom). Results are shown for prospective testing only (left panel) and the complete dataset (prospective and training set combined; right panel).

FIGS. 5A-5F provide identification of subgroups with differential response to ICB through RESPONDER score. FIGS. 5A-5B show three distinct subgroups and resulting points for RESPONDER score as defined by the neoepitope score (FIG. 5A) and the clonality score (FIG. 5B). FIG. 5C shows identification of good and poor survival subgroups after ICB using RESPONDER score in a mixed cohort of NSCLC and melanoma patients. FIG. 5D shows an identical cohort as in FIG. 5C stratified by tumor mutational load. FIGS. 5E-5F show survival subgroups identified by RESPONDER score for the melanoma cohort (FIG. 5E) and the NSCLC cohort (FIG. 5F). P values were calculated by Mantel-Cox test.

FIG. 6 provides an exemplary scoring matrix for physicochemical similarity between amino acids from neoepitopes and self-peptides. Matrix for physicochemical similarity between amino acids from neoepitopes and self-peptides was defined based on studies from Kyte38, Zamyatnin39 and Pommié et al.41. Amino acids from self-peptides are depicted in 1 letter code at x-axis, neoepitope amino acids on the y-axis. The rationale for the assigned values in the scoring system is described in Example 1.

FIGS. 7A-7B show putative examples for allelic cross-tolerance of MS-identified neoepitopes. Non-immunogenic mass spectrometry identified neoepitopes from the study of Bassani-Sternberg et al.20 were matched for corresponding wild-type HLA ligands of 8 to 12 amino acids in length. All matching sequences, the original neoepitope and the wildtype sequence in the length of the neoepitope were assigned to patient's HLA alleles by netMHCpan4.0 with a % rank cutoff of 4.0. Point-mutated amino acids are depicted in orange, putative TCR recognition area in blue. FIG. 7A shows neoepitope “RPF” assigned to HLA-A*03:01 complex and matching length variant wild-type ligand assigned to B*35:03. Figure discloses SEQ ID NOS 337-339, respectively, in order of appearance. FIG. 7B shows neoepitope “RTK” assigned to HLA-A*03:01 complex and matching length variant wild-type ligand assigned to B*27:05. Figure discloses SEQ ID NOS 340-342, respectively, in order of appearance.

FIGS. 8A-8B show performance of prediction model in training datasets and for complete datasets without assumption of allelic cross tolerance. Performance of the prediction model depicted with fractions of correct and incorrect predictions (top), absolute numbers and statistics (middle) and effect sizes (bottom). FIG. 8A shows the training dataset. FIG. 8B shows the complete dataset without assuming allelic cross tolerance.

FIGS. 9A-9B show comparison of affinities between prediction subgroups. Affinities of correct and incorrect neoepitope predictions. FIG. 9A shows immunogenic neoepitopes. FIG. 9B shows non-immunogenic neoepitopes. Mean with SD is indicated. Kruskal Wallis test was used for statistical comparison.

FIGS. 10A-10C provide an exemplary explanation of different “clonality scores” and associated characteristics. Differential presentation of one neoepitope on multiple HLA complexes depending on peptide:HLA affinities. Recognition by TCR clones, clonality score, amount of neoepitope per HLA complex and associated survival are depicted for high clonality score (FIG. 10A), low clonality score (FIG. 10B), and intermediate clonality score (FIG. 10C). All neoepitopes are considered not to have matching unmutated HLA ligands. The clonality scores in these examples are only based on 1 neoepitope and do not reflect absolute values to which points can be assigned as described in the Methods section in Example 1. This example illustrates the concept of the clonality score and how it is calculated for a single neoepitope, but not in a clinical sample.

FIGS. 11A-1111 provide examples for defining good and poor responding subgroups to ICB by use of a RESPONDER score. FIG. 11A shows NSCLC subgroup with optimized thresholds for neoepitope score. NSCLC (FIG. 11B) and melanoma (FIG. 11C) subgroups with tumor mutational load as control. FIG. 11D shows NSCLC patients with undetectable PD-L1 tumor expression and never smokers stratified by RESPONDER score. FIG. 11E shows melanoma patients with NRAS mutations stratified by RESPONDER score. FIG. 11F shows NSCLC and melanoma patients from FIGS. 11D-11E merged and stratified by RESPONDER score. FIG. 11G shows melanoma patients with BRAF mutations stratified by RESPONDER score. FIG. 11H shows melanoma patients with BRAF/NRAS wild-type sequences stratified by RESPONDER score.

FIG. 12 shows example values of match scores determined for HLA ligands in various TCR recognition areas. In particular, FIG. 12 shows the match score of 4.5 determined by summing the numerical values assigned to the TCR positions 4, 5, 6, 7, and 8. FIG. 12 also shows the match scores for the particular epitope amino acid sequence and the HLA-LM amino acid sequence in relation to various HLA alleles. Figure discloses SEQ ID NOS 343-344, respectively, in order of appearance.

FIG. 13 shows a flow diagram of an example process for determining the efficacy of a therapeutic regimen in a subject.

FIG. 14 shows an epitope data structure for storing information regarding the epitopes.

FIG. 15 shows a flow diagram of an example process for determining an immunogenicity of an epitope derived from a protein.

### DETAILED DESCRIPTION

For purposes of reading the description of the various embodiments below, the following descriptions of the sections of the specification and their respective contents may be helpful:

Section A describes a network environment and computing environment which may be useful for practicing embodiments described herein.

Section B describes embodiments of systems and methods for determining immunogenicity of epitopes of proteins and determining the efficacy of a therapeutic regimen including epitopes of proteins.

### A. Computing and Network Environment

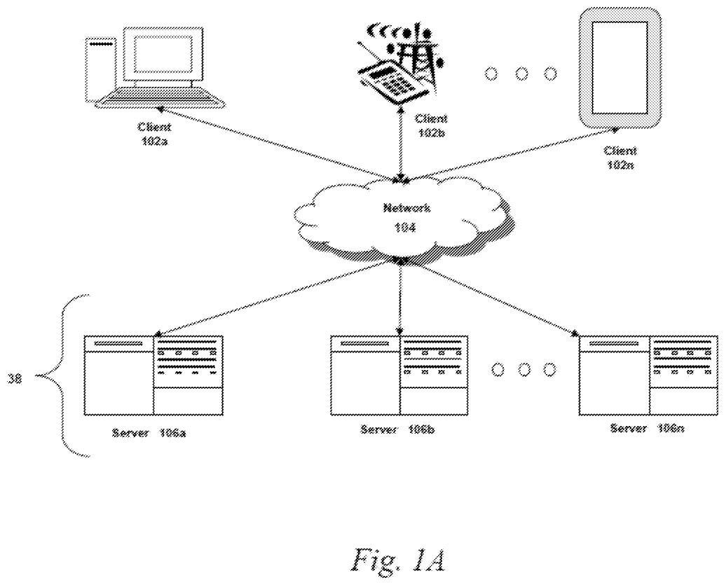

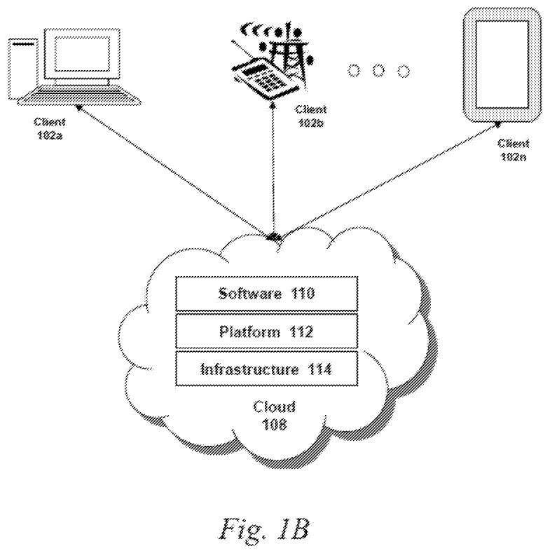

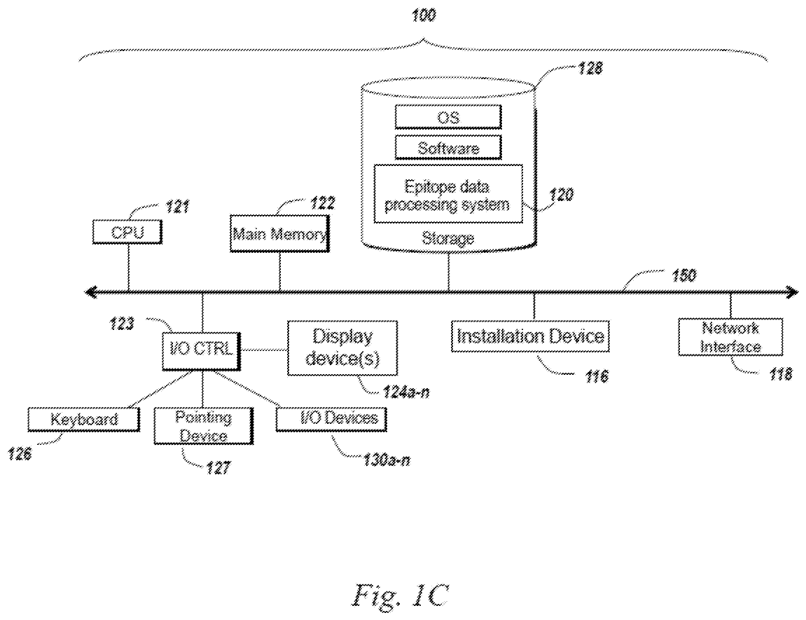

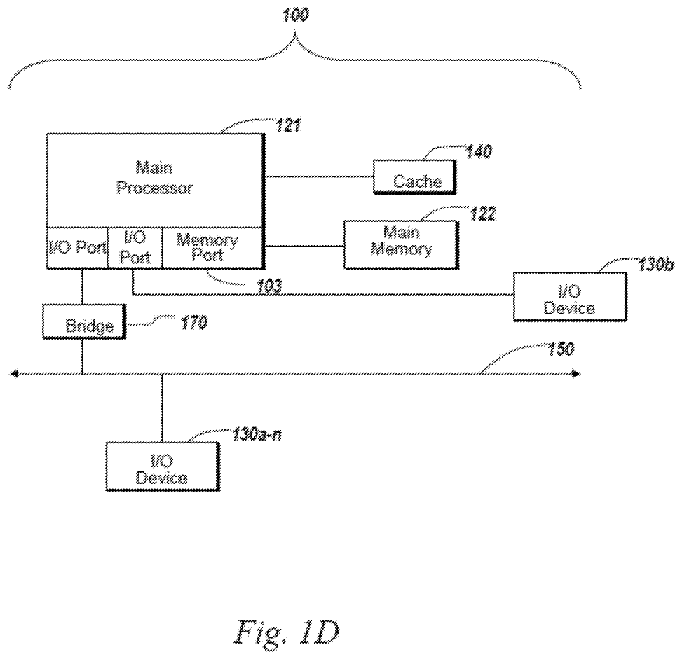

Prior to discussing specific embodiments of the present solution, it may be helpful to describe aspects of the operating environment as well as associated system components (e.g., hardware elements) in connection with the methods and systems described herein. Referring to FIG. 1A, an embodiment of a network environment is depicted. In brief overview, the network environment includes one or more clients 102a-102n (also generally referred to as local machine(s) 102, client(s) 102, client node(s) 102, client machine(s) 102, client computer(s) 102, client device(s) 102, endpoint(s) 102, or endpoint node(s) 102) in communication with one or more servers 106a-106n (also generally referred to as server(s) 106, node 106, or remote machine(s) 106) via one or more networks 104. In some embodiments, a client 102 has the capacity to function as both a client node seeking access to resources provided by a server and as a server providing access to hosted resources for other clients 102a-102n.

Although FIG. 1A shows a network 104 between the clients 102 and the servers 106, the clients 102 and the servers 106 may be on the same network 104. In some embodiments, there are multiple networks 104 between the clients 102 and the servers 106. In one of these embodiments, a network 104′ (not shown) may be a private network and a network 104 may be a public network. In another of these embodiments, a network 104 may be a private network and a network 104′ a public network. In still another of these embodiments, networks 104 and 104′ may both be private networks.

The network 104 may be connected via wired or wireless links. Wired links may include Digital Subscriber Line (DSL), coaxial cable lines, or optical fiber lines. The wireless links may include BLUETOOTH, Wi-Fi, Worldwide Interoperability for Microwave Access (WiMAX), an infrared channel or satellite band. The wireless links may also include any cellular network standards used to communicate among mobile devices, including standards that qualify as 1G, 2G, 3G, or 4G. The network standards may qualify as one or more generation of mobile telecommunication standards by fulfilling a specification or standards such as the specifications maintained by International Telecommunication Union. The 3G standards, for example, may correspond to the International Mobile Telecommunications-2000 (IMT-2000) specification, and the 4G standards may correspond to the International Mobile Telecommunications Advanced (IMT-Advanced) specification. Examples of cellular network standards include AMPS, GSM, GPRS, UMTS, LTE, LTE Advanced, Mobile WiMAX, and WiMAX-Advanced. Cellular network standards may use various channel access methods e.g. FDMA, TDMA, CDMA, or SDMA. In some embodiments, different types of data may be transmitted via different links and standards. In other embodiments, the same types of data may be transmitted via different links and standards.

The network 104 may be any type and/or form of network. The geographical scope of the network 104 may vary widely and the network 104 can be a body area network (BAN), a personal area network (PAN), a local-area network (LAN), e.g. Intranet, a metropolitan area network (MAN), a wide area network (WAN), or the Internet. The topology of the network 104 may be of any form and may include, e.g., any of the following: point-to-point, bus, star, ring, mesh, or tree. The network 104 may be an overlay network which is virtual and sits on top of one or more layers of other networks 104′. The network 104 may be of any such network topology as known to those ordinarily skilled in the art capable of supporting the operations described herein. The network 104 may utilize different techniques and layers or stacks of protocols, including, e.g., the Ethernet protocol, the internet protocol suite (TCP/IP), the ATM (Asynchronous Transfer Mode) technique, the SONET (Synchronous Optical Networking) protocol, or the SDH (Synchronous Digital Hierarchy) protocol. The TCP/IP internet protocol suite may include application layer, transport layer, internet layer (including, e.g., IPv6), or the link layer. The network 104 may be a type of a broadcast network, a telecommunications network, a data communication network, or a computer network.

In some embodiments, the system may include multiple, logically-grouped servers 106. In one of these embodiments, the logical group of servers may be referred to as a server farm 38 or a machine farm 38. In another of these embodiments, the servers 106 may be geographically dispersed. In other embodiments, a machine farm 38 may be administered as a single entity. In still other embodiments, the machine farm 38 includes a plurality of machine farms 38. The servers 106 within each machine farm 38 can be heterogeneous—one or more of the servers 106 or machines 106 can operate according to one type of operating system platform (e.g., WINDOWS NT, manufactured by Microsoft Corp. of Redmond, Wash.), while one or more of the other servers 106 can operate on according to another type of operating system platform (e.g., Unix, Linux, or Mac OS X).

In one embodiment, servers 106 in the machine farm 38 may be stored in high-density rack systems, along with associated storage systems, and located in an enterprise data center. In this embodiment, consolidating the servers 106 in this way may improve system manageability, data security, the physical security of the system, and system performance by locating servers 106 and high performance storage systems on localized high performance networks. Centralizing the servers 106 and storage systems and coupling them with advanced system management tools allows more efficient use of server resources.

The servers 106 of each machine farm 38 do not need to be physically proximate to another server 106 in the same machine farm 38. Thus, the group of servers 106 logically grouped as a machine farm 38 may be interconnected using a wide-area network (WAN) connection or a metropolitan-area network (MAN) connection. For example, a machine farm 38 may include servers 106 physically located in different continents or different regions of a continent, country, state, city, campus, or room. Data transmission speeds between servers 106 in the machine farm 38 can be increased if the servers 106 are connected using a local-area network (LAN) connection or some form of direct connection. Additionally, a heterogeneous machine farm 38 may include one or more servers 106 operating according to a type of operating system, while one or more other servers 106 execute one or more types of hypervisors rather than operating systems. In these embodiments, hypervisors may be used to emulate virtual hardware, partition physical hardware, virtualize physical hardware, and execute virtual machines that provide access to computing environments, allowing multiple operating systems to run concurrently on a host computer. Native hypervisors may run directly on the host computer. Hypervisors may include VMware ESX/ESXi, manufactured by VMWare, Inc., of Palo Alto, Calif.; the Xen hypervisor, an open source product whose development is overseen by Citrix Systems, Inc.; the HYPER-V hypervisors provided by Microsoft or others. Hosted hypervisors may run within an operating system on a second software level. Examples of hosted hypervisors may include VMware Workstation and VIRTUALBOX.

Management of the machine farm 38 may be de-centralized. For example, one or more servers 106 may comprise components, subsystems and modules to support one or more management services for the machine farm 38. In one of these embodiments, one or more servers 106 provide functionality for management of dynamic data, including techniques for handling failover, data replication, and increasing the robustness of the machine farm 38. Each server 106 may communicate with a persistent store and, in some embodiments, with a dynamic store.

Server 106 may be a file server, application server, web server, proxy server, appliance, network appliance, gateway, gateway server, virtualization server, deployment server, SSL VPN server, or firewall. In one embodiment, the server 106 may be referred to as a remote machine or a node. In another embodiment, a plurality of nodes 290 may be in the path between any two communicating servers.

Referring to FIG. 1B, a cloud computing environment is depicted. A cloud computing environment may provide client 102 with one or more resources provided by a network environment. The cloud computing environment may include one or more clients 102a-102n, in communication with the cloud 108 over one or more networks 104. Clients 102 may include, e.g., thick clients, thin clients, and zero clients. A thick client may provide at least some functionality even when disconnected from the cloud 108 or servers 106. A thin client or a zero client may depend on the connection to the cloud 108 or server 106 to provide functionality. A zero client may depend on the cloud 108 or other networks 104 or servers 106 to retrieve operating system data for the client device. The cloud 108 may include back end platforms, e.g., servers 106, storage, server farms or data centers.

The cloud 108 may be public, private, or hybrid. Public clouds may include public servers 106 that are maintained by third parties to the clients 102 or the owners of the clients. The servers 106 may be located off-site in remote geographical locations as disclosed above or otherwise. Public clouds may be connected to the servers 106 over a public network. Private clouds may include private servers 106 that are physically maintained by clients 102 or owners of clients. Private clouds may be connected to the servers 106 over a private network 104. Hybrid clouds 108 may include both the private and public networks 104 and servers 106.

The cloud 108 may also include a cloud based delivery, e.g. Software as a Service (SaaS) 110, Platform as a Service (PaaS) 112, and Infrastructure as a Service (IaaS) 114. IaaS may refer to a user renting the use of infrastructure resources that are needed during a specified time period. IaaS providers may offer storage, networking, servers or virtualization resources from large pools, allowing the users to quickly scale up by accessing more resources as needed. Examples of IaaS can include infrastructure and services (e.g., EG-32) provided by OVH HOSTING of Montreal, Quebec, Canada, AMAZON WEB SERVICES provided by Amazon.com, Inc., of Seattle, Wash., RACKSPACE CLOUD provided by Rackspace US, Inc., of San Antonio, Tex., Google Compute Engine provided by Google Inc. of Mountain View, Calif., or RIGHTSCALE provided by RightScale, Inc., of Santa Barbara, Calif. PaaS providers may offer functionality provided by IaaS, including, e.g., storage, networking, servers or virtualization, as well as additional resources such as, e.g., the operating system, middleware, or runtime resources. Examples of PaaS include WINDOWS AZURE provided by Microsoft Corporation of Redmond, Wash., Google App Engine provided by Google Inc., and HEROKU provided by Heroku, Inc. of San Francisco, Calif. SaaS providers may offer the resources that PaaS provides, including storage, networking, servers, virtualization, operating system, middleware, or runtime resources. In some embodiments, SaaS providers may offer additional resources including, e.g., data and application resources. Examples of SaaS include GOOGLE APPS provided by Google Inc., SALESFORCE provided by Salesforce.com Inc. of San Francisco, Calif., or OFFICE 365 provided by Microsoft Corporation. Examples of SaaS may also include data storage providers, e.g. DROPBOX provided by Dropbox, Inc. of San Francisco, Calif., Microsoft SKYDRIVE provided by Microsoft Corporation, Google Drive provided by Google Inc., or Apple ICLOUD provided by Apple Inc. of Cupertino, Calif.

Clients 102 may access IaaS resources with one or more IaaS standards, including, e.g., Amazon Elastic Compute Cloud (EC2), Open Cloud Computing Interface (OCCI), Cloud Infrastructure Management Interface (CIMI), or OpenStack standards. Some IaaS standards may allow clients access to resources over HTTP, and may use Representational State Transfer (REST) protocol or Simple Object Access Protocol (SOAP). Clients 102 may access PaaS resources with different PaaS interfaces. Some PaaS interfaces use HTTP packages, standard Java APIs, JavaMail API, Java Data Objects (JDO), Java Persistence API (JPA), Python APIs, web integration APIs for different programming languages including, e.g., Rack for Ruby, WSGI for Python, or PSGI for Perl, or other APIs that may be built on REST, HTTP, XML, or other protocols. Clients 102 may access SaaS resources through the use of web-based user interfaces, provided by a web browser (e.g. GOOGLE CHROME, Microsoft INTERNET EXPLORER, or Mozilla Firefox provided by Mozilla Foundation of Mountain View, Calif.). Clients 102 may also access SaaS resources through smartphone or tablet applications, including, e.g., Salesforce Sales Cloud, or Google Drive app. Clients 102 may also access SaaS resources through the client operating system, including, e.g., Windows file system for DROPBOX.

In some embodiments, access to IaaS, PaaS, or SaaS resources may be authenticated. For example, a server or authentication server may authenticate a user via security certificates, HTTPS, or API keys. API keys may include various encryption standards such as, e.g., Advanced Encryption Standard (AES). Data resources may be sent over Transport Layer Security (TLS) or Secure Sockets Layer (SSL).

The client 102 and server 106 may be deployed as and/or executed on any type and form of computing device, e.g. a computer, network device or appliance capable of communicating on any type and form of network and performing the operations described herein. FIGS. 1C-1D depict block diagrams of a computing device 100 useful for practicing an embodiment of the client 102 or a server 106. As shown in FIGS. 1C and 1D, each computing device 100 includes a central processing unit 121, and a main memory unit 122. As shown in FIG. 1C, a computing device 100 may include a storage device 128, an installation device 116, a network interface 118, an I/O controller 123, display devices 124a-124n, a keyboard 126 and a pointing device 127, e.g. a mouse. The storage device 128 may include, without limitation, an operating system, software, and a software of an epitope data processing system 120. As shown in FIG. 1D, each computing device 100 may also include additional optional elements, e.g. a memory port 103, a bridge 170, one or more input/output devices 130a-130n (generally referred to using reference numeral 130), and a cache memory 140 in communication with the central processing unit 121.

The central processing unit 121 is any logic circuitry that responds to and processes instructions fetched from the main memory unit 122. In many embodiments, the central processing unit 121 is provided by a microprocessor unit, e.g.: those manufactured by Intel Corporation of Mountain View, Calif.; those manufactured by Motorola Corporation of Schaumburg, Ill.; the ARM processor and TEGRA system on a chip (SoC) manufactured by Nvidia of Santa Clara, Calif.; the POWER7 processor, those manufactured by International Business Machines of White Plains, N.Y.; or those manufactured by Advanced Micro Devices of Sunnyvale, Calif. The computing device 100 may be based on any of these processors, or any other processor capable of operating as described herein. The central processing unit 121 may utilize instruction level parallelism, thread level parallelism, different levels of cache, and multi-core processors. A multi-core processor may include two or more processing units on a single computing component. Examples of multi-core processors include the AMD PHENOM IIX2, INTEL CORE i5 and INTEL CORE i7.

Main memory unit 122 may include one or more memory chips capable of storing data and allowing any storage location to be directly accessed by the microprocessor 121. Main memory unit 122 may be volatile and faster than storage 128 memory. Main memory units 122 may be Dynamic random access memory (DRAM) or any variants, including static random access memory (SRAM), Burst SRAM or SynchBurst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 122 or the storage 128 may be non-volatile; e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 122 may be based on any of the above described memory chips, or any other available memory chips capable of operating as described herein. In the embodiment shown in FIG. 1C, the processor 121 communicates with main memory 122 via a system bus 150 (described in more detail below). FIG. 1D depicts an embodiment of a computing device 100 in which the processor communicates directly with main memory 122 via a memory port 103. For example, in FIG. 1D the main memory 122 may be DRDRAM.

FIG. 1D depicts an embodiment in which the main processor 121 communicates directly with cache memory 140 via a secondary bus, sometimes referred to as a backside bus. In other embodiments, the main processor 121 communicates with cache memory 140 using the system bus 150. Cache memory 140 typically has a faster response time than main memory 122 and is typically provided by SRAM, BSRAM, or EDRAM. In the embodiment shown in FIG. 1D, the processor 121 communicates with various I/O devices 130 via a local system bus 150. Various buses may be used to connect the central processing unit 121 to any of the I/O devices 130, including a PCI bus, a PCI-X bus, or a PCI-Express bus, or a NuBus. For embodiments in which the I/O device is a video display 124, the processor 121 may use an Advanced Graphics Port (AGP) to communicate with the display 124 or the I/O controller 123 for the display 124. FIG. 1D depicts an embodiment of a computer 100 in which the main processor 121 communicates directly with I/O device 130b or other processors 121′ via HYPERTRANSPORT, RAPIDIO, or INFINIBAND communications technology. FIG. 1D also depicts an embodiment in which local busses and direct communication are mixed: the processor 121 communicates with I/O device 130a using a local interconnect bus while communicating with I/O device 130b directly.

A wide variety of I/O devices 130a-130n may be present in the computing device 100. Input devices may include keyboards, mice, trackpads, trackballs, touchpads, touch mice, multi-touch touchpads and touch mice, microphones, multi-array microphones, drawing tablets, cameras, single-lens reflex camera (SLR), digital SLR (DSLR), CMOS sensors, accelerometers, infrared optical sensors, pressure sensors, magnetometer sensors, angular rate sensors, depth sensors, proximity sensors, ambient light sensors, gyroscopic sensors, or other sensors. Output devices may include video displays, graphical displays, speakers, headphones, inkjet printers, laser printers, and 3D printers.

Devices 130a-130n may include a combination of multiple input or output devices, including, e.g., Microsoft KINECT, Nintendo Wiimote for the WIT, Nintendo WII U GAMEPAD, or Apple IPHONE. Some devices 130a-130n allow gesture recognition inputs through combining some of the inputs and outputs. Some devices 130a-130n provides for facial recognition which may be utilized as an input for different purposes including authentication and other commands. Some devices 130a-130n provides for voice recognition and inputs, including, e.g., Microsoft KINECT, SIRI for IPHONE by Apple, Google Now or Google Voice Search.

Additional devices 130a-130n have both input and output capabilities, including, e.g., haptic feedback devices, touchscreen displays, or multi-touch displays. Touchscreen, multi-touch displays, touchpads, touch mice, or other touch sensing devices may use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices may allow two or more contact points with the surface, allowing advanced functionality including, e.g., pinch, spread, rotate, scroll, or other gestures. Some touchscreen devices, including, e.g., Microsoft PIXELSENSE or Multi-Touch Collaboration Wall, may have larger surfaces, such as on a table-top or on a wall, and may also interact with other electronic devices. Some I/O devices 130a-130n, display devices 124a-124n or group of devices may be augment reality devices. The I/O devices may be controlled by an I/O controller 123 as shown in FIG. 1C. The I/O controller may control one or more I/O devices, such as, e.g., a keyboard 126 and a pointing device 127, e.g., a mouse or optical pen. Furthermore, an I/O device may also provide storage and/or an installation medium 116 for the computing device 100. In still other embodiments, the computing device 100 may provide USB connections (not shown) to receive handheld USB storage devices. In further embodiments, an I/O device 130 may be a bridge between the system bus 150 and an external communication bus, e.g. a USB bus, a SCSI bus, a FireWire bus, an Ethernet bus, a Gigabit Ethernet bus, a Fibre Channel bus, or a Thunderbolt bus.

In some embodiments, display devices 124a-124n may be connected to I/O controller 123. Display devices may include, e.g., liquid crystal displays (LCD), thin film transistor LCD (TFT-LCD), blue phase LCD, electronic papers (e-ink) displays, flexile displays, light emitting diode displays (LED), digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays. Examples of 3D displays may use, e.g. stereoscopy, polarization filters, active shutters, or autostereoscopy. Display devices 124a-124n may also be a head-mounted display (HMD). In some embodiments, display devices 124a-124n or the corresponding I/O controllers 123 may be controlled through or have hardware support for OPENGL or DIRECTX API or other graphics libraries.

In some embodiments, the computing device 100 may include or connect to multiple display devices 124a-124n, which each may be of the same or different type and/or form. As such, any of the I/O devices 130a-130n and/or the I/O controller 123 may include any type and/or form of suitable hardware, software, or combination of hardware and software to support, enable or provide for the connection and use of multiple display devices 124a-124n by the computing device 100. For example, the computing device 100 may include any type and/or form of video adapter, video card, driver, and/or library to interface, communicate, connect or otherwise use the display devices 124a-124n. In one embodiment, a video adapter may include multiple connectors to interface to multiple display devices 124a-124n. In other embodiments, the computing device 100 may include multiple video adapters, with each video adapter connected to one or more of the display devices 124a-124n. In some embodiments, any portion of the operating system of the computing device 100 may be configured for using multiple displays 124a-124n. In other embodiments, one or more of the display devices 124a-124n may be provided by one or more other computing devices 100a or 100b connected to the computing device 100, via the network 104. In some embodiments software may be designed and constructed to use another computer's display device as a second display device 124a for the computing device 100. For example, in one embodiment, an Apple iPad may connect to a computing device 100 and use the display of the device 100 as an additional display screen that may be used as an extended desktop. One ordinarily skilled in the art will recognize and appreciate the various ways and embodiments that a computing device 100 may be configured to have multiple display devices 124a-124n.

Referring again to FIG. 1C, the computing device 100 may comprise a storage device 128 (e.g. one or more hard disk drives or redundant arrays of independent disks) for storing an operating system or other related software, and for storing application software programs such as any program related to the software for the epitope data processing system 120. Examples of storage device 128 include, e.g., hard disk drive (HDD); optical drive including CD drive, DVD drive, or BLU-RAY drive; solid-state drive (SSD); USB flash drive; or any other device suitable for storing data. Some storage devices may include multiple volatile and non-volatile memories, including, e.g., solid state hybrid drives that combine hard disks with solid state cache. Some storage device 128 may be non-volatile, mutable, or read-only. Some storage device 128 may be internal and connect to the computing device 100 via a bus 150. Some storage devices 128 may be external and connect to the computing device 100 via an I/O device 130 that provides an external bus. Some storage device 128 may connect to the computing device 100 via the network interface 118 over a network 104, including, e.g., the Remote Disk for MACBOOK AIR by Apple. Some client devices 100 may not require a non-volatile storage device 128 and may be thin clients or zero clients 102. Some storage device 128 may also be used as an installation device 116, and may be suitable for installing software and programs. Additionally, the operating system and the software can be run from a bootable medium, for example, a bootable CD, e.g. KNOPPIX, a bootable CD for GNU/Linux that is available as a GNU/Linux distribution from knoppix.net.

Client device 100 may also install software or application from an application distribution platform. Examples of application distribution platforms include the App Store for iOS provided by Apple, Inc., the Mac App Store provided by Apple, Inc., GOOGLE PLAY for Android OS provided by Google Inc., Chrome Webstore for CHROME OS provided by Google Inc., and Amazon Appstore for Android OS and KINDLE FIRE provided by Amazon.com, Inc. An application distribution platform may facilitate installation of software on a client device 102. An application distribution platform may include a repository of applications on a server 106 or a cloud 108, which the clients 102a-102n may access over a network 104. An application distribution platform may include application developed and provided by various developers. A user of a client device 102 may select, purchase and/or download an application via the application distribution platform.

Furthermore, the computing device 100 may include a network interface 118 to interface to the network 104 through a variety of connections including, but not limited to, standard telephone lines LAN or WAN links (e.g., 802.11, T1, T3, Gigabit Ethernet, Infiniband), broadband connections (e.g., ISDN, Frame Relay, ATM, Gigabit Ethernet, Ethernet-over-SONET, ADSL, VDSL, BPON, GPON, fiber optical including FiOS), wireless connections, or some combination of any or all of the above. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMax and direct asynchronous connections). In one embodiment, the computing device 100 communicates with other computing devices 100′ via any type and/or form of gateway or tunneling protocol e.g. Secure Socket Layer (SSL) or Transport Layer Security (TLS), or the Citrix Gateway Protocol manufactured by Citrix Systems, Inc. of Ft. Lauderdale, Fla. The network interface 118 may comprise a built-in network adapter, network interface card, PCMCIA network card, EXPRESSCARD network card, card bus network adapter, wireless network adapter, USB network adapter, modem or any other device suitable for interfacing the computing device 100 to any type of network capable of communication and performing the operations described herein.

A computing device 100 of the sort depicted in FIGS. 1B-1C may operate under the control of an operating system, which controls scheduling of tasks and access to system resources. The computing device 100 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: WINDOWS 2000, WINDOWS Server 2022, WINDOWS CE, WINDOWS Phone, WINDOWS XP, WINDOWS VISTA, and WINDOWS 7, WINDOWS RT, and WINDOWS 8 all of which are manufactured by Microsoft Corporation of Redmond, Wash.; MAC OS and iOS, manufactured by Apple, Inc. of Cupertino, Calif.; and Linux, a freely-available operating system, e.g. Linux Mint distribution (“distro”) or Ubuntu, distributed by Canonical Ltd. of London, United Kingdom; or Unix or other Unix-like derivative operating systems; and Android, designed by Google, of Mountain View, Calif., among others. Some operating systems, including, e.g., the CHROME OS by Google, may be used on zero clients or thin clients, including, e.g., CHROMEBOOKS.

The computer system 100 can be any workstation, telephone, desktop computer, laptop or notebook computer, netbook, ULTRABOOK, tablet, server, handheld computer, mobile telephone, smartphone or other portable telecommunications device, media playing device, a gaming system, mobile computing device, or any other type and/or form of computing, telecommunications or media device that is capable of communication. The computer system 100 has sufficient processor power and memory capacity to perform the operations described herein. In some embodiments, the computing device 100 may have different processors, operating systems, and input devices consistent with the device. The Samsung GALAXY smartphones, e.g., operate under the control of Android operating system developed by Google, Inc. GALAXY smartphones receive input via a touch interface.

In some embodiments, the computing device 100 is a gaming system. For example, the computer system 100 may comprise a PLAYSTATION 3, or PERSONAL PLAYSTATION PORTABLE (PSP), or a PLAYSTATION VITA device manufactured by the Sony Corporation of Tokyo, Japan, a NINTENDO DS, NINTENDO 3DS, NINTENDO WII, or a NINTENDO WII U device manufactured by Nintendo Co., Ltd., of Kyoto, Japan, an XBOX 360 device manufactured by the Microsoft Corporation of Redmond, Wash.

In some embodiments, the computing device 100 is a digital audio player such as the Apple IPOD, IPOD Touch, and IPOD NANO lines of devices, manufactured by Apple Computer of Cupertino, Calif. Some digital audio players may have other functionality, including, e.g., a gaming system or any functionality made available by an application from a digital application distribution platform. For example, the IPOD Touch may access the Apple App Store. In some embodiments, the computing device 100 is a portable media player or digital audio player supporting file formats including, but not limited to, MP3, WAV, M4A/AAC, WMA Protected AAC, AIFF, Audible audiobook, Apple Lossless audio file formats and .mov, .m4v, and .mp4 MPEG-4 (H.264/MPEG-4 AVC) video file formats.

In some embodiments, the computing device 100 is a tablet e.g. the IPAD line of devices by Apple; GALAXY TAB family of devices by Samsung; or KINDLE FIRE, by Amazon.com, Inc. of Seattle, Wash. In other embodiments, the computing device 100 is an eBook reader, e.g. the KINDLE family of devices by Amazon.com, or NOOK family of devices by Barnes & Noble, Inc. of New York City, N.Y.

In some embodiments, the communications device 102 includes a combination of devices, e.g. a smartphone combined with a digital audio player or portable media player. For example, one of these embodiments is a smartphone, e.g. the IPHONE family of smartphones manufactured by Apple, Inc.; a Samsung GALAXY family of smartphones manufactured by Samsung, Inc.; or a Motorola DROID family of smartphones. In yet another embodiment, the communications device 102 is a laptop or desktop computer equipped with a web browser and a microphone and speaker system, e.g. a telephony headset. In these embodiments, the communications devices 102 are web-enabled and can receive and initiate phone calls. In some embodiments, a laptop or desktop computer is also equipped with a webcam or other video capture device that enables video chat and video call.

In some embodiments, the status of one or more machines 102, 106 in the network 104 are monitored, generally as part of network management. In one of these embodiments, the status of a machine may include an identification of load information (e.g., the number of processes on the machine, CPU and memory utilization), of port information (e.g., the number of available communication ports and the port addresses), or of session status (e.g., the duration and type of processes, and whether a process is active or idle). In another of these embodiments, this information may be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the systems and methods disclosed herein.

### B. Data Processing Methods of the Present Technology

Disclosed herein are methods and systems for determining the immunogenicity of an epitope of a protein. Generally, the methods and systems comprise determining whether an epitope has a similar sequence to a human leukocyte antigen (HLA) ligand, comparing the binding affinities of the epitope and HLA ligands for one or more HLAs, and classifying the epitope as non-immunogenic if it is not expressed in an immune-privileged site. One or more of the methods and processes discussed below can be executed by the epitope data processing system 120 discussed above in relation to FIG. 1C.

In some embodiments, the method for determining the immunogenicity of an epitope of a protein comprises: (a) identifying a human leukocyte antigen ligand match (HLA-LM) of the epitope by comparing the amino acid sequence of the epitope to the amino acid sequence of one or more human leukocyte antigen (HLA) ligands; (b) characterizing the epitope as a potentially non-immunogenic epitope (PNIE) based on a comparison of the absolute affinity or % rank score of the HLA-LM to the absolute affinity or % rank score of the epitope, wherein: (i) the absolute affinity of the HLA-LM is the binding affinity of the HLA-LM to a human leukocyte antigen (HLA), (ii) the % rank score of the HLA-LM is the absolute affinity of the HLA-LM to bind to an HLA relative to the absolute affinity of one or more peptides to bind to the HLA, (iii) the absolute affinity of the epitope is the predicted binding affinity of the epitope to a human leukocyte antigen (HLA), and (iv) the % rank score of the epitope is the absolute affinity of the epitope to bind to an HLA relative to the absolute affinity of one or more peptides to bind to the HLA; and (c) characterizing the PNIE as a non-immunogenic epitope (NIE) based on the location of expression of the protein from which the epitope is derived, wherein the epitope is a NIE if the protein is not expressed in an immune-privileged site.

Disclosed herein are methods and systems for determining the efficacy of a therapeutic regimen in a subject. Generally, the methods and systems comprise determining the immunogenicity of an epitope and calculating a responder score based on the number of unique epitope-HLA pairs and the number of immunogenic epitopes.

In some embodiments, the method for determining the efficacy of a therapeutic regimen in a subject in need thereof comprises: (a) characterizing one or more peptide fragments in the subject as an epitope if the peptide fragment has a % rank score of less than or equal to 2.5 for at least one human leukocyte antigen (HLA), wherein the % rank score of the peptide fragment is the absolute affinity of the peptide fragment to bind to an HLA relative to the absolute affinity of one or more peptides to bind to the HLA; (b) identifying a human leukocyte antigen ligand match (HLA-LM) of the epitope by comparing the amino acid sequence of the epitope to the amino acid sequence of one or more human leukocyte antigen (HLA) ligands; (c) classifying the epitope as a potentially immunogenic epitope (PIE) based on a comparison of the % rank score of the epitope to the % rank score of the HLA-LM, wherein the % rank score of the HLA-LM is the absolute affinity of the HLA-LM to bind to an HLA relative to the absolute affinity of one or more peptides to bind to the HLA; (d) identifying a unique epitope-HLA pair by comparing the % rank score of the PIE for a first HLA to the % rank score of the PIE for one or more additional HLA present in the subject; (e) calculating an epitope score by adding the number of unique epitope-HLA pairs in the subject; (f) calculating a clonality score by dividing the epitope score by the total number of PIEs in the subject; (g) calculating a responder score by (i) assigning points to the subject based on the epitope score and clonality score; and (ii) adding the assigned points; and (h) determining the efficacy of the therapeutic regimen based on the responder score. In some embodiments, upon determining that the therapeutic regimen is not effective, the method further comprises modifying the therapeutic regimen and/or administering one or more additional therapies. Modifying the therapeutic regimen may comprise increasing the dose and/or dosing frequency of the therapeutic regimen. Alternatively, modifying the therapeutic regimen comprises terminating the therapeutic regimen. In some embodiments, the subject is suffering from cancer or an infection. In some embodiments, the cancer is selected from melanoma, non-small cell lung cancer (NSCLC), cutaneous squamous skin carcinoma, small cell lung cancer (SCLC), hormone-refractory prostate cancer, triple-negative breast cancer, microsatellite instable tumor, renal cell carcinoma, urothelial carcinoma, Hodgkin's lymphoma, and Merkel cell carcinoma. In some embodiments, the infection is selected from a viral infection, bacterial infection, parasitic infection, and fungal infection. In some embodiments, the epitope is derived a protein selected from a cancer-specific protein, viral protein, bacterial protein, parasitic protein, and fungal protein. In some embodiments, the therapeutic regimen is selected from an anti-cancer therapy, anti-viral therapy, anti-bacterial therapy, anti-parasitic therapy, and anti-fungal therapy. In some embodiments, the anti-cancer therapy is an immune checkpoint blockade therapy. In some embodiments, the immune checkpoint blockade therapy is selected from an anti-PD1 therapy, anti-PDL1 therapy, and anti-CTLA4 therapy.

Disclosed herein are computer systems for performing one or more steps of the methods disclosed herein. In some embodiments, the computer system comprises: (A) one or more processors; and (B) a memory storing computer code instructions stored therein, the computer code instructions when executed by the one or more processors cause the computer system to: (i) obtain sequence information for an epitope; (ii) compare, using the sequence information, an amino acid sequence of the epitope to a plurality of amino acid sequences of a plurality of human leukocyte antigen (HLA) ligands to determine the presence or absence of one or more HLA ligand matches (HLA-LMs); (iii) compare, responsive to determining the presence of one or more HLA-LMs, an affinity or a % rank of at least one HLA-LM to a corresponding affinity or a corresponding % rank of the epitope, wherein: (a) the absolute affinity of the HLA-LM represents a binding affinity of the HLA-LM to an HLA, (b) the % rank score of the HLA-LM represents an affinity of the HLA-LM to bind to an HLA relative to the absolute affinity of one or more peptides to bind to the HLA, (c) the absolute affinity of the epitope represents a predicted binding affinity of the epitope to an HLA, and (d) the % rank score of the epitope represents an affinity of the epitope to bind to an HLA relative to the absolute affinity of one or more peptides to bind to the HLA; (iv) characterize the epitope as a potentially non-immunogenic epitope (PNIE) responsive to determining that the absolute affinity or the % rank of the HLA-LM is within a range defined based on the absolute affinity or, respectively, the percentage rank score of the epitope; and (v) identify a location of expression of a protein from which the PNIE is derived; and (vi) characterize the PNIE as a non-immunogenic epitope (NIE) when the location of expression of the protein is not an immune-privileged site.

Disclosed herein are non-transitory computer readable media (NT-CRM) having computer code instructions to perform one or more steps of the methods disclosed herein. Disclosed herein is a non-transitory computer-readable medium having computer code instructions stored thereon, wherein the computer code instructions when executed by one or more processors cause the one or more processors to: (a) obtain sequence information for an epitope; (b) compare, using the sequence information, an amino acid sequence of the epitope to a plurality of amino acid sequences of a plurality of human leukocyte antigen (HLA) ligands to determine the presence or absence of one or more HLA ligand matches (HLA-LMs); (c) compare, responsive to determining the presence of one or more HLA-LMs, an affinity or a % rank of at least one HLA-LM to a corresponding affinity or a corresponding % rank of the epitope, wherein: (i) the absolute affinity of the HLA-LM represents a binding affinity of the HLA-LM to an HLA, (ii) the % rank score of the HLA-LM represents an affinity of the HLA-LM to bind to an HLA relative to the absolute affinity of one or more peptides to bind to the HLA, (iii) the absolute affinity of the epitope represents a predicted binding affinity of the epitope to an HLA, and (iv) the % rank score of the epitope represents an affinity of the epitope to bind to an HLA relative to the absolute affinity of one or more peptides to bind to the HLA; (d) characterize the epitope as a potentially non-immunogenic epitope (PNIE) responsive to determining that the absolute affinity or the % rank of the HLA-LM is within a range defined based on the absolute affinity or, respectively, the percentage rank score of the epitope; and (e) identify a location of expression of a protein from which the PNIE is derived; and (f) characterize the PNIE as a non-immunogenic epitope (NIE) when the location of expression of the protein is not an immune-privileged site.

Identifying a Human Leukocyte Antigen Ligand Match (HLA-LM)

The methods, systems, and/or computer readable media disclosed herein may comprise identifying a human leukocyte antigen ligand match (HLA-LM) of an epitope. Identifying an HLA-LM may comprise comparing the amino acid sequence of the epitope to the amino acid sequence of one or more HLA ligands. In some embodiments, identifying an HLA-LM comprises comparing the amino acid sequence of the epitope to the amino acid sequence of 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 or more HLA ligands. In some embodiments, identifying an HLA-LM comprises comparing the amino acid sequence of the epitope to the amino acid sequence of 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, or 1000 or more HLA ligands. In some embodiments, identifying an HLA-LM comprises comparing the amino acid sequence of the epitope to the amino acid sequence of at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 or more HLA ligands. In some embodiments, identifying an HLA-LM comprises comparing the amino acid sequence of the epitope to the amino acid sequence of at least 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, or 1000 or more HLA ligands.

In some embodiments, the HLA ligands are identified from one or more databases. In some embodiments, the one or more databases are selected from genomic databases, proteomic databases, and peptidomic databases. In some embodiments, the one or more databases comprise sequencing data. In some embodiments, the HLA ligands are identified by mass spectrometry. Alternatively, or additionally, the HLA ligands are identified by non-mass spectrometric methods. In some embodiments, non-mass spectrometric methods comprise the use of one or more predictive methods or models. For instance, the predictive methods or models may predict the likelihood of a peptide being an HLA ligand. In certain embodiments, one or more predictive methods comprise inputting protein sequence data into one or more software programs that predict the likelihood of the protein sequence being an HLA ligand. In some embodiments, the protein sequence data is obtained from one or more databases containing protein sequence information. In some embodiments, the protein sequence data are obtained from the UniProt database. In some embodiments, the protein sequence data are based on human protein sequences. In certain embodiments, one or more predictive methods comprise inputting protein sequence data into one or more software programs that predicts the absolute affinity of the protein sequence to one or more HLA proteins. In certain embodiments, one or more predictive methods comprise inputting protein sequence data into one or more software programs that predicts the % rank of the protein sequence to one or more HLA proteins. In some examples, % rank can refer to the rank of the predicted affinity of a peptide (e.g., an epitope, or HLA-LM) to a MHC molecule (e.g., an HLA molecule or HLA allele) compared to a plurality (e.g., hundreds or thousands) of random natural peptides to the MHC molecule (e.g., an HLA molecule or HLA allele). This measure is not affected by inherent bias of certain molecules towards higher or lower mean predicted affinities.

In some embodiments, the software program is an MHC ligand binding prediction software program. Examples of MHC ligand binding prediction software programs include, but are not limited to, NetMHCpan 4.0, MHCflurry, SYFPEITHI, IEDB MHC-I binding predictions, RANKPEP, PREDEP, and BIMAS. In some embodiments, the software program is NetMHCpan 4.0. In some embodiments, the software program uses artificial neural networks (ANNs) to predict the likelihood of the protein sequence being an HLA ligand or the binding of the protein sequence to one or more HLA proteins. In some embodiments, the HLA is selected from HLA-A, HLA-B, HLA-C, and HLA-E. In some embodiments, the protein sequence is identified as an HLA ligand when the predicted absolute affinity to an HLA is less than or equal to 10000; 9500; 9000; 8500; 8000; 7500; 7000; 6500; 6000; 5500; 5000; 4500; 4000; 3500; 3000; 2500; 2000; 1500; 1000; 900; 800; 700; 600; or 500 nM. In some embodiments, the protein sequence is identified as an HLA ligand when the predicted absolute affinity to an HLA is less than or equal to 2000 nM. In some embodiments, the protein sequence is identified as an HLA ligand when the predicted absolute affinity to an HLA is less than or equal to 1000 nM. In some embodiments, the protein sequence is identified as an HLA ligand when the predicted absolute affinity to an HLA is less than or equal to 500 nM. In some embodiments, the protein sequence is identified as an HLA ligand when the predicted % rank for an HLA is less than or equal to 6%, 5.5%, 5%, 4.5%, 4%, 3.75%, 3.5%, 3.25%, 3%, 2.75%, 2.5%, 2.25%, 2%, 1.75%, 1.5%, 1.25%, 1%, 0.9%, 0.8%, 0.7%, 0.6%, or 0.5%. In some embodiments, the protein sequence is identified as an HLA ligand when the predicted % rank for an HLA is less than or equal to 5%. In some embodiments, the protein sequence is identified as an HLA ligand when the predicted % rank for an HLA is less than or equal to 4%. In some embodiments, the protein sequence is identified as an HLA ligand when the predicted % rank for an HLA is less than or equal to 2.5%.

In some embodiments, comparing the amino acid sequence of the epitope to the amino acid sequence of one or more HLA ligands comprises conducting a sequence alignment of the amino acid sequences.

In some embodiments, identifying an HLA-LM further comprises determining a match score for a T cell receptor (TCR) recognition area that is located within the aligned sequence between the epitope and the HLA ligand. The TCR recognition area may comprise a region of at least 1, 2, 3, 4, 5, 6, 7, 8, 9, or 10 amino acids. The TCR recognition area may comprise a region of 4 amino acids. The TCR recognition area may comprise a region of 5 amino acids. The TCR recognition area may comprise a region of 6 amino acids. The TCR recognition area may comprise a region of 7 amino acids. The TCR recognition area may comprise a region of 8 amino acids. In some embodiments, the TCR recognition area comprises consecutive amino acid residues within the epitope. In some embodiments, the TCR recognition area comprises non-consecutive amino acid residues within the epitope. In some embodiments, the TCR recognition area comprises consecutive amino acid residues within the HLA ligand. In some embodiments, the TCR recognition area comprises non-consecutive amino acid residues within the HLA ligand.

Determining the match score may comprise assigning a numerical value to one or more amino acid positions within TCR recognition area, wherein assigning a numerical value is based on the similarity of the amino acid residues at the one or more amino acid positions. The numerical value assigned to amino acid position may be based on the values provided in FIG. 6. In some embodiments, a numerical value of 1 is assigned to an amino acid position if the amino acid residue of the epitope is identical to the amino acid residue of the HLA ligand. A numerical value of 0.50 may be assigned to an amino acid position if (i) the amino acid residue of the epitope is alanine (A) and the amino acid residue of the HLA ligand is serine (S); (ii) the amino acid residue of the epitope is aspartic acid (D) and the amino acid residue of the HLA ligand is glutamic acid (E) or asparagine (N); (iii) the amino acid residue of the epitope is glutamic acid (E) and the amino acid residue of the HLA ligand is aspartic acid (D) or glutamine (Q); (iv) the amino acid residue of the epitope is phenylalanine (F) and the amino acid residue of the HLA ligand is tryptophan (W) or tyrosine (Y); (v) the amino acid residue of the epitope is glycine (G) and the amino acid residue of the HLA ligand is proline (P); (vi) the amino acid residue of the epitope is histidine (H) and the amino acid residue of the HLA ligand is glutamine (Q); (vi) the amino acid residue of the epitope is isoleucine (I) and the amino acid residue of the HLA ligand is valine (V); (vii) the amino acid residue of the epitope is lysine (K) and the amino acid residue of the HLA ligand is arginine (R); (viii) the amino acid residue of the epitope is asparagine (N) and the amino acid residue of the HLA ligand is aspartic acid (D) or glutamine (Q); (ix) the amino acid residue of the epitope is proline (P) and the amino acid residue of the HLA ligand is glycine (G); (x) the amino acid residue of the epitope is glutamine (Q) and the amino acid residue of the HLA ligand is glutamic acid (E), histidine (H), or arginine (N); (xi) the amino acid residue of the epitope is arginine (R) and the amino acid residue of the HLA ligand is lysine (K); (xii) the amino acid residue of the epitope is serine (S) and the amino acid residue of the HLA ligand is alanine (A) or threonine (T); (xiii) the amino acid residue of the epitope is threonine (T) and the amino acid residue of the HLA ligand is serine (S); (xiv) the amino acid residue of the epitope is valine (V) and the amino acid residue of the HLA ligand is isoleucine (I); (xv) the amino acid residue of the epitope is tryptophan (W) and the amino acid residue of the HLA ligand is phenylalanine (F) or tyrosine (Y); or (xvi) the amino acid residue of the epitope is tyrosine (Y) and the amino acid residue of the HLA ligand is phenylalanine (F) or tryptophan (W). A numerical value of 0.25 may be assigned to an amino acid position if (i) the amino acid residue of the epitope is phenylalanine (F) and the amino acid residue of the HLA ligand is isoleucine (I) or leucine (L); (ii) the amino acid residue of the epitope is isoleucine (I) and the amino acid residue of the HLA ligand is phenylalanine (F) or leucine (L); (iii) the amino acid residue of the epitope is leucine (L) and the amino acid residue of the HLA ligand is phenylalanine (F), isoleucine (I), methionine (M), or valine (V); (iv) the amino acid residue of the epitope is methionine (M) and the amino acid residue of the HLA ligand is leucine (L); or (v) the amino acid residue of the epitope is valine (V) and the amino acid residue of the HLA ligand is leucine (L).

In some embodiments, the match score is the sum of the numerical values assigned to the 1, 2, 3, 4, or 5 or more amino acid positions within the TCR recognition area. The match score may be the sum of the numerical values assigned to the at least 1, 2, 3, 4, or 5 or more amino acid positions within the TCR recognition area. The match score may be the numerical values assigned to the at least 1 amino acid position within the TCR recognition area. The match score may be the sum of the numerical values assigned to the at least 2 or more amino acid positions within the TCR recognition area. The match score may be the sum of the numerical values assigned to the at least 3 or more amino acid positions within the TCR recognition area. The match score may be the sum of the numerical values assigned to the at least 4 or more amino acid positions within the TCR recognition area.

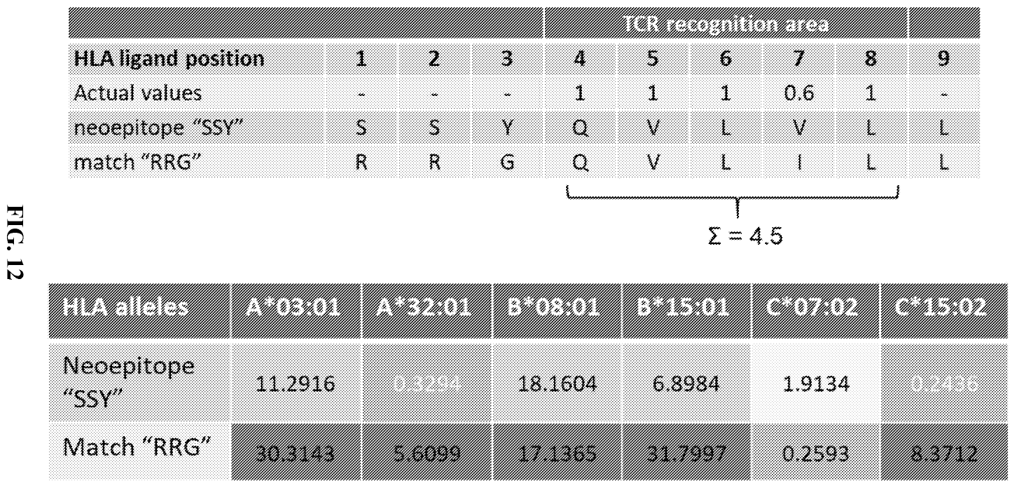

In some embodiments, the HLA ligand is identified as an HLA-LM if the match score is greater than or equal to 4. Alternatively, or additionally, the HLA ligand is identified as an HLA-LM if amino acid residues at two or more amino acid positions of the epitope are identical to amino acid residues at corresponding positions of the HLA ligand. Alternatively, or additionally, the HLA ligand is identified as an HLA-LM if amino acid residues at three or more amino acid positions of the epitope are identical to amino acid residues at corresponding positions of the HLA ligand. In some embodiments, the identical amino acid residues are located at ends of the TCR recognition area. FIG. 12 shows example values of match scores determined for HLA ligands in various TCR recognition areas. In particular, FIG. 12 shows the match score of 4.5 determined by summing the numerical values assigned to the TCR positions 4, 5, 6, 7, and 8. FIG. 12 also shows the match scores for the particular epitope amino acid sequence and the HLA-LM amino acid sequence in relation to various HLA alleles.

The amino acid sequence of an HLA ligand may be obtained from a variety of sources. For instance, the amino acid sequence of one or more HLA ligands may be obtained from one or more public databases, such as, but not limited to, the immune epitope database (IEDB), SYFPEITHI, EPIMHC, and TANTIGEN. Alternatively, or additionally, amino acid sequences of one or more HLA ligands may be obtained from datasets from published studies. Alternatively, or additionally, the amino acid sequences of one or more HLA ligands may be obtained from sequencing data from one or more subjects.

In some instances, the methods, systems, and/or computer readable media comprises obtaining mass spectra data of one or more peptides. The mass spectra data of one or more peptides may be obtained from one or more proteomic databases. Examples of proteomic databases include, but are not limited to, PRoteomics IDEntifications (PRIDE) database, MassIVE, ProteomeXchange, PeptideAtlas, iProX, jPOST, Panorama, and Proteomics DB. The methods disclosed herein may further comprise analyzing mass spectra data of one or more peptides. Mass spectra data may be analyzed using peptide and protein annotation software. Examples of peptide and protein annotation software include, but are not limited to, Byonic, Andromeda, PEAKS DB, Mascot, OMSSA, SEQUEST, Tide, MassMatrix, MS-GF+, and Protein Pilot. The methods disclosed herein may further comprise assigning one or more peptides to one or more HLA alleles. Assigning the one or more peptides to one or more HLA alleles may be based on determining the binding affinity or % rank of the one or more peptides to an HLA allele. Determining the binding affinity or % rank of the one or more peptides may comprise the use of one or more MHC analysis software programs. Examples of MHC ligand binding prediction software programs include, but are not limited to, NetMHCpan 4.0, MHCflurry, SYFPEITHI, IEDB MHC-I binding predictions, RANKPEP, PREDEP, and BIMAS. For instance, netMHCpan 4.0 may be used to determine the binding affinity or % rank of the one or more peptides.

Characterizing an Epitope as a Potentially Non-Immunogenic Epitope (PNIE)

The methods, systems, and computer readable media disclosed herein may comprise characterizing one or more epitopes as a potentially non-immunogenic epitope (PNIE). The characterization of an epitope as a PNIE may be based on a comparison of the absolute affinity of the HLA-LM for an HLA to the absolute affinity of the epitope for the same HLA. Alternatively, or additionally, characterization of an epitope as a PNIE may be based on a comparison of the absolute affinity of the HLA-LM for an HLA to the absolute affinity of the epitope for a different HLA.

In some embodiments, characterizing an epitope as a PNIE is based on a comparison of the % rank of the HLA-LM for an HLA to the % rank of the epitope for the same HLA. Alternatively, or additionally, characterizing an epitope as a PNIE is based on a comparison of the % rank of the HLA-LM for an HLA to the % rank of the epitope for a different HLA.

In some embodiments, characterizing an epitope as a PNIE is based on multiple comparisons between (i) the absolute affinity of the epitope for an HLA; and (ii) the absolute affinity of a plurality of HLA-LMs for the same HLA. Alternatively, or additionally, characterizing an epitope as a PNIE is based on multiple comparisons between (i) the absolute affinity of the epitope for an HLA; and (ii) the absolute affinity of a plurality of HLA-LMs for one or more different HLAs. Characterizing an epitope as a PNIE may be based on multiple comparisons between (i) the absolute affinity of the epitope for a plurality of HLAs; and (ii) the absolute affinity of a plurality of HLA-LMs for one or more HLAs. Characterizing an epitope as a PNIE may be based on multiple comparisons between (i) the absolute affinity of the epitope for a plurality of HLAs; and (ii) the absolute affinity of a plurality of HLA-LMs for one or more different HLAs.

In some embodiments, characterizing an epitope as a PNIE is based on multiple comparisons between (i) the % rank of the epitope for an HLA; and (ii) the % rank of a plurality of HLA-LMs for the same HLA. Alternatively, or additionally, characterizing an epitope as a PNIE is based on multiple comparisons between (i) the % rank of the epitope for an HLA; and (ii) the % rank of a plurality of HLA-LMs for one or more different HLAs. Characterizing an epitope as a PNIE may be based on multiple comparisons between (i) the % rank of the epitope for a plurality of HLAs; and (ii) the % rank of a plurality of HLA-LMs for one or more HLAs. Characterizing an epitope as a PNIE may be based on multiple comparisons between (i) the % rank of the epitope for a plurality of HLAs; and (ii) the % rank of a plurality of HLA-LMs for one or more different HLAs.

In some embodiments, the comparison of the absolute affinity is performed for 1, 2, 3, 4, 5, 6, 7, 8, 9, 10, 11, 12, 13, 14, 15, 16, 17, 18, 19, or 20 or more HLAs. In some embodiments, the comparison of the absolute affinity is performed for at least 1, 2, 3, 4, 5, 6, 7, 8, 9, 10, 11, 12, 13, 14, 15, 16, 17, 18, 19, or 20 or more HLAs. In some embodiments, the comparison of the absolute affinity is performed for 1, 2, 3, 4, 5, or 6 HLAs present in a subject. In some embodiments, the comparison of the absolute affinity is performed for at least 1, 2, 3, 4, 5, or 6 HLAs in a subject.

In some embodiments, the comparison of the % rank is performed for 1, 2, 3, 4, 5, 6, 7, 8, 9, 10, 11, 12, 13, 14, 15, 16, 17, 18, 19, or 20 or more HLAs. In some embodiments, the comparison of the % rank is performed for at least 1, 2, 3, 4, 5, 6, 7, 8, 9, 10, 11, 12, 13, 14, 15, 16, 17, 18, 19, or 20 or more HLAs. In some embodiments, the comparison of the % rank is performed for 1, 2, 3, 4, 5, or 6 HLAs present in a subject. In some embodiments, the comparison of the % rank is performed for at least 1, 2, 3, 4, 5, or 6 HLAs in a subject.

Alternatively, or additionally, the epitope is characterized as a PNIE when the absolute affinity of the HLA-LM for an HLA is within a 3, 4, 5, 6, 7, 8, 9, or 10-fold range of the absolute affinity of the epitope for the same HLA. The epitope may be characterized as a PNIE when the absolute affinity of the HLA-LM for an HLA is within a 3, 4, 5, 6, 7, 8, 9, or 10-fold range of the absolute affinity of the epitope for a different HLA. The epitope may be characterized as a PNIE when the absolute affinity of the epitope for an HLA is within a 3, 4, 5, 6, 7, 8, 9, or 10-fold range of the absolute affinity of the HLA-LM for any HLA in a subject. Alternatively, or additionally, the epitope is characterized as a PNIE when the % rank of the HLA-LM for an HLA is within a 3, 4, 5, 6, 7, 8, 9, or 10-fold range of the % rank of the epitope for the same HLA. Alternatively, or additionally, the epitope is characterized as a PNIE when the % rank of the HLA-LM for an HLA is within a 3, 4, 5, 6, 7, 8, 9, or 10-fold range of the % rank of the epitope for a different HLA. Alternatively, or additionally, the epitope is characterized as a PNIE when the absolute affinity of the HLA-LM for an HLA is within a 4-fold range of the absolute affinity of the epitope for the same HLA. The epitope may be characterized as a PNIE when the absolute affinity of the HLA-LM for an HLA is within a 4-fold range of the absolute affinity of the epitope for a different HLA. The epitope may be characterized as a PNIE when the absolute affinity of the epitope for an HLA is within a 4-fold range of the absolute affinity of the HLA-LM for any HLA in a subject. Alternatively, or additionally, the epitope is characterized as a PNIE when the % rank of the HLA-LM for an HLA is within a 4-fold range of the % rank of the epitope for the same HLA. Alternatively, or additionally, the epitope is characterized as a PNIE when the % rank of the HLA-LM for an HLA is within a 4-fold range of the % rank of the epitope for a different HLA. Alternatively, or additionally, the epitope is characterized as a PNIE when the absolute affinity of the HLA-LM for an HLA is within a 5-fold range of the absolute affinity of the epitope for the same HLA. The epitope may be characterized as a PNIE when the absolute affinity of the HLA-LM for an HLA is within a 5-fold range of the absolute affinity of the epitope for a different HLA. The epitope may be characterized as a PNIE when the absolute affinity of the epitope for an HLA is within a 5-fold range of the absolute affinity of the HLA-LM for any HLA in a subject. Alternatively, or additionally, the epitope is characterized as a PNIE when the % rank of the HLA-LM for an HLA is within a 5-fold range of the % rank of the epitope for the same HLA. Alternatively, or additionally, the epitope is characterized as a PNIE when the % rank of the HLA-LM for an HLA is within a 5-fold range of the % rank of the epitope for a different HLA. Alternatively, or additionally, the epitope is characterized as a PNIE when the absolute affinity of the HLA-LM for an HLA is within a 6-fold range of the absolute affinity of the epitope for the same HLA. The epitope may be characterized as a PNIE when the absolute affinity of the HLA-LM for an HLA is within a 6-fold range of the absolute affinity of the epitope for a different HLA. The epitope may be characterized as a PNIE when the absolute affinity of the epitope for an HLA is within a 6-fold range of the absolute affinity of the HLA-LM for any HLA in a subject. Alternatively, or additionally, the epitope is characterized as a PNIE when the % rank of the HLA-LM for an HLA is within a 6-fold range of the % rank of the epitope for the same HLA. Alternatively, or additionally, the epitope is characterized as a PNIE when the % rank of the HLA-LM for an HLA is within a 6-fold range of the % rank of the epitope for a different HLA.

Characterizing an Epitope as a Non-Immunogenic Epitope (NIE)

The methods, systems, and/or computer readable media disclosed herein may comprise characterizing an epitope as a non-immunogenic epitope (ME). Alternatively, or additionally, the methods disclosed herein may comprise characterizing a potentially non-immunogenic epitope (PNIE) as a non-immunogenic epitope (NIE). Characterizing an epitope or PNIE as a NIE may be based on the location of expression of the protein from which the epitope is derived. In some embodiments, an epitope or PNIE is characterized as a NIE when the protein from which the epitope is derived is not expressed in an immune-privileged site. In some embodiments, an epitope or PNIE is characterized as a NIE when the protein from which the epitope is derived is expressed in at least one site that is not an immune-privileged site. In some embodiments, an epitope or PNIE is characterized as a NIE when at least one protein from which the epitope is derived is expressed in at least one site that is not an immune-privileged site.

As used herein, the phrase “immune-privileged site” refers to a site in the body that is able to tolerate the introduction of antigens without eliciting an inflammatory immune response. In some embodiments, an immune-privileged site is selected from an eye, placenta, fetus, testicle, central nervous system, and hair follicle. In some embodiments, the hair follicle is an anagen hair follicle.

Characterizing an epitope or PNIE as a NIE may comprise determining the protein from which the epitope is derived. The method may comprise performing a protein alignment search to identify the protein from which the epitope is derived. In some instances, a protein basic local alignment search tool (protein BLAST) is performed to identify the protein from which the epitope is derived.

In some embodiments, the NIE is a neoepitope listed in any of Tables 2-4.

Characterizing an Epitope as a Potentially Immunogenic Epitope (PIE)

The methods, systems, and/or computer readable media disclosed herein may comprise classifying an epitope as a potentially immunogenic epitope (PIE). Classifying an epitope as a PIE may be based on a comparison of the % rank of the epitope for an HLA to the % rank of one or HLA-LMs for the HLA. Alternatively, or additionally, classifying an epitope as a PIE may be based on a comparison of the % rank of the epitope for an HLA to the % rank of one or HLA-LMs for a different HLA. Alternatively, or additionally, classifying an epitope as a PIE may be based on a comparison of the % rank of the epitope for an HLA to the % rank of one or HLA-LMs for one or more HLAs. Classifying an epitope as a PIE may be based on a comparison of the % rank of the epitope for a plurality of HLAs to the % rank of one or HLA-LMs for the corresponding HLA. Alternatively, or additionally, classifying an epitope as a PIE may be based on a comparison of the % rank of the epitope for a plurality of HLAs to the % rank of one or HLA-LMs for a plurality of different HLA.

In some embodiments, an epitope is classified as a PIE when the HLA-LM does not have a % rank of less than or equal to 10, 9.5, 9, 8.5, 8, 7.5, 7, 6.5, 6, 5.5, 5, 4.5, or 4 for at least one HLA. In some embodiments, an epitope is classified as a PIE when the HLA-LM does not have a % rank of less than or equal to 5 for at least one HLA. In some embodiments, an epitope is classified as a PIE when the HLA-LM does not have a % rank of less than or equal to 4.5 for at least one HLA. In some embodiments, an epitope is classified as a PIE when the HLA-LM does not have a % rank of less than or equal to 4 for at least one HLA. In some embodiments, an epitope is classified as a PIE when the HLA-LM does not have a % rank of less than or equal to 3.5 for at least one HLA. In some embodiments, an epitope is classified as a PIE when the HLA-LM does not have a % rank of less than or equal to 3 for at least one HLA.

Alternatively, or additionally, an epitope is classified as a PIE when the % rank of the HLA-LM is not within a 10, 9.5, 9, 8.5, 8, 7.5, 7, 6.5, 6, 5.5, 5, 4.5, 4, 3.5, 3, 2.5, or 2-fold range of the % rank of the epitope for at least one HLA. In some embodiments, an epitope is classified as a PIE when the % rank of the HLA-LM is not within a 6-fold range of the % rank of the epitope for at least one HLA. In some embodiments, an epitope is classified as a PIE when the % rank of the HLA-LM is not within a 5.5-fold range of the % rank of the epitope for at least one HLA. In some embodiments, an epitope is classified as a PIE when the % rank of the HLA-LM is not within a 5-fold range of the % rank of the epitope for at least one HLA. In some embodiments, an epitope is classified as a PIE when the % rank of the HLA-LM is not within a 4.5-fold range of the % rank of the epitope for at least one HLA. In some embodiments, an epitope is classified as a PIE when the % rank of the HLA-LM is not within a 4-fold range of the % rank of the epitope for at least one HLA.

Unique Epitope-HLA Pairs, Clonality Score, Epitope Score, Responder Score

The methods, systems, and/or computer readable media disclosed herein may comprise determining the presence or absence of one or more unique epitope-HLA pairs. The methods, systems, and/or computer readable media disclosed herein may further comprise identifying unique epitope-HLA pairs. In some embodiments, determining the presence or absence of or identifying a unique epitope-HLA pair comprises comparing the % rank of the PIE for a first HLA to the % rank of the PIE for a second HLA. Alternatively, or additionally, determining the presence or absence of or identifying a unique epitope-HLA pair comprises comparing the % rank of the PIE for a first HLA to the % rank of the PIA for one or more additional HLAs.

Alternatively, or additionally, determining the presence or absence of or identifying a unique epitope-HLA pair comprises comparing the % rank of one or more additional PIEs for an HLA to the % rank of the corresponding PIE for one or more additional HLAs. For instance, two or more epitopes may be characterized as PIEs and determining the presence or absence of or identifying a unique epitope-HLA pair may be performed for each PIE.

In some embodiments, a unique epitope-HLA pair is identified when the % rank score of the PIE for a first HLA is not within a 10, 9.5, 9, 8.5, 8, 7.5, 7, 6.5, 6, 5.5, 5, 4.5, 4, 3.5, 3, 2.5, or 2-fold range of the % rank score of the PIE for at least one additional HLA. A unique epitope-HLA pair may be identified when the % rank score of the PIE for a first HLA is not within a 6-fold range of the % rank score of the PIE for at least one additional HLAs. A unique epitope-HLA pair may be identified when the % rank score of the PIE for a first HLA is not within a 5.5-fold range of the % rank score of the PIE for at least one additional HLAs. A unique epitope-HLA pair may be identified when the % rank score of the PIE for a first HLA is not within a 5-fold range of the % rank score of the PIE for at least one additional HLAs. A unique epitope-HLA pair may be identified when the % rank score of the PIE for a first HLA is not within a 4.5-fold range of the % rank score of the PIE for at least one additional HLAs. A unique epitope-HLA pair may be identified when the % rank score of the PIE for a first HLA is not within a 4-fold range of the % rank score of the PIE for at least one additional HLAs.

In some embodiments, an epitope score is calculated based on the number of unique epitope-HLA pairs. The epitope score may be calculated by adding the number of unique epitope-HLA pairs in a subject.

In some embodiments, a clonality score is calculated based on the epitope score. The clonality score may be calculated by dividing the epitope score by the total number of PIEs.

In some embodiments, a responder score is calculated based on the epitope score and clonality score. The responder score may be calculated by assigning points based on the epitope score and/or clonality score. In some embodiments, 6 points are assigned when the epitope score is greater than 200. In some embodiments, 4 points are assigned when the epitope score is greater than 50 and less than 200. In some embodiments, 2 points are assigned when the epitope score is less than or equal to 50.

Alternatively, or additionally, 3 points are assigned when the clonality score is greater than 0.7 and less than or equal to 0.84. In some embodiments, 2 points when the clonality score is less than or equal to 7. In some embodiments, 1 point is assigned when the clonality score is greater than 0.84.

In some embodiments, the responder score is calculated by adding the assigned points based on the epitope score and clonality score. In some embodiments, a therapeutic regimen is effective when the responder score is greater than or equal to 5, 6, 7, 8, 9, or 10. In some embodiments, a therapeutic regimen is effective when the responder score is greater than or equal to 6. In some embodiments, a therapeutic regimen is effective when the responder score is greater than or equal to 7. In some embodiments, a therapeutic regimen is effective when the responder score is greater than or equal to 8. In some embodiments, the therapeutic regimen is not considered effective when the responder score is less than or equal to 8, 7, 6, 5, 4, 3, 2 or 1. In some embodiments, the therapeutic regimen is not considered effective when the responder score is less than or equal to 6.5. In some embodiments, the therapeutic regimen is not considered effective when the responder score is less than or equal to 6. In some embodiments, the therapeutic regimen is not considered effective when the responder score is less than or equal to 5.5.

In some embodiments, the methods, systems, and/or computer readable media disclosed herein further comprise recommending one or more therapeutic regimens based on the responder score. In some embodiments, the methods, systems, and/or computer readable media disclosed herein further comprise administering one or more therapeutic regimens based on the responder score. In some embodiments, the methods, systems, and/or computer readable media disclosed herein further comprise modifying one or more therapeutic regimens based on the responder score. In some embodiments, the methods, systems, and/or computer readable media disclosed herein further comprise terminating one or more therapeutic regimens based on the responder score.

In some embodiments, the therapeutic regimen comprises one or more immune-based anti-cancer therapies. The therapeutic regimen may comprise a T-cell based anti-cancer therapy. The therapeutic regimen may comprise a checkpoint blockade therapy, tumor infiltrating lymphocyte, an anti-cancer vaccine.

In some embodiments, the therapeutic regimen comprises one or more immune-based anti-pathogenic therapies. The therapeutic regimen may comprise one or more immune-based anti-viral therapies. The therapeutic regimen may comprise one or more immune-based anti-bacterial therapies. The therapeutic regimen may comprise one or more immune-based anti-fungal therapies.

Epitopes

The methods, systems, and/or computer readable media disclosed herein comprise determining the immunogenicity of one or more epitope. An epitope may be a fragment of a protein. An epitope may comprise at least 3, 4, 5, 6, 7, 8, 9, 10, 11, 12, 13, 14, or 15 or more amino acids. In some embodiments, an epitope comprises 6 or more amino acids. In some embodiments, an epitope comprises 7 or more amino acids. In some embodiments, an epitope comprises 8 or more amino acids. In some embodiments, an epitope comprises 9 or more amino acids. In some embodiments, an epitope comprises 10 or more amino acids. In some embodiments, an epitope comprises 11 or more amino acids.

The epitopes disclosed herein may be a fragment of a protein expressed in a cell. The cell may be a eukaryotic cell. The cell may be a mammalian cell. Examples of mammals include, but are not limited to, monkeys, cows, sheep, horses, dog, and humans. The cell may be a human cell.

In some embodiments, the epitope is a neoepitope. As used herein, the term “neoepitope” refers an epitope of a neoantigen, such that the neoepitope is a fragment of a neoantigen. As used herein, the term “neoantigen” refers to an antigen that is encoded by tumor-specific mutated genes.

In some embodiments, the epitope is a fragment of a tumor associated antigen. As used herein, the phrase “tumor associated antigen” refers to an antigen that is expressed at a higher level on a cancerous cell as compared to a non-cancerous cell.

In some embodiments, the epitope is a viral epitope. As used herein, the phrase “viral epitope” refers to a fragment of a viral protein.

In some embodiments, the epitope is a bacterial epitope. As used herein, the phrase “bacterial epitope” refers to a fragment of a bacterial protein.

In some embodiments, the epitope is a fungal epitope. As used herein, the phrase “fungal epitope” refers to a fragment of a fungal protein.

In some embodiments, the epitope is a parasitic epitope. As used herein, the phrase “parasitic epitope” refers to a fragment of a parasitic protein.

Indications

The methods, systems, and computer readable media disclosed herein may comprise determining the efficacy of a therapeutic regimen for treating a disease in a subject. The methods, systems, and computer readable media disclosed herein may comprise recommending a therapeutic regimen for treating a disease in a subject. The methods, systems, and computer readable media disclosed herein may comprise modifying a therapeutic regimen for treating a disease in a subject. The methods, systems, and computer readable media disclosed herein may comprise developing an immune-based therapy based on the identification of a potentially immunogenic epitope. The methods, systems, and computer readable media disclosed herein may comprise terminating the development of an immune-based therapy when an epitope is determined to be non-immunogenic.

In some embodiments, the subject described herein suffers from one or more diseases. In some embodiments, the disease is selected from the group consisting of a neoplasia, pathogenic infection, and inflammatory disease.

In some embodiments, the disease is neoplasia. As used herein, the term “neoplasia” refers to a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasia can affect a variety of cell types, tissues, or organs, including but not limited to an organ selected from the group consisting of bladder, colon, bone, brain, breast, cartilage, glia, esophagus, fallopian tube, gallbladder, heart, intestines, kidney, liver, lung, lymph node, nervous tissue, ovaries, pleura, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasias include cancers, such as sarcomas, carcinomas, or plasmacytomas (malignant tumor of the plasma cells). Examples of cancer include, but are not limited to, breast cancer, lung cancer, kidney cancer, colon cancer, renal carcinoma, urothelial carcinoma, Hodgkin's lymphoma, and Merkel cell carcinoma. In some embodiments, the cancer is selected from melanoma, non-small cell lung cancer (NSCLC), cutaneous squamous skin carcinoma, small cell lung cancer (SCLC), hormone-refractory prostate cancer, triple-negative breast cancer, microsatellite instable tumor, renal cell carcinoma, urothelial carcinoma, Hodgkin's lymphoma, and Merkel cell carcinoma.

In some embodiments, the disease is a pathogenic infection. In some embodiments, the pathogenic infection is a viral infection. In some embodiments, the viral infection is selected from an Epstein Barr virus (EBV) infection, cytomegalovirus (CMV) infection, herpes simplex virus (HSV) infection, human herpes virus (HHV) infection, human immunodeficiency virus (HIV) infection, and adenovirus infection. In some embodiments, the EBV infection is EBV reactivation. In some embodiments, the CMV infection is CMV reactivation. In some embodiments, the EBV and/or CMV reactivation occurs in a subject after the subject has experienced an immune suppressive condition. For instance, the EBV and/or CMV reactivation occurs in a subject after the subject has undergone an organ transplantation. Alternatively, or additionally, the EBV and/or CMV reactivation occurs in the subject after the subject has been administered one or more immunosuppressive therapies. In some embodiments, the HSV infection is an HSV1 infection. In some embodiments, the HHV infection is an HHV6 infection. In some embodiments, the pathogenic infection is a bacterial infection. In some embodiments, the bacterial infection is selected from Pseudomonas, Stenotrophomonas, Clostridium, Staphylococcus, and Escherichia. In some embodiments, the Pseudomonas is Pseudomonas aeruginosa. In some embodiments, the Stenotrophomonas is Stenotrophomonas maltophilia. In some embodiments, the Clostridium is Clostridium difficile. In some embodiments, the Staphylococcus is Staphylococcus aureus. In some embodiments, the Escherichia is Escherichia coli. In some embodiments, the bacterial infection is multiresistant Pseudomonas aeruginosa. In some embodiments, the pathogenic infection is a fungal infection. In some embodiments, the fungal infection is selected from Cryptococcus neoformans infection, blastomycosis, Candida auris infection, mucormycosis, aspergillosis, candidiasis, C. gattii infection, ringworm, talaromycosis, and Coccidioidomycosis. In some embodiments, the fungal infection is a Cryptococcus neoformans infection. In some embodiments, the infection is a parasitic infection. In some embodiments, the parasitic infection is selected from toxoplasmosis, trichomoniasis, giardiasis, cryptosporidiosis, and malaria. In some embodiments, the parasitic infection is toxoplasmosis.

Therapeutic Regimens

Further disclosed herein are methods of treating a disease in a subject in need thereof. Generally, the method may comprise administering one or more therapies. The therapy may be administered based on whether the subject is determined to be a responder to the therapy. Alternatively, or additionally, the method may comprise modifying one or more therapies. Modifying the therapeutic regimen may comprise increasing the dose and/or dosing frequency of a therapy. For instance, the therapy may be modified based on whether the subject is determined to be a responder to the therapy or the efficacy of the therapy. The dose or dosing frequency of a therapy may be increased upon determining that the subject is a responder to the therapy, but the current dose or dosing frequency is not effective. Alternatively, the dose or dosing frequency of a therapy may be increased in order to increase the efficacy of the therapy. In some embodiments, modifying the therapy comprises terminating the therapy. In some embodiments, the therapy is selected from an anti-cancer therapy, anti-viral therapy, anti-bacterial therapy, anti-parasitic therapy, and anti-fungal therapy.

In some embodiments, the methods disclosed herein comprise administering one or more anti-cancer therapies. In some embodiments, the methods disclosed herein comprise modifying one or more anti-cancer therapies. Alternatively, or additionally, the methods disclosed herein may comprise terminating one or more anti-cancer therapies. In some embodiments, one or more anti-cancer therapies are selected from an immune checkpoint blockade therapy, vaccine therapy, TCR engineered T cell therapy, adoptive T cell therapy, immune adjuvant therapy, cytokine therapy, interferon therapy, hematopoietic stem cell therapy, gene therapy, CAR T cell therapy, antibody therapy, chemotherapy, and radiation therapy. In some embodiments, the anti-cancer therapy is an immune checkpoint blockade therapy. In some embodiments, the immune checkpoint blockade therapy is selected from an anti-PD1 therapy, anti-PDL1 therapy, and anti-CTLA4 therapy.

In some embodiments, the methods disclosed herein comprise administering one or more anti-viral therapies. In some embodiments, the methods disclosed herein comprise modifying one or more anti-viral therapies. Alternatively, or additionally, the methods disclosed herein may comprise terminating one or more anti-viral therapies. In some embodiments, the one or more anti-viral therapies is selected from 5-substituted 2′-deoxyuridine analogues, nucleoside analogues, pyrophosphate analogues, NRTIs, NNRTIs, protease inhibitors, integrase inhibitors, entry inhibitors, acyclic guanosine analogues, acyclic nucleoside phosphonate analogues, HCV NSSA and NSSB inhibitors, influenza virus inhibitors, interferons, immunostimulators, oligonucleotides, antimitotic inhibitors, and adoptive T cell transfers specific for the infecting agent.

In some embodiments, the methods disclosed herein comprise administering one or more anti-bacterial therapies. In some embodiments, the methods disclosed herein comprise modifying one or more anti-bacterial therapies. Alternatively, or additionally, the methods disclosed herein may comprise terminating one or more anti-bacterial therapies. In some embodiments, the one or more anti-bacterial therapies is selected from beta-lactams (penicillins, cephalosporins, carbapenems), monobactams, glycopeptides, cyclic lipopeptides, streptogramins, fluoroquinolons, aminoglycosides, macrolides, tetracyclines, glycylcyclines, lincosamides, folate antagonists, oxazolidinones, nitroimidazoles, nitrofurans, rifamycins, and polymyxins.

In some embodiments, the methods disclosed herein comprise administering one or more anti-fungal therapies. In some embodiments, the methods disclosed herein comprise modifying one or more anti-fungal therapies. Alternatively, or additionally, the methods disclosed herein may comprise terminating one or more anti-fungal therapies. In some embodiments, the one or more anti-fungal therapies is selected from azoles, polyenes, allylamines, echinocandins, pyrimidine analogues, mitotic inhibitors and vaccines.

In some embodiments, the methods disclosed herein comprise administering one or more anti-parasitic therapies. In some embodiments, the methods disclosed herein comprise modifying one or more anti-parasitic therapies. Alternatively, or additionally, the methods disclosed herein may comprise terminating one or more anti-parasitic therapies. In some embodiments, the one or more anti-parasitic therapies is selected from nitroimidazoles, pyrimethamine, cycloguanil, sulphones or sulphonamides, atovaquone, fosmidomycin, difluoromethylornithine, triazoles, bisphosphonates, levamisole, albendazole, ivermectin.

Compositions

Further disclosed herein are compositions comprising one or more non-immunogenic epitopes. Also disclosed herein are compositions comprising one or more polynucleotides that encode one or more non-immunogenic epitopes. Further disclosed herein are agents that specifically bind to one or more non-immunogenic epitopes.

Further disclosed herein are compositions comprising a non-immunogenic epitope listed in any of Tables 2-4. In some embodiments, the composition comprises a plurality of non-immunogenic epitopes listed in any of Tables 2-4. In some embodiments, the composition comprises at least 2, 3, 4, 5, 6, 7, 8, 9, 10, 11, 12, 13, 14, 15, 16, 17, 18, 19, or 20 or more non-immunogenic epitopes listed in any of Tables 2-4. In some embodiments, the composition comprises a non-immunogenic epitope listed in Table 2. In some embodiments, the composition comprises a non-immunogenic epitope listed in Table 3. In some embodiments, the composition comprises a non-immunogenic epitope listed in Table 4.

Further disclosed herein are compositions comprising polynucleotides encoding a non-immunogenic epitope listed in any of Tables 2-4. In some embodiments, the composition comprises (a) a polynucleotide encoding an epitope listed in any of Tables 2-4; and (b) a bacterial plasmid, wherein the polynucleotide is inserted into the bacterial plasmid. In some embodiments, the polynucleotide encodes an epitope listed in Table 2. In some embodiments, the polynucleotide encodes an epitope listed in Table 3. In some embodiments, the polynucleotide encodes an epitope listed in Table 4.

In some embodiments, the polynucleotide comprises deoxyribonucleic acid (DNA). In some embodiments, the bacterial plasmid further comprises a eukaryotic promoter.

Further disclosed herein is a composition comprising (a) a polynucleotide encoding an epitope listed in any of Tables 2-4; and (b) a polymerase. In some embodiments, the polynucleotide comprises deoxyribonucleic acid (DNA). In some embodiments, the polymerase is a RNA polymerase. In some embodiments, the polymerase is a bacteriophage polymerase. In some embodiments, the polymerase is a bacteriophage RNA polymerase. In some embodiments, the polynucleotide encodes an epitope listed in Table 2. In some embodiments, the polynucleotide encodes an epitope listed in Table 3. In some embodiments, the polynucleotide encodes an epitope listed in Table 4.

Further disclosed herein is a composition comprising a plurality of polynucleotides encoding a plurality of epitopes listed in any of Tables 2-4. In some embodiments, the plurality of polynucleotides comprises at least 2, 3, 4, 5, 6, 7, 8, 9, 10, 11, 12, 13, 14, 15, 16, 17, 18, 19, or 20 or more polynucleotides that encode at least 2, 3, 4, 5, 6, 7, 8, 9, 10, 11, 12, 13, 14, 15, 16, 17, 18, 19, or 20 or more different epitopes listed in Tables 2-4. In some embodiments, the polynucleotide encodes an epitope listed in Table 2. In some embodiments, the polynucleotide encodes an epitope listed in Table 3. In some embodiments, the polynucleotide encodes an epitope listed in Table 4.

Further disclosed herein is a composition comprising (a) an agent that specifically binds to one or more non-immunogenic epitopes listed in any of Tables 2-4; and (b) a solid support. In some embodiments, the agent is a human leukocyte antigen (HLA). In some embodiments, the solid support is selected from a bead, array, slide, and multiwell plate. In some embodiments, the agent specifically binds to a non-immunogenic epitope listed in Table 2. In some embodiments, the agent specifically binds to a non-immunogenic epitope listed in Table 3. In some embodiments, the agent specifically binds to a non-immunogenic epitope listed in Table 4. In some embodiments, the agent is a human leukocyte antigen (HLA).

Further disclosed herein is a composition comprising (a) an agent that specifically binds to one or more non-immunogenic epitopes listed in any of Tables 2-4; and (b) a reporter molecule. In some embodiments, the agent specifically binds to a non-immunogenic epitope listed in Table 2. In some embodiments, the agent specifically binds to a non-immunogenic epitope listed in Table 3. In some embodiments, the agent specifically binds to a non-immunogenic epitope listed in Table 4. In some embodiments, the agent is a human leukocyte antigen (HLA).

In some embodiments, the reporter molecule is selected from a fluorophore, chemiluminescent molecule, and an antibiotic resistance protein.

### Definitions

Unless defined otherwise, all technical and scientific terms used herein have the meaning commonly understood by a person skilled in the art to which this disclosure belongs. The following references provide one of skill with a general definition of many of the terms used in the present technology: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

As used herein, the singular forms “a”, “an” and “the” are intended to include the plural forms as well, unless the context clearly indicates otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “administration” of an agent to a subject includes any route of introducing or delivering the agent to a subject to perform its intended function. Administration can be carried out by any suitable route, including, but not limited to, intravenously, intramuscularly, intraperitoneally, subcutaneously, and other suitable routes as described herein. Administration includes self-administration and the administration by another.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refer to agents that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. In some embodiments, amino acids forming a polypeptide are in the D form. In some embodiments, the amino acids forming a polypeptide are in the L form. In some embodiments, a first plurality of amino acids forming a polypeptide is in the D form and a second plurality is in the L form.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter code.

As used herein, the terms “percentile rank” or “% rank” refer to the rank of the predicted affinity of a peptide (e.g., an epitope, or HLA-LM) to a MHC molecule (e.g., an HLA molecule or HLA allele) compared to a plurality of random natural peptides to the MHC molecule (e.g., an HLA molecule or HLA allele). This measure is not affected by inherent bias of certain molecules towards higher or lower mean predicted affinities.

The terms “polypeptide,” “peptide,” and “protein” are used interchangeably herein to refer to a polymer of amino acid residues. The terms apply to naturally occurring amino acid polymers as well as amino acid polymers in which one or more amino acid residues is a non-naturally occurring amino acid, e.g., an amino acid analog. The terms encompass amino acid chains of any length, including full length proteins, wherein the amino acid residues are linked by covalent peptide bonds.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a composition known to exhibit the desired therapeutic effect) and a negative control (a subject or a sample that does not receive the therapy or receives a placebo) are typically employed.

As used herein, the term “effective amount” or “therapeutically effective amount” refers to a quantity of an agent sufficient to achieve a desired therapeutic effect. In the context of therapeutic applications, the amount of a therapeutic peptide administered to the subject can depend on the type and severity of the infection and on the characteristics of the individual, such as general health, age, sex, body weight and tolerance to drugs. It can also depend on the degree, severity and type of disease. The skilled artisan will be able to determine appropriate dosages depending on these and other factors.

As used herein, “epitopes” refer to a class of major histocompatibility complex (MHC) bounded peptides that are recognized by the immune system as targets for T cells and can elicit an immune response in a subject. “Neoepitopes” refer to epitopes that arise from tumor-specific mutations that may elicit an immune response to cancer. Epitopes usually consist of chemically active surface groupings of molecules such as amino acids or sugar side chains and usually have specific three dimensional structural characteristics, as well as specific charge characteristics.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell. The expression level of a gene can be determined by measuring the amount of mRNA or protein in a cell or tissue sample. In one aspect, the expression level of a gene from one sample can be directly compared to the expression level of that gene from a control or reference sample. In another aspect, the expression level of a gene from one sample can be directly compared to the expression level of that gene from the same sample following administration of the compositions disclosed herein. The term “expression” also refers to one or more of the following events: (1) production of an RNA template from a DNA sequence (e.g., by transcription) within a cell; (2) processing of an RNA transcript (e.g., by splicing, editing, 5′ cap formation, and/or 3′ end formation) within a cell; (3) translation of an RNA sequence into a polypeptide or protein within a cell; (4) post-translational modification of a polypeptide or protein within a cell; (5) presentation of a polypeptide or protein on the cell surface; and (6) secretion or presentation or release of a polypeptide or protein from a cell.

As used herein, the term “ligand” refers to a molecule that binds to a second molecule. The ligand may have a binding affinity for the second molecule of less than or equal to 10000; 9500; 9000; 8500; 8000; 7500; 7000; 6500; 6000; 5500; 5000; 4500; 4000; 3500; 3000; 2500; 2000; 1500; 1000; 900; 800; 700; 600; or 500 nM. The ligand may have a binding affinity for the second molecule of less than or equal to 8000 nM. The ligand may have a binding affinity for the second molecule of less than or equal to 6000 nM. The ligand may have a binding affinity for the second molecule of less than or equal to 5000 nM. The ligand may have a binding affinity for the second molecule of less than or equal to 4000 nM. The ligand may have a binding affinity for the second molecule of less than or equal to 2000 nM. The ligand may have a binding affinity for the second molecule of less than or equal to 1000 nM. The ligand may have a binding affinity for the second molecule of less than or equal to 500 nM. In some embodiments, the ligand is an epitope disclosed herein and the second molecule is a MHC protein, such as an HLA.

As used herein, “major histocompatibility complex (MHC)” refers to a group of genes that code for proteins found on the surfaces of cells that help the immune system recognize foreign substances. MHC proteins are found in all higher vertebrates. In human beings the complex is also called the human leukocyte antigen (HLA) system. HLAs corresponding to MHC class I (A, B, and C) which all are the HLA Class1 group present peptides from inside the cell. In general, these particular peptides are small polymers, about 9 amino acids in length. Foreign antigens presented by MHC class I attract killer T-cells (also called CD8 positive- or cytotoxic T-cells) that destroy cells. HLAs corresponding to MHC class II (DP, DM, DO, DQ, and DR) present antigens from outside of the cell to T-lymphocytes. These particular antigens stimulate the multiplication of T-helper cells (also called CD4 positive T cells), which in turn stimulate antibody-producing B-cells to produce antibodies to that specific antigen. Self-antigens are suppressed by regulatory T cells.

As used herein, the term “modulate” refers positively or negatively alter. Exemplary modulations include an about 1%, about 2%, about 5%, about 10%, about 25%, about 50%, about 75%, or about 100% change.

As used herein, the term “increase” refers to alter positively by at least about 5%, including, but not limited to, alter positively by about 5%, by about 10%, by about 25%, by about 30%, by about 50%, by about 75%, or by about 100%.

As used herein, the term “reduce” refers to alter negatively by at least about 5% including, but not limited to, alter negatively by about 5%, by about 10%, by about 25%, by about 30%, by about 50%, by about 75%, or by about 100%.

### EXAMPLES

The practice of the present technology employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the present technology, and, as such, can be considered in making and practicing the present technology. Particularly useful techniques for particular embodiments will be discussed in the sections that follow.

The following examples are put forth so as to provide those of ordinary skill in the art with a complete disclosure and description of how to make and use the compositions, and assay, screening, and therapeutic methods of the present technology, and are not intended to limit the scope of what the inventors regard as the present technology.

### Example 1: Identification of Non-Immunogenic Neoepitopes Predicts Response to Immune Checkpoint Blockade Therapy

T cell responses against neoepitopes represent a critical mediator of effective anti-cancer immunity^{1,2}. However, only a small fraction of neoepitopes elicits immune responses in vitro and in vivo³, making development of tumor-specific therapies more difficult. In this example, a model is developed to investigate whether T cell reactivity is limited mostly by pre-existing T cell tolerance to non-mutated, normally presented human leukocyte antigen (HLA) ligands. Briefly, a model was developed to predict tolerance against neoepitopes based on their physicochemical similarity to non-mutated HLA class I ligands identified by mass spectrometry (MS). This model prospectively predicts non-immunogenic neoepitopes with high positive predictive value (97%) and postulates a novel mechanism, which is termed “allelic cross-tolerance”. Without being bound by theory, this mechanism is based on the assumption that high similarity between a neoepitope and a non-mutated self-peptide at their T cell receptor recognition areas can be sufficient to confer tolerance to the neoepitope, which is independent of its presenting HLA allele, but dependent on the HLA allele repertoire of the patient. Furthermore, utilizing these novel insights and acknowledging non-immunogenicity of a large fraction of neoepitopes, this example demonstrates an exemplary use of a “RESPONDER” score which predicts patients' responses to checkpoint blockade therapy with unprecedented precision. Altogether, this model predicted non-immunogenicity of neoepitopes as well as response to immune checkpoint blockade therapy and supported a novel explanation for tolerance to certain neoepitopes. The use of this model to characterize the immunogenicity of a neoepitope may facilitate the design of neoepitope-based therapies and spare many potentially unresponsive patients from toxicities and costs of immune checkpoint blockade (ICB) therapy.

Immune checkpoint blockade (ICB) is emerging as an effective therapy for many cancers. In addition, neoantigen-based vaccination strategies have been shown to be safe and active in clinical trials, but typically a substantial fraction of the targeted neoepitopes are not capable of eliciting immune responses⁴⁻⁷. While a wide range of immunosuppressive mechanisms⁸⁻¹¹ may influence a patient's T cell responses in vivo, T cells from healthy individuals can also show considerable variance in reactivity when challenged with neoepitopes in vitro¹². A reliable explanation for this phenomenon is lacking. Understanding the underlying mechanisms of T cell reactivity would facilitate the selection of suitable targets for neoepitope-based immunotherapies but would also significantly improve the most commonly used biomarker for response to ICB, tumor mutational load¹³, as non-immunogenic mutations could be sorted out a priori. Without being bound by theory, one explanation for non-reactivity of neoepitopes might be a pre-existing tolerance to these neoepitopes. During negative thymic selection, T cells recognizing self-peptides undergo apoptosis; thus, HLA ligands commonly presented on the mature cell surface are non-reactive¹⁴. Therefore, the normal immunopeptidome might serve as a surrogate for non-immunogenic ligands. Hence, if a cancer mutation-derived neoepitope shares high similarity in physico-chemical and binding characteristics with an unmutated HLA ligand, the neoepitope would very likely be non-immunogenic as well.

Recently, strategies to identify HLA ligands have improved dramatically. Advancements in biochemical isolation and subsequent analysis via mass spectrometryl^{5,16} as well as peptide sequence identification through annotation algorithms¹⁷⁻¹⁹ now allow reliable detection of thousands of unique HLA ligands with high-confidence^{20,21}. Additionally, assignment predictions for peptides to their presenting HLA complex enable HLA allele-specific analysis of the immunopeptidome^{22,23}.

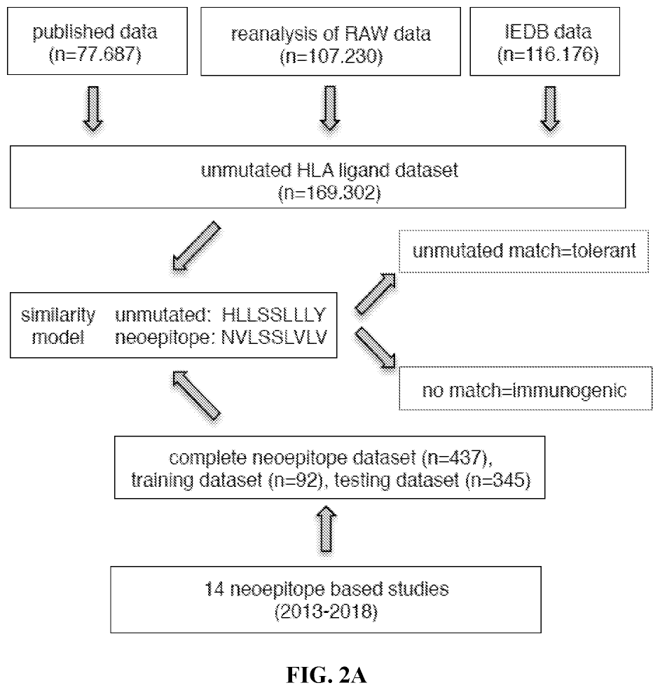

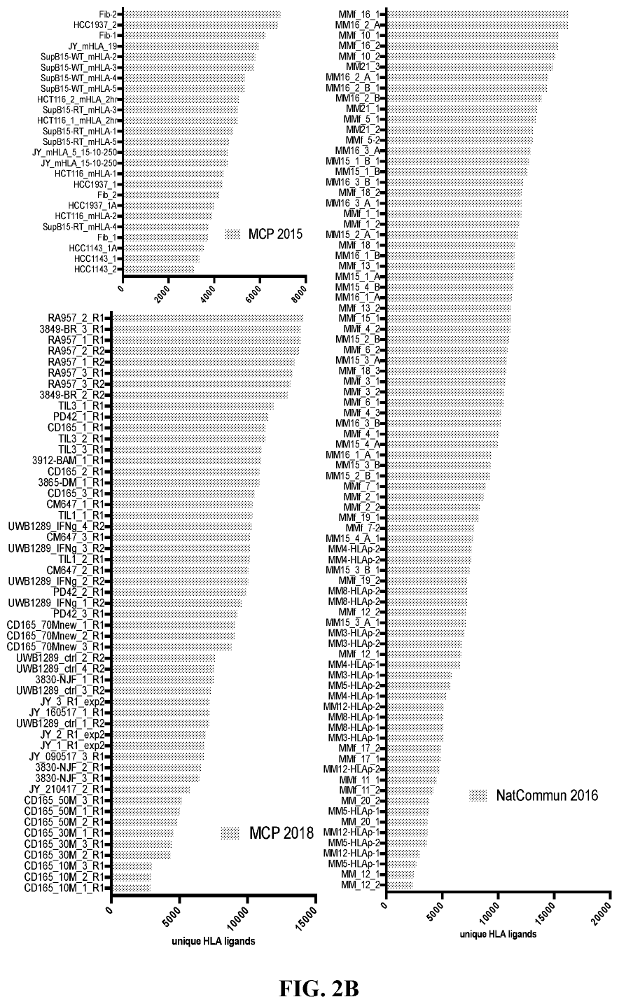

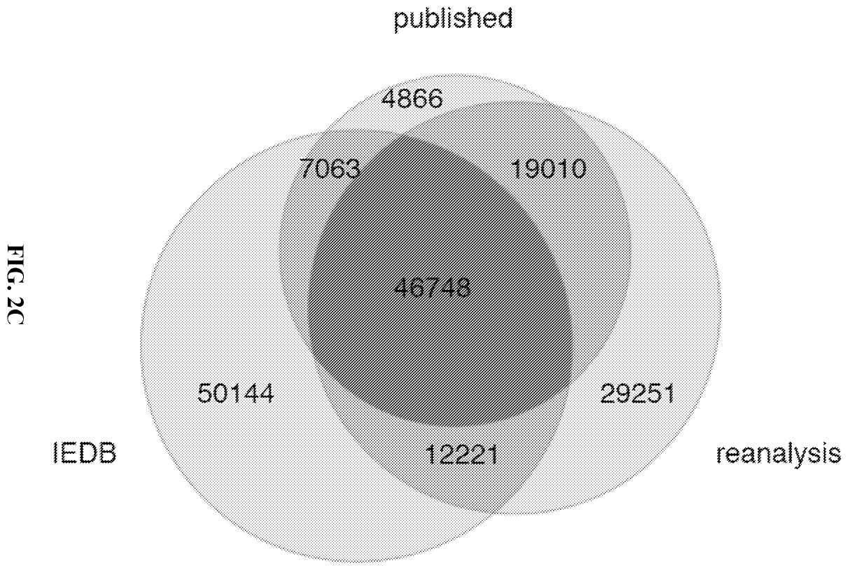

To provide an extensive dataset of non-mutated self-peptides for the present studies, three sources were utilized (FIG. 2A): 1) MS-identified HLA class I 9mer peptides from the IEDB database²⁴ (data cutoff Sep. 20, 2018) resulting in 116,176 unique peptides. 2) datasets from previously published studies that yielded large numbers of HLA ligands not included in IEDB ^{15,20,21} leading to 77,687 unique peptides. 3) re-analysis of the mass spectra from aforementioned studies (161 RAW files retrieved from PRIDE archive²⁶) using the highly sensitive byonic software²⁵ and assigning resulting peptides to the HLA alleles provided by these studies via netMHCpan 4.0²². On average, the re-analysis yielded 8,400 unique 8-12mer HLA ligands per run and up to 16,000 in a single analysis (FIG. 2B). The total number of unique 9mer peptides was 107,230. After combining these three sources, an extensive dataset of 169,302 unique 9mer HLA ligands was created. Intriguingly, re-analysis identified over 29,000 previously undescribed peptides, expanding the MS-identified 9mer data of the IEDB database by 25% (FIG. 2C). In parallel, neoepitope-based studies^{6,7,12,21,27-36} were exploited for point-mutated 9mer HLA ligands for which T cell reactivity data as well as HLA typing of patients were available and collected 437 hits (FIG. 2A). T cell reactivity was determined in these studies either by multimer or ELISpot assays. Of these 437 peptides, 84 were reactive and 353 were not.

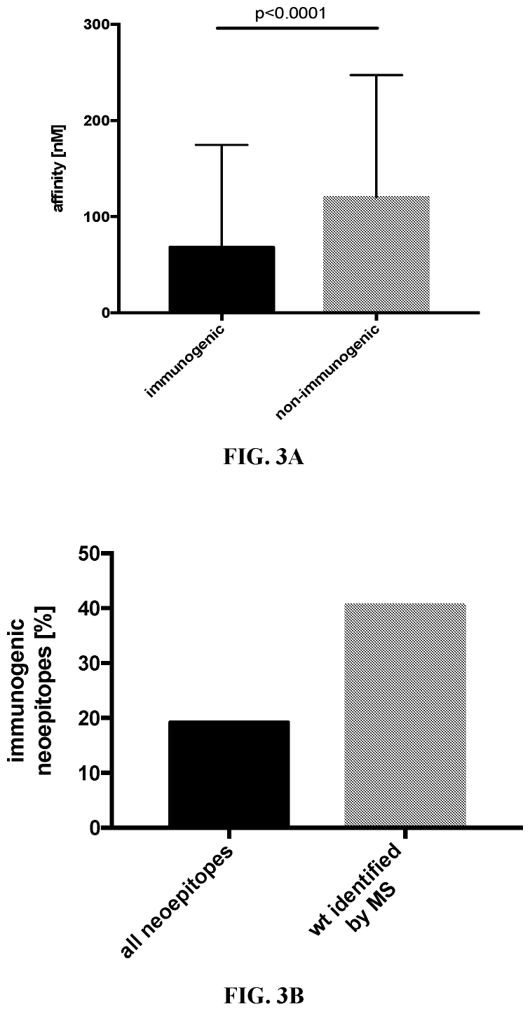

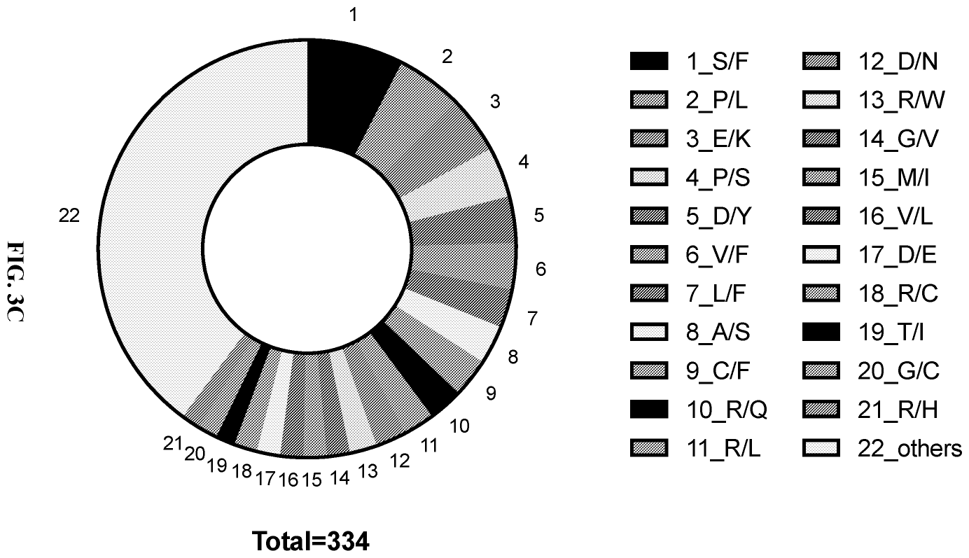

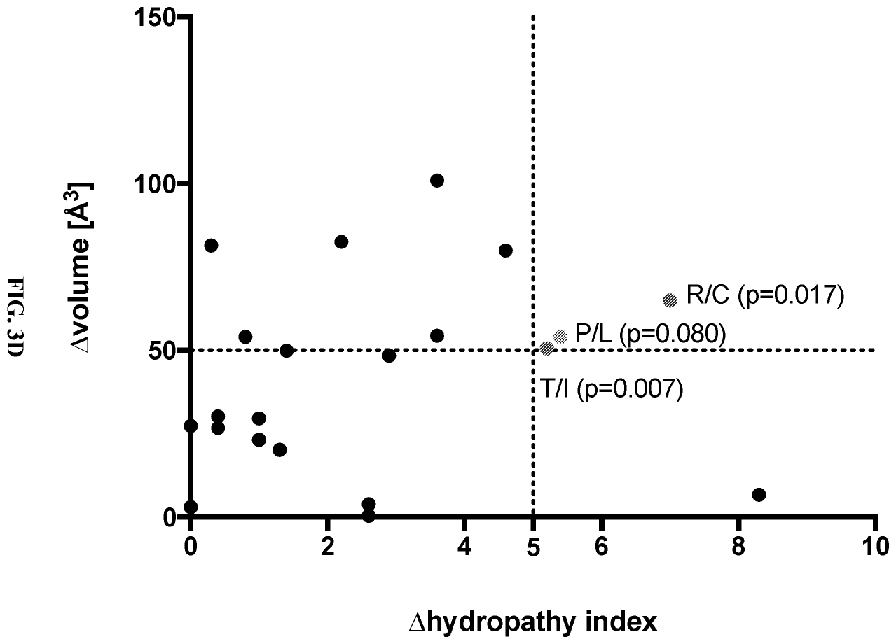

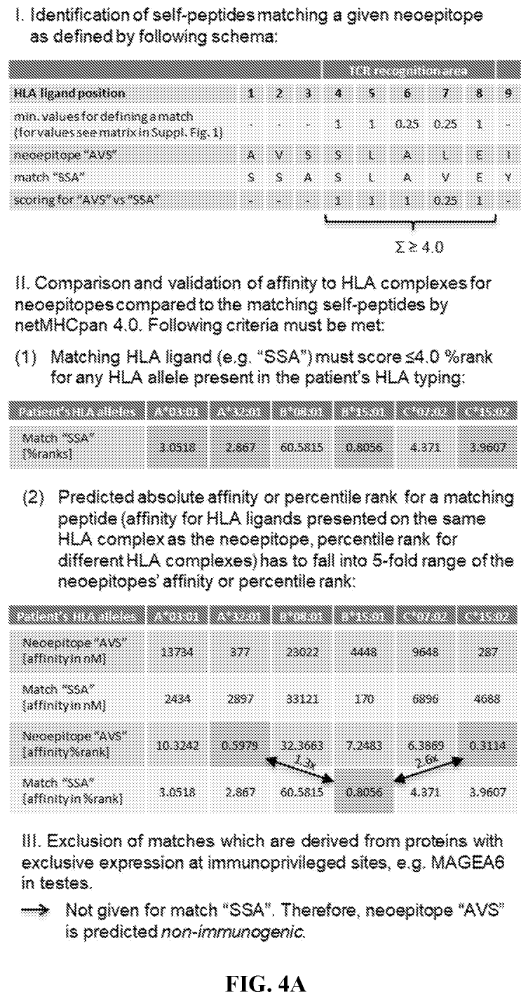

The data set was confirmed for the known positive correlation of peptide immunogenicity and peptide-HLA complex affinity³⁷ (FIG. 3A). Next, it was determined whether the wild-type counterpart peptides of the collected neoepitopes could be identified in the MS dataset since most studies rely only on prediction of neoepitopes based on genomic data, but do not provide evidence as to whether these peptides are displayed at the cell surface. Interestingly, for only 42 out of 437 neoepitopes (9.6%), presentation of the wild-type peptide was confirmed by the MS dataset. The fraction of immunogenic peptides within that subgroup was more than 2-fold higher than in the set of all neoepitopes (40.5% vs. 19.3%, respectively) (FIG. 3B). These data suggested that some of the postulated neoepitopes might not be recognized nor immunogenic due to a lack of processing and presentation. Furthermore, to determine if the type of point mutation influences directly the immunogenicity of neoepitopes, point mutations occurring at positions 2 and 9 of the 9mer peptides were excluded, since these positions represent anchor residues and therefore their amino acid side chains are typically not involved in TCR interactions. Only point-mutations that occurred at least five times were included in this analysis. 21 different point mutations were eligible for investigation, representing 60% of all occurring alterations (FIG. 3C). Two kinds of point mutations showed significant enrichment for immunogenicity in this analysis: R to C (p=0.017) and T to I (p=0.007). Both amino acid changes led to substantial increases in hydrophobicity, another well-known characteristic of immunogenic epitopes³⁸. Additionally, if the change in amino acid size was also considered, a clear separation for these two types of point mutations was seen, as compared to the remaining alterations (FIG. 3D). Interestingly, the only mutation with similar characteristics (P to L) did not show significant enrichment in the analysis, but did show a trend (p=0.08) for immunogenicity. Thus, point mutations resulting in combined major changes in hydrophobicity (Δhydrophobicity≥5.0) and size (Δvolume≥50 Å³) increased the chance for immunogenicity, if these changes did not occur at anchor positions 2 or 9.

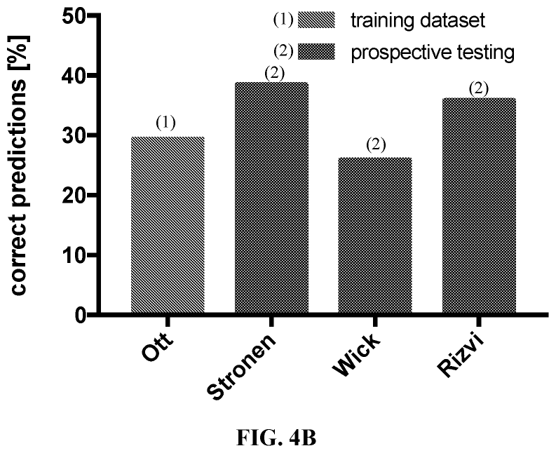

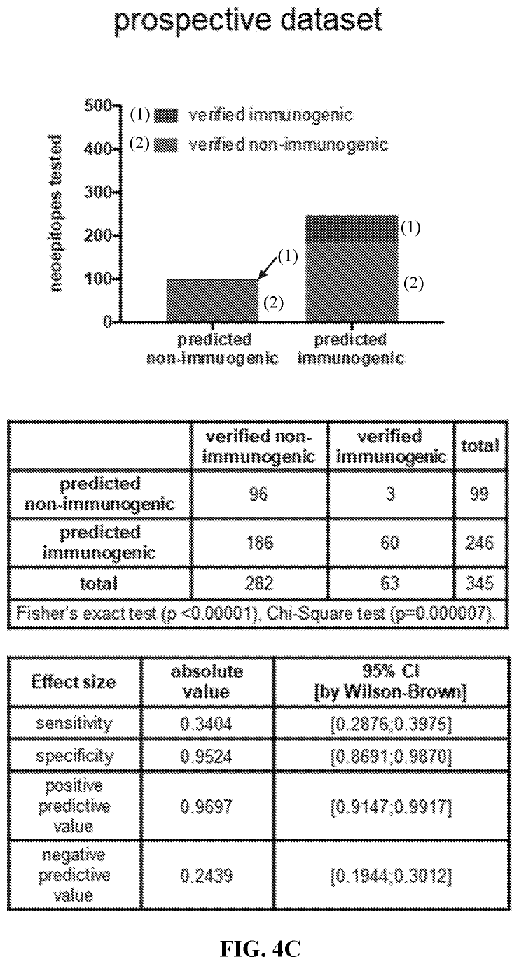

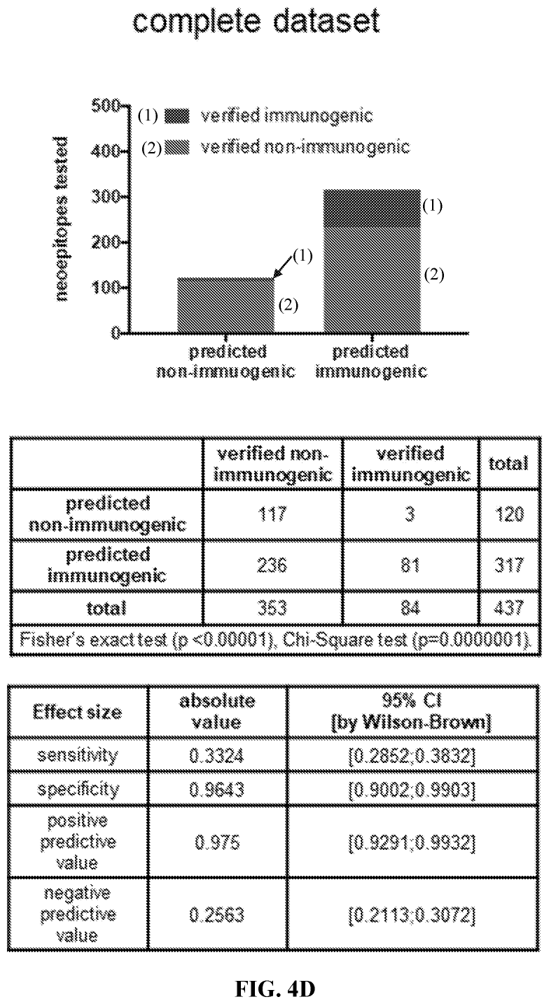

Then, to investigate whether T cell reactivity is limited mostly by pre-existing T cell tolerance to non-mutated, normally presented human leukocyte antigen (HLA) ligands, a prediction model for non-immunogenicity of neoepitopes based on their biochemical similarity and comparable affinity to unmutated normal HLA ligands was designed (FIG. 4A). Three studies including 92 neoepitopes (21 immunogenic, 71 non-immunogenic; immunogenicity was determined by ELISpot and multimer staining assays in published studies from which the neoepitopes were retrieved from) were selected as a training set^{6,21,35} to define the rules for the prediction model that lead to optimal specificity and positive predictive value: First, neoepitopes were compared to the dataset of 169,000 unmutated HLA ligands at positions 4 to 8 since these residues most often form the main chemical interaction with the TCR residues: mutated peptides with amino acids identical to amino acids of normal peptides at positions 4,5 and 8, were identified, since side chains of these three amino acids most commonly interact with the TCR⁴¹. For positions 6 and 7, amino acids of the neoepitope had to be at least physico-chemically similar compared to the non-mutated HLA ligands^{40,42} and similarity was weighted in a scoring matrix (FIG. 4A top and FIG. 6; see detailed description in Methods, below).

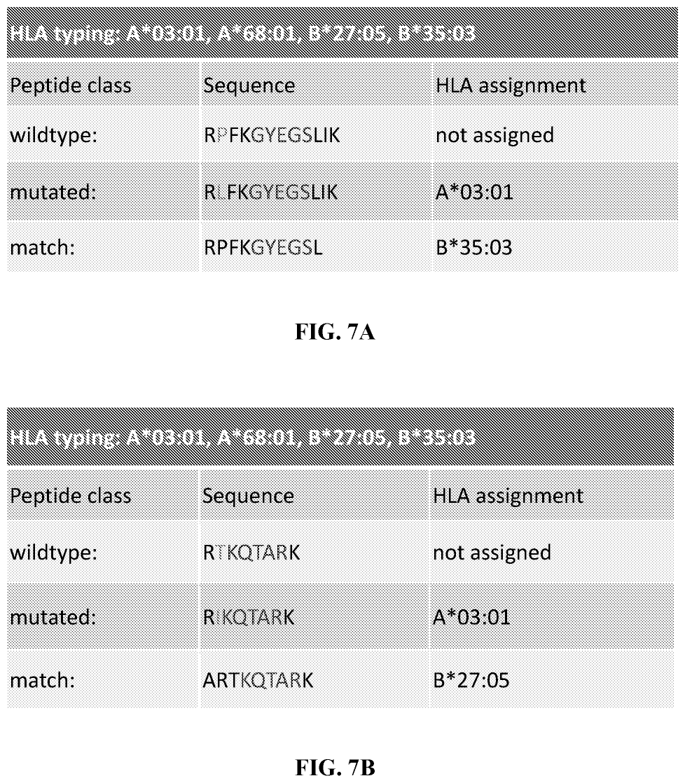

Second, if a matching non-mutated normal HLA ligand was found, its absolute affinity to an HLA complex (in nM) as well as its normalized affinity defined by its percentile rank (now referred to as % rank) for each HLA allele displayed by the patient was calculated by netMHCpan 4.0. Absolute affinity and % rank of the unmutated match had to fall into a 5-fold range compared to the neoepitope's affinity or % rank to still be considered a match (FIG. 4A middle). If the neoepitope and unmutated HLA ligand match were compared for the same HLA allele, absolute affinity was used as parameter. In cases where the match could only be presented on a different HLA complex expressed by the patient, % ranks were used as normalized values to allow an interallelic comparison. The rationale for accepting peptide hits presented on a different HLA allele compared to the neoepitope, and thus ignoring the hallmark of HLA restriction, was provided from the initial re-analysis of MS-identified HLA ligands. Here, this method identified two instances of non-immunogenic neoepitopes (which were verified by MS in the initial study²¹), in which the mutations (both on position 2) enabled presentation of the neoepitope on an HLA-A*03 complex in the patient, in contrast to the cognate wildtype peptide counterpart, which could not be presented by any of the patient's HLA alleles. Surprisingly, length variants of the wildtype peptides were found by MS analyses in the patient's HLA ligandome from the same study, but were presented on different HLA complexes compared to the neoepitope (FIGS. 7A-7B). In these two examples the TCR recognition sites were unchanged and this similarity to the normal peptides might have been the cause of tolerance to these neoepitopes.

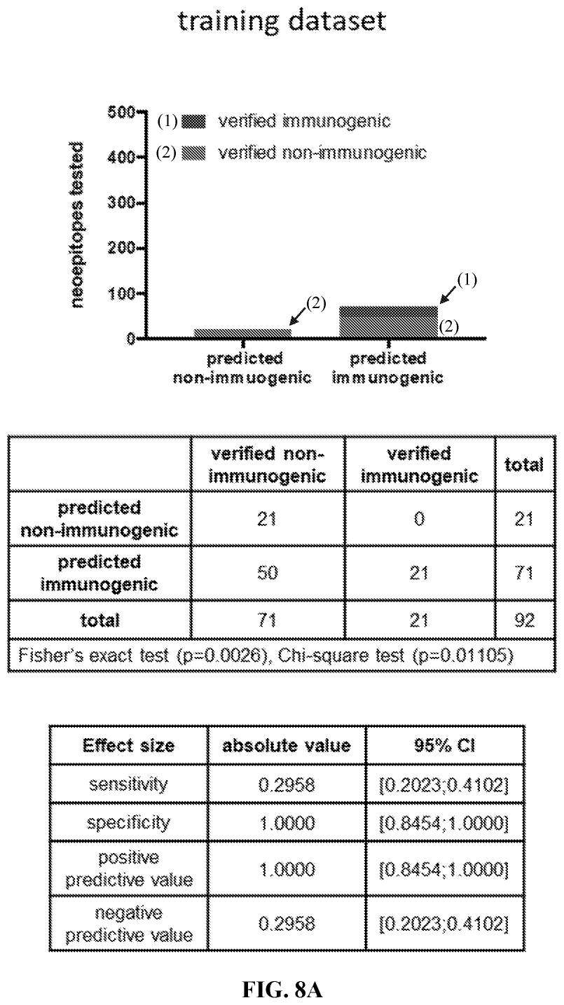

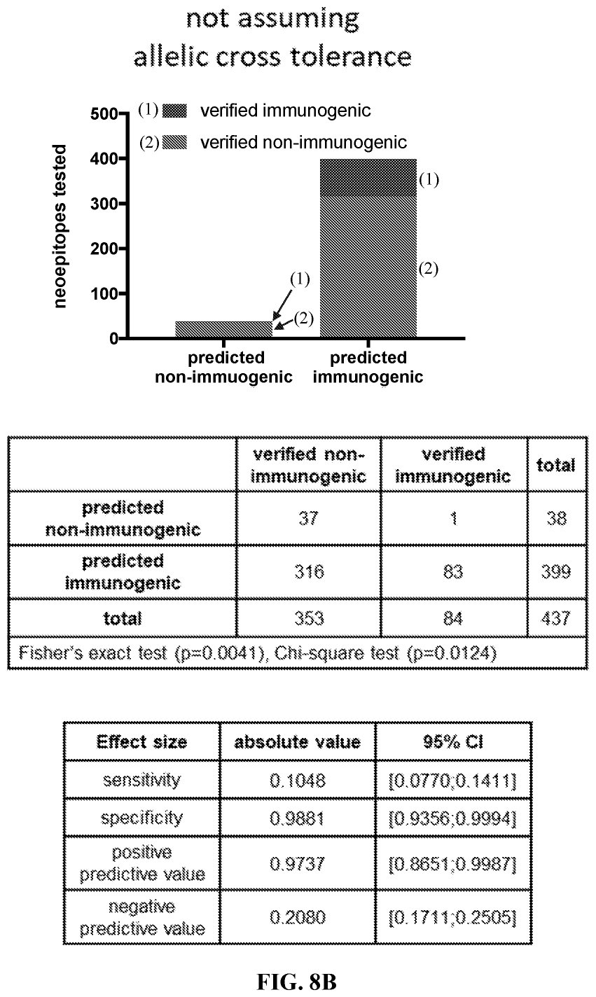

To exclude confounding immunogenic self-peptides from the matches, a third step investigated expression patterns of genes from which the potential peptide matches were derived. If gene expression was restricted to immune-privileged sites, which was observed for 5 peptides (e.g. like MAGEA6 in testis), the match was discarded due to the possible immunogenicity of the unmutated HLA ligand (FIG. 4A bottom and Table 1). Altogether, we then used the training dataset to optimize the prediction model for highest specificity and positive predictive value (FIG. 8A).

TABLE 1

| Peptide | UniProt | Gene | Expression |
|---|---|---|---|
| sequence | identifier | name | pattern |
| KIWEELSML | P43356 | MAGEA2 | testis specific |
| (SEQ ID NO: 1) | | | |
| EVDPIGHVY | P43360 | MAGEA6 | testis specific |
| (SEQ ID NO: 2) | | | |
| SAAAVFSHF | Q4ZJI4 | SLC9B1 | testis specific |
| (SEQ ID NO: 3) | | | |
| KVVAVNDPF | O14556 | GAPDHS | testis specific |
| (SEQ ID NO: 4) | | | |
| TLGTVILLV | Q9UHM6 | OPN4 | eye and CNS |
| (SEQ ID NO: 5) | | | specific |

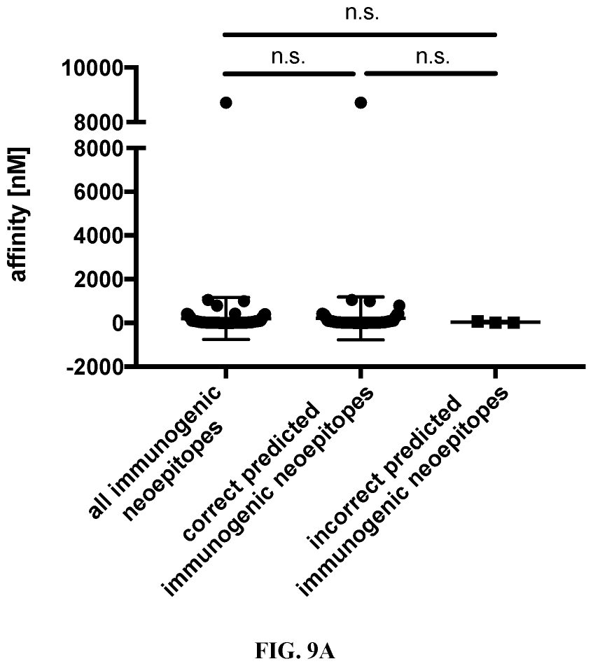

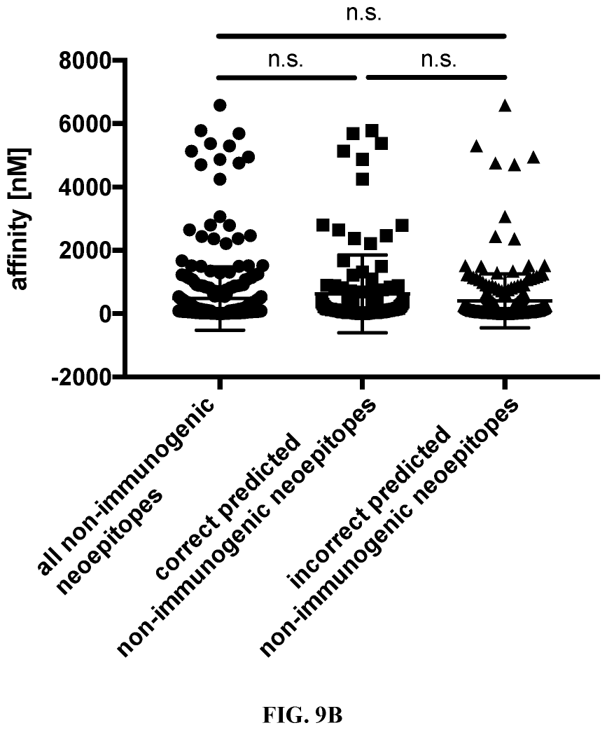

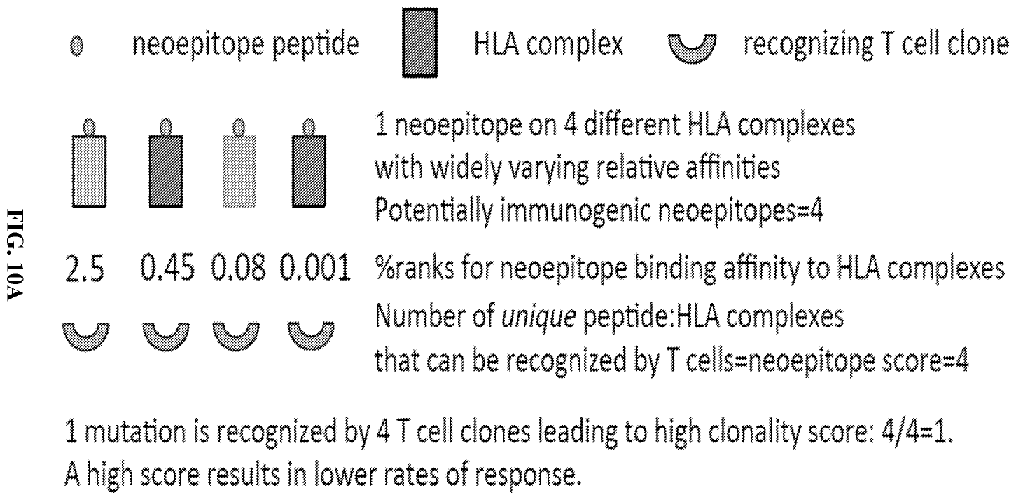

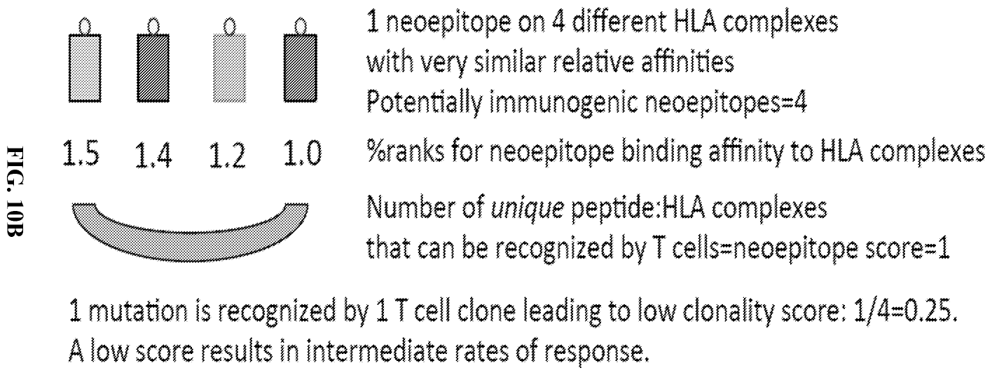

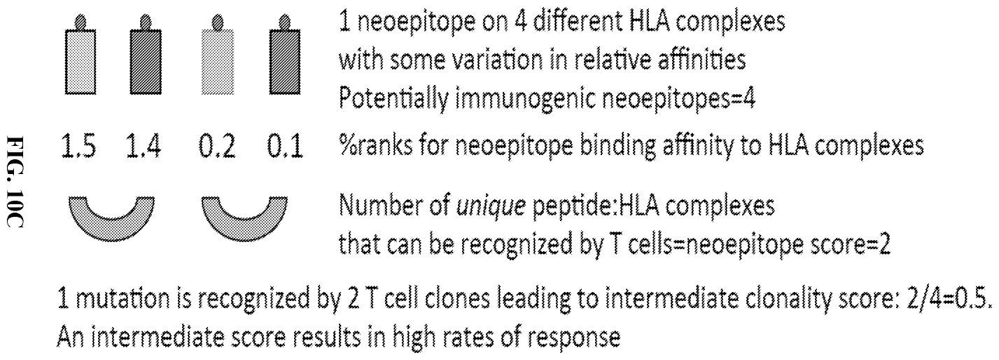

Subsequently, the prediction model was applied to 11 different studies that identified neoepitopes and determined their immunogenicity to prospectively test our performance in prediction of tolerance to neoepitopes. Matches for the non-immunogenic neoepitopes in the examined studies were found to range from 26 to 39% of all neoepitopes tested, offering a potential explanation for lack of T cell reactivity against them, and confirming the sensitivity of our model of 29% observed in our training set (FIG. 4B). During prospective testing for 63 immunogenic neoepitopes, only 3 peptides were predicted to be non-immunogenic (false positive rate of 4.8%). Overall, the model showed excellent specificity (95.2% for prospective testing, 96.4% for the complete dataset) and positive predictive value (97.0% for prospective testing, 97.5% for the complete dataset) for the prediction of non-immunogenicity of point-mutated 9mer neoepitopes in tests of 437 neoepitopes from 14 different studies, thereby demonstrating a highly significant capacity of the model algorithm to predict non-immunogenic neoepitopes (Fisher's exact test, p<0.00001, Chi-Square test, p=1.0×10⁻⁷, FIG. 4C). To exclude affinity of neoepitopes to HLA complexes as a confounding factor in our model that might predetermine a correct or incorrect prediction, peptide affinities among the correctly and incorrectly predicted subgroups were analyzed. No significant differences in affinities were found either for immunogenic nor non-immunogenic HLA ligands (FIGS. 9A-9B).

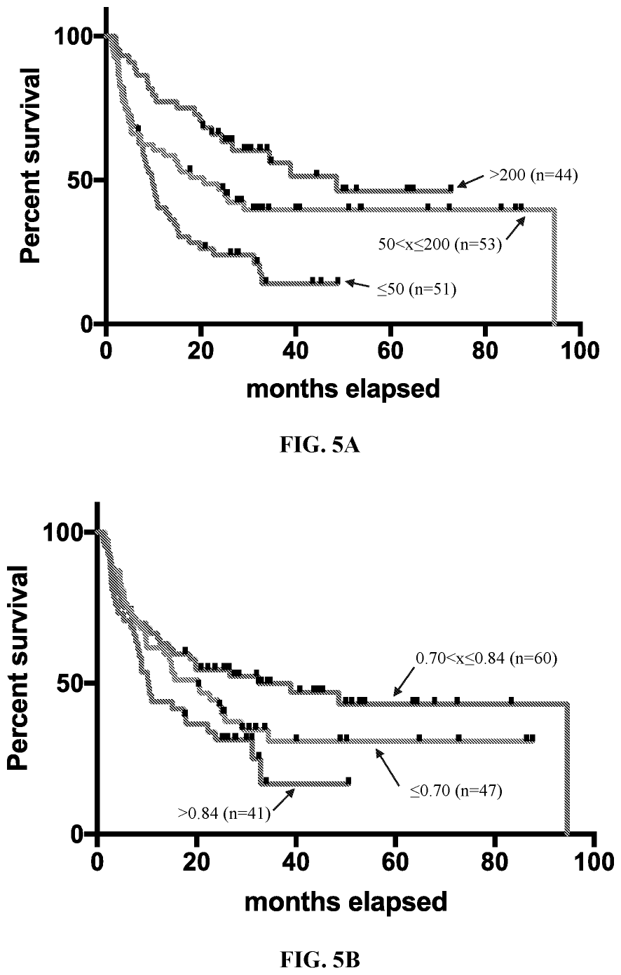

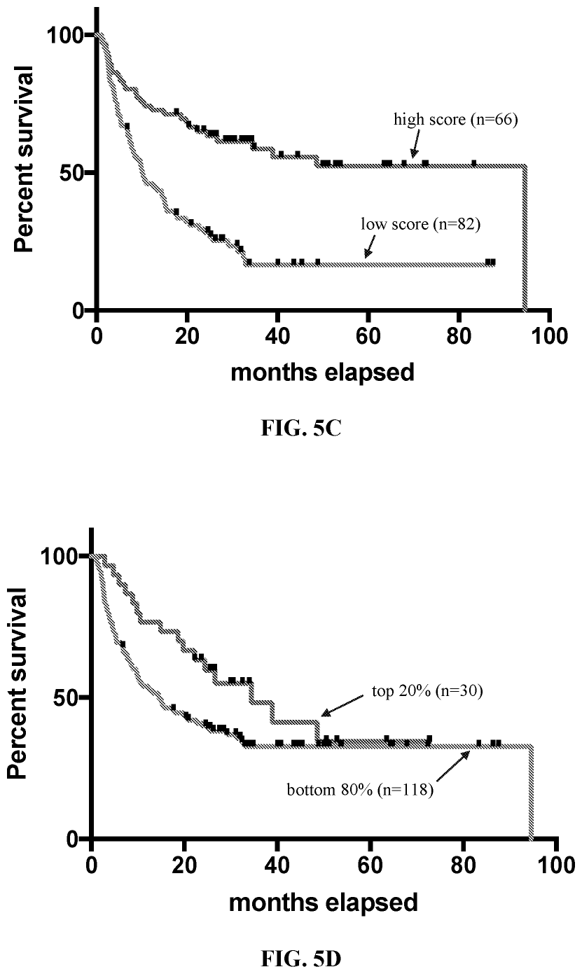

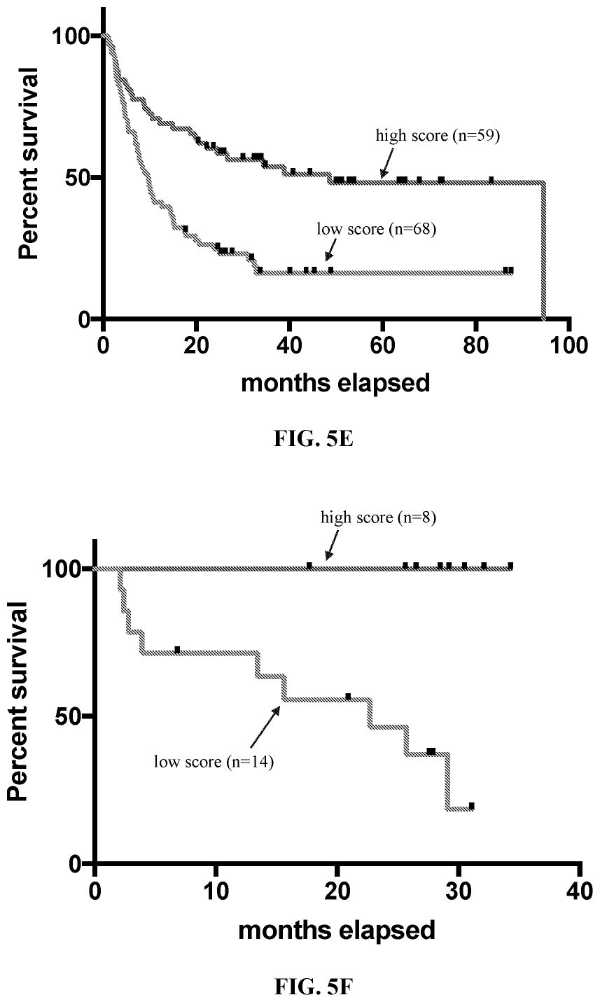

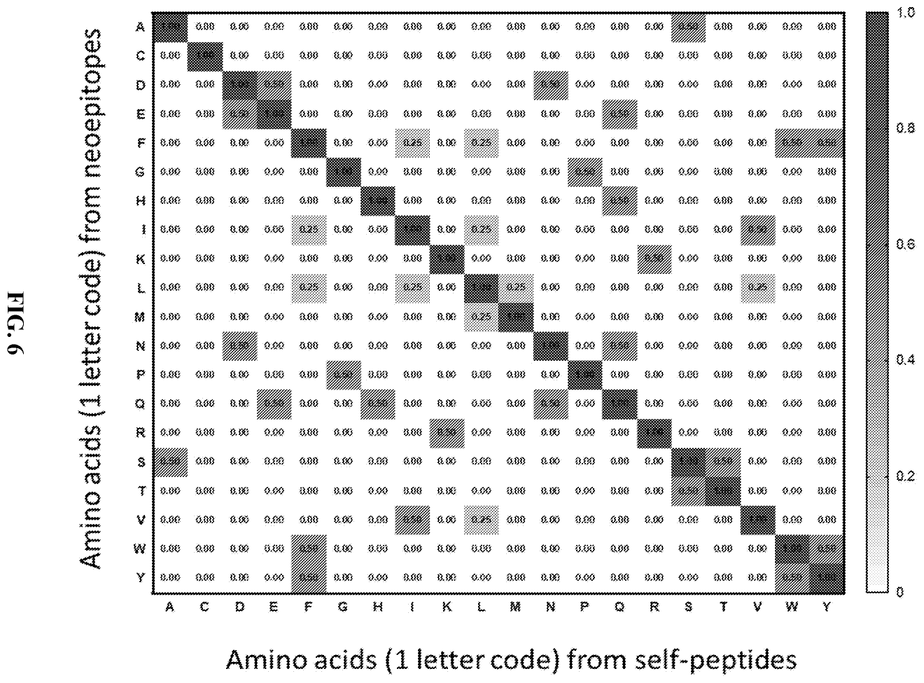

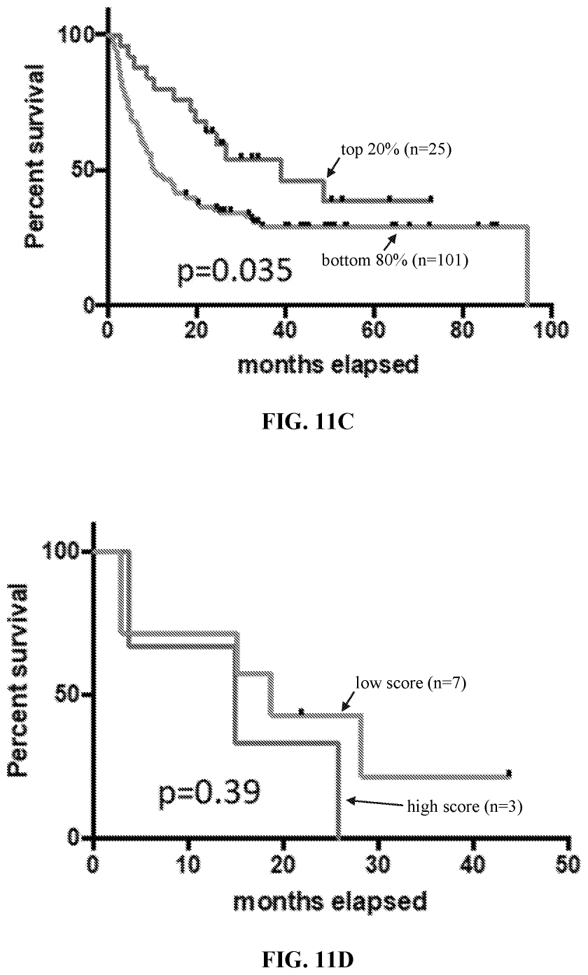

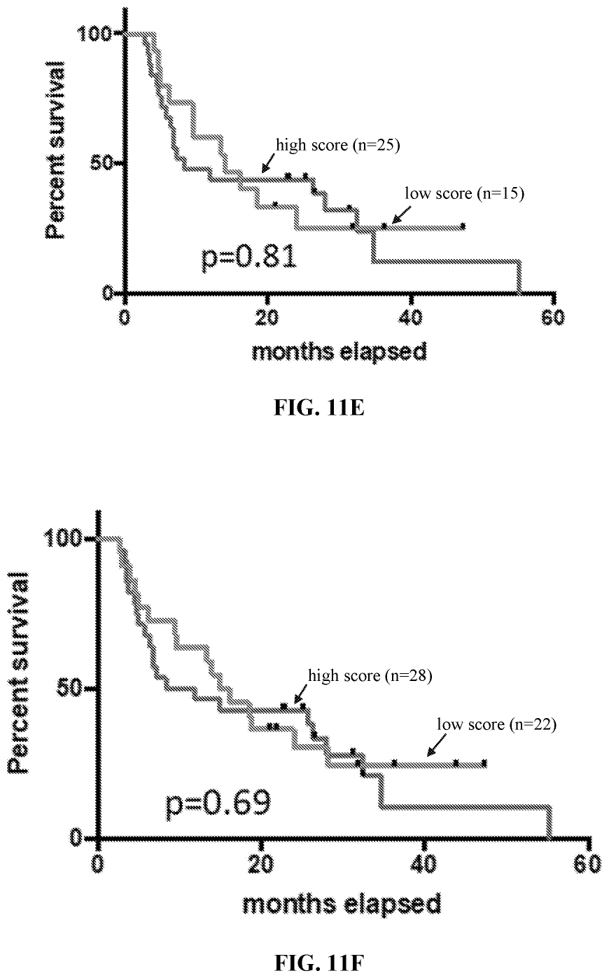

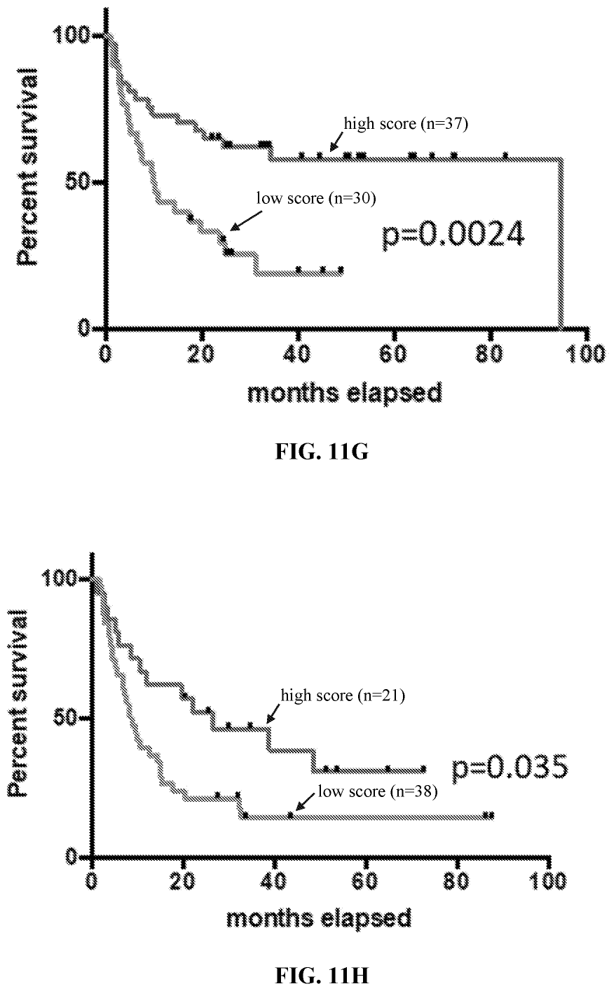

Finally, this example further investigated whether these new insights could be utilized to improve prediction of clinical response to ICB therapies, since tumor mutational burden (TMB) has been shown to be a good predictive biomarker for response to ICB. However, TMB does not take into account the effect of the large number of non-immunogenic mutations. Accordingly, to improve prediction of response to ICB therapy, we developed the RESPONDER score, which is defined as the sum of the so called neoepitope score and the clonality score. Both scores are described in more detail in the methods section. In brief, the neoepitope score is the number of immunogenic neoepitopes in a tumor after eliminating non-immunogenic neoepitopes that were identified through our previously described algorithm. The possibility of an individual neoepitope to be displayed by multiple HLA alleles in the patient and hereby to be presented in higher numbers on the cell surface or to be recognized by multiple T cell clones, is addressed by the clonality score. Three datasets of predicted 9mer neoepitopes based on patients' whole exome sequencing data from a recent survival prediction approach (one NSCLC cohort and two melanoma cohorts)′ were retrieved and the neoepitope and clonality score was applied to the datasets after sorting out those patients showing characteristics associated with either no clear benefit from ICB over chemotherapy (never smokers in NSCLC^{44,45} and PD-L1 negative tumors in NSCLC^{46,47}) or for whom the effect of a biomarker is controversial (NRAS mutated melanoma^{48,49}). Interestingly, each neoepitope and clonality score independently was able to define three subgroups with distinct overall survival rates. The differences between subgroups were highly significant for the neoepitope score (p=0.0002) and there was also a trend for distinguishing the subgroups based on the clonality score (p=0.056; FIGS. 5A-5B). This information was used to define weighted scores by assigning either 1, 2 or 3 points to the subgroups of the clonality score as well as 2, 4 or 6 points for subgroups of the neoepitope score and added the results to calculate the RESPONDER score. The rationale for the double weighted neoepitope score comes from the lower p value in distinguishing the subgroups. When the RESPONDER score was applied to the complete dataset of 148 patients with a score of 7 and above defining high scores, good and poor response subgroups were identified with unprecedented precision (p=2.9×10⁻⁶; FIG. 5C) and higher accuracy compared to more established biomarkers, like tumor mutation burden (FIG. 5D). Also, the RESPONDER score was predictive for both, NSCLC and melanoma, individually (FIGS. 5E-5F). Of note, confidence in the stratification of good and poor responders for NSCLC could be improved 4.5-fold by adjusting the neoepitope score thresholds to account for the different mutational loads in NSCLC compared to melanoma (FIG. 11A). Furthermore, the RESPONDER score again exhibited much higher predictive accuracy than classical non-synonymous mutational burden for both, NSCLC and melanoma subgroups (FIG. 11B-11C). When the RESPONDER score was used to assess the previously excluded subgroups for whom the effect of ICB over chemotherapy is either absent or not clear (never smokers, PD-L1 negative tumors, NRAS mutated patients), the RESPONDER score was not predictive of response (FIGS. 11D-11F). Though no direct conclusion about the biological mechanism can be drawn, these data might suggest that NRAS mutations because of their potency as oncogenic drivers neutralize the effect of T cell responses to neoepitopes. In contrast, when applied to the BRAF-mutated or NRAS/BRAF wild-type subgroups, the RESPONDER score remains highly predictive (FIGS. 11G-1111).

Recently, it has become evident that immunogenic neoepitopes are crucial for the efficacy of many T cell-based therapies, especially checkpoint blockade, TIL treatments, and neoepitope-based vaccination strategies. Although, models have been developed to predict immunogenicity of neoepitopes^{50,51} and response to checkpoint inhibition based on a patient's neoepitope repertoire⁴³, it is still not possible to a priori predict the non-immunogenicity of a specific neoepitope with reasonable certainty. In this example, a model was designed that successfully predicted tolerance to single point-mutated 9mer neoepitopes with high statistical significance in one third of all non-immunogenic neoepitopes tested. Without being bound by theory, this approach provides a novel immunological concept, in which a specific TCR restriction can be circumvented if: 1) the peptide sequence in the TCR recognition area and 2) the absolute affinity of a peptide to its presenting HLA complex, are similar between the neoepitope and the non-mutated HLA ligand. This concept is termed “allelic cross tolerance”. However, even if no allelic cross tolerance is assumed, the model retains specificity and positive predictive value to a highly significant level (Fisher's exact test p=0.0041, FIG. 8B). Nevertheless, the idea of allelic cross tolerance is supported by the initial model, in which the p-value for Fisher's exact test is at least 400 times lower (for Chi-Square tests 120,000 times lower) and sensitivity for identification of non-immunogenic neoepitopes is 3-times higher compared to the models which do not account for allelic cross tolerance. Importantly, the idea of cross-tolerizing HLA alleles might also explain the phenomenon of inconsistent immunogenicity of epitopes between individuals.

In addition to developing this new predictive model, a large number of previously unreported 9mer HLA ligands was identified, which expanded the IEDB database in this category by 25%. This model introduces new criteria for the selection of immunogenic neoepitopes, such as identification of wild-type sequence by mass spectrometry as well as substantial changes in hydrophobicity and volume of point-mutated amino acids, including R to C and T to I.

In a final step, the model's new insights about allelic cross tolerance were used to define the RESPONDER score as a tool for prediction of response to ICB. Retrospectively the RESPONDER score was able to distinguish good and poor response subgroups to ICB with unprecedented precision outperforming tumor mutational load as an alternative biomarker. The RESPONDER score can thus be used for predicting response to ICB solely based on patients' immunogenetic data.

Overall, this example provides a new approach for the prospective prediction of pre-existing tolerance to HLA class I neoepitopes that can be used for improved selection of neoepitopes for clinical studies, aids in the design of faster, small trials and forms the basis for the RESPONDER scoring system which has the ability to predict the survival in response to immune checkpoint blockade in an unprecedented manner, thus sparing many patients from a toxic and ineffective therapy.

Methods

HLA ligand data acquisition. First, HLA ligands were retrieved from IEDB database. In addition to the default setup organism was set to “Homo sapiens, ID:9606”, host to “Humans” and MHC restriction to “MHC Class I”. For the assay selection “Positive Assays Only” and “MHC Ligand Assays” were enabled. Results were filtered after downloading for 9mer peptides. Data cutoff was Sep. 20, 2018. Second, supplementary tables with MS-identified HLA ligands from three studies (Bassani-Sternberg et al., MCP 2015¹⁴; Chong et al., MCP 2018¹⁹ and Bassani-Sternberg et al., Nat Commun 2016²⁰) were downloaded and 9mer HLA ligands extracted.

Mass spectrometry RAW data acquisition. 162 RAW data files were downloaded from PRIDE²⁵ archive. They were retrieved from datasets with the identifiers PXD000394, PXD004894 and PXD006939.

Mass spectrometry data processing. Mass spectrometry data was processed using Byonic software (version 2.7.84, Protein Metrics, Palo Alto, Calif.) through a custom-built computer server equipped with 4 Intel Xeon E5-4620 8-core CPUs operating at 2.2 GHz, and 512 GB physical memory (Exxact Corporation, Freemont, Calif.). Mass accuracy for MS1 was set to 10 ppm and to 20 ppm for MS2, respectively. Digestion specificity was defined as unspecific and only precursors with charges 1, 2, and 3 and up to 2 kDa were allowed. Protein FDR was disabled to allow complete assessment of potential peptide identifications. Oxidization of methionine and N-terminal acetylation were set as variable modifications for all samples. All samples were searched against UniProt Human Reviewed Database (20,349 entries, http://www.uniprot.org, downloaded June 2017).

HLA ligand selection strategy and HLA allele assignment. Peptides annotated by Byonic were further filtered for peptides of 8 to 12 amino acids in length. Duplicates were removed and only identifications with a peptide log prob of 2.0 and higher were accepted representing a p-value for individual peptide spectrum matches of 0.01 or lower. For the prediction model only peptide identifications of 9 amino acids in length were used.

Neoepitope data acquisition and characterization. 14 different studies were used for providing the neoepitope datasets. The following information about the neoepitopes had to be available to be included in the analysis: peptide length and sequence, amino acid change after point-mutation, assigned HLA allele and T cell reactivity based on either ELISpot or multimer assay experiments performed by the reporting studies. Subsequently, predictions for absolute affinity as well as % ranks to the HLA complexes expressed by the patient harboring the neoepitope were calculated by netMHCpan 4.0 to ensure comparability between different neoepitope studies and with unmutated HLA ligands.

Definition of physicochemical similarity among amino acids. A scoring matrix for the physicochemical similarity between two amino acids was defined based on the studies of Kyte³⁹, Zamyatnin⁴⁰ and Pommié et al.⁴². Identical amino acids were set to 1, similarity between amino acids with clear positive (arginine and lysine) or negative charge (aspartic and glutamic acid), all aromatic amino acids (phenylalanine, tyrosine and tryptophan) and all amino acids with amide (asparagine and glutamine) or hydroxyl groups (serine and threonine) in their side chains were set to 0.5. Furthermore, amino acids with almost identical volume (less than 10 Å³ difference) were also assigned to a similarity value of 0.5: alanine to serine, aspartic acid to asparagine, glutamic acid to glutamine and histidine to glutamine. Exemptions from this rule are leucine to isoleucine because of the aliphatic compared to a branched-chain side chain and leucine to methionine because of the special role of the sulfur atom in methionine. Therefore, both amino acids pairs were set to 0.25 instead of 0.5. For amino acids with side chains exclusively built from carbon and hydrogen atoms and differences in volume of less than 30 Å³ similarity was defined by hydropathy index and set to 0.5 for phenylalanine to valine and to 0.25 for phenylalanine to leucine as well as leucine to valine. Finally, one pair of amino acids whose similarity cannot be explained easily by their structure was proline to glycine. The rationale for their similarity comes from experiments defining the binding characteristics of TCR mimic antibodies performed in our lab (data not published). Their similarity score was defined as 0.5.

Prediction of non-immunogenic neoepitopes. A training dataset consisting of 92 (21 immunogenic and 71 non-immunogenic) neoepitopes was defined based on three studies (Ott et al., Nature 2017⁶, Bassani-Sternberg et al., Nat Commun 2016²⁰ and Tanyi et al. Sci Transl Med 2018³⁴). Then, a three-step prediction model for tolerance against neoepitopes was developed: First, the 9mer neoepitope of interest was matched for similarity at positions 4 to 8 with the complete dataset of 169,302 unmutated 9mer HLA ligands. The minimal requirements for a positive match between a neoepitope and an unmutated HLA ligand were defined as: identical amino acids at positions 4, 5 and 8 (each with a score of 1) and at least similar amino acids at positions 6 and 7 based on the scoring matrix in FIG. 6. The combined score of positions 4 to 8 had to reach a minimum of 4.0 though a minimal score of 0.25 was required for positions 6 and 7. Second, the predicted absolute affinities or affinity % ranks for the matching peptide compared to the neoepitope had to fall into a specific range. The range was defined by values 5-times higher or lower as the neoepitopes' affinity or % rank (if the neoepitope could be assigned to multiple HLA alleles of the patient's HLA typing the values for the best scoring allele were used). If the neoepitope and the matching unmutated HLA ligand could be presented on the same HLA complex, absolute affinities were used for comparison. If the neoepitope and the matching HLA ligand were displayed on different HLA complexes, % rank range was used for better comparison between multiple HLA alleles. In a third step, expression patterns of genes which encoded the sequence for a matching HLA ligand were checked at UniProt database. If the gene was exclusively or mostly expressed at immune-privileged sites (eyes, testes, central nervous system, and hair follicles), the matching peptide was discarded since those genes often give rise to immunogenic HLA ligands themselves. Finally, our model was applied to a test dataset consisting of the remaining 345 neoepitopes derived from 11 studies to prospectively test the prediction model.

Prediction of response to immune checkpoint blockade via RESPONDER score. Data about patient specific predicted 9mer neoepitopes as well as survival data for 198 patients was retrieved from Luksza et al., Nature 2017⁴³. Additional clinical information about PD-L1 and smoking status as well as mutational status on NRAS and BRAF was provided by the original publications^{9,33,52}. Automated prediction of non-immunogenic neoepitopes was carried out for each patient individually according to the criteria described in the “prediction of non-immunogenic neoepitopes” section above and results per patient merged. To ensure high confidence in binding of the neoepitopes and unmutated HLA ligands % rank (for a peptide to be considered to be presented was set to 2.5 instead of 4.0 and only % ranks, but not absolute affinity was used to determine a neoepitope match to achieve better interallelic comparability.

Neoepitope score, clonality score, and RESPONDER score were calculated as follows and calculations are exemplified by numbers indicated in square brackets matching the actual data of patient AL4602: First, predicted 9-mer neoepitopes [n=138] were matched for tolerant peptides as described above. Neoepitopes that were according to our model predicted to be non-immunogenic [n=39] were subtracted from the total number of predicted neoepitopes and remaining neoepitopes were defined as “potentially immunogenic neoepitopes (PINs)” [138−39=99].

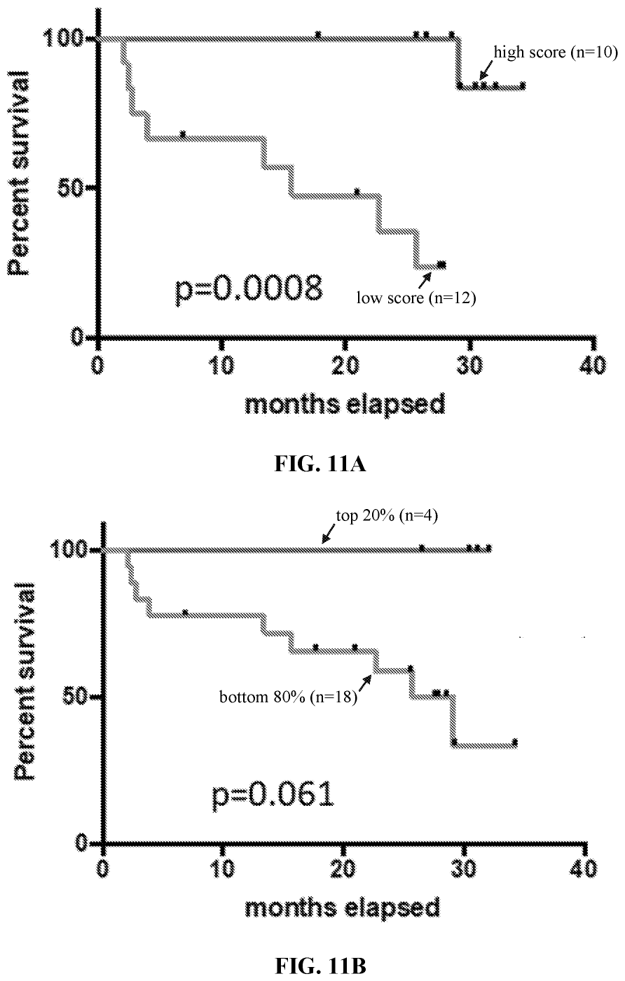

To calculate the final scores, one assumption was adopted from the concept of allelic cross tolerance: If one peptide can be presented on multiple HLA alleles (with a % rank≤2.5), relative affinities to HLA complexes as determined by % ranks were calculated and all peptide:HLA complexes falling into a 5-fold range for % rank affinity are considered one unique peptide:HLA complex. Every unique peptide:HLA complex would then be targeted only by a single T cell clone (See detailed explanation in FIGS. 10A-10C). For example, for patient AL4602, who expresses HLA HLA-A03:01, HLA-A32:01, HLA-B08:01, HLA-B15:01, HLA-007:02, and HLA-C15:02,³³ the neoepitope ATGFQSMVI (SEQ ID NO: 345) would give rise to 2 PINs with % ranks of 1.15 and 0.51. The number of unique peptide:HLA complexes for this neoepitope would be 1 since the % ranks lie within a 5-fold range. In another example the neoepitope FTNRFKIPI (SEQ ID NO: 346) from the same patient would have 4 PINs (% ranks of 0.06, 0.51, 1.92 and 2.26) and therefore 2 unique peptide:HLA complexes.

If then, unique peptide:HLA complexes are determined for every PIN in a patient and the resulting numbers are added, the sum defines the “neoepitope score” [n=79 for AL4602].

The clonality score is calculated as the quotient of neoepitope score [79] over the amount of “potentially immunogenic neoepitopes [99]” or [79/99=0.798]. Because the number of PINs will always be ≤neoepitope score, the resulting clonality score is always ≤1.0. Examples for different clonality scores are illustrated in FIGS. 10A-10C: If a neoepitope can be presented with highly distinct affinities on several HLA alleles of a patient, a high clonality score will be achieved since this mutation can be targeted by multiple T cell clones (FIG. 10A). However, for this model lowest survival rates were observed which may be due to the resulting low numbers of presented peptide:HLA complexes to each T cell clone. This is supported by previous work of our lab that demonstrates that even highly immunogenic epitopes cannot be recognized by T cells if they are presented at low frequency within a tumor⁵³. In reverse, if a neoepitope can only be presented with very similar affinities (within 5-fold % rank range) on multiple HLA alleles only one T cell clone would be specific to this mutation and the clonality score will be the low (FIG. 10B). For this instance, this T cell clone would see more of its target since the neoepitope is displayed by multiple HLA alleles and results in intermediate survival rates. Interestingly, best survival is observed in cases between both extremes, in which neoepitopes are targeted by multiple T cell clones, but are also displayed in higher frequencies (FIG. 10C). Overall, the clonality score describes the ability of a neoepitope to be recognized by higher or lower numbers of T cell clones.

Thresholds for points assigned to both scores are defined as follows:

| Neoepitope | | Clonality | |
|---|---|---|---|
| score | Points | Score | Points |
| >200 | 6 | 0.70 < x ≤ 0.84 | 3 |
| 50 < x ≤ 200 | 4 | ≤0.70 | 2 |
| ≤50 | 2 | >0.84 | 1 |

RESPONDER score=Neoepitope score+Clonality Score.

RESPONDER scores of 7 and above are considered high scores; scores 6 and below low scores.

Graphs and statistics. All graphs were drawn with Graphpad Prism 7. Statistical analyses were mostly performed by Graphpad Prism 7, Fisher's exact test was calculated by the online tool https://www.socscistatistics.com/tests/fisher/Default2.aspx. P-values from Chi-Square results were calculated using the web platform http://courses.atlas.illinois.edu/fall2017/STAT/STAT200/pchisq.html.

### REFERENCES

The references cited throughout this application are listed below. Each reference is incorporated by reference in their entirety.
1. Gubin, M. M., et al. Checkpoint blockade cancer immunotherapy targets tumour-specific mutant antigens. Nature 515, 577-581 (2014).
2. Schumacher, T., et al. A vaccine targeting mutant IDH1 induces antitumour immunity. Nature 512, 324-327 (2014).
3. Karpanen, T. & Olweus, J. The Potential of Donor T-Cell Repertoires in Neoantigen-Targeted Cancer Immunotherapy. Front Immunol 8, 1718 (2017).
4. Hilf, N., et al. Actively personalized vaccination trial for newly diagnosed glioblastoma. Nature (2018).
5. Keskin, D. B., et al. Neoantigen vaccine generates intratumoral T cell responses in phase Ib glioblastoma trial. Nature (2018).
6. Ott, P. A., et al. An immunogenic personal neoantigen vaccine for patients with melanoma. Nature 547, 217-221 (2017).
7. Sahin, U., et al. Personalized RNA mutanome vaccines mobilize poly-specific therapeutic immunity against cancer. Nature 547, 222-226 (2017).
8. De Henau, O., et al. Overcoming resistance to checkpoint blockade therapy by targeting PI3Kgamma in myeloid cells. Nature 539, 443-447 (2016).
9. Van Allen, E. M., et al. Genomic correlates of response to CTLA-4 blockade in metastatic melanoma. Science 350, 207-211 (2015).
10. Gopalakrishnan, V., et al. Gut microbiome modulates response to anti-PD-1 immunotherapy in melanoma patients. Science 359, 97-103 (2018).
11. Sivan, A., et al. Commensal Bifidobacterium promotes antitumor immunity and facilitates anti-PD-L1 efficacy. Science 350, 1084-1089 (2015).
12. Stronen, E., et al. Targeting of cancer neoantigens with donor-derived T cell receptor repertoires. Science 352, 1337-1341 (2016).
13. Samstein, R. M., et al. Tumor mutational load predicts survival after immunotherapy across multiple cancer types. Nat Genet 51, 202-206 (2019).
14. Lorenz, R G & Allen, P M. Thymic cortical epithelial cells can present self-antigens in vivo. Nature 337, 560-562 (1989).
15. Bassani-Sternberg, M., Pletscher-Frankild, S., Jensen, L. J. & Mann, M. Mass spectrometry of human leukocyte antigen class I peptidomes reveals strong effects of protein abundance and turnover on antigen presentation. Mol Cell Proteomics 14, 658-673 (2015).
16. Hu, Q., et al. The Orbitrap: a new mass spectrometer. J Mass Spectrom 40, 430-443 (2005).
17. Ma, B., et al. PEAKS: powerful software for peptide de novo sequencing by tandem mass spectrometry. Rapid Commun Mass Spectrom 17, 2337-2342 (2003).
18. Eng, J. K., McCormack, A. L. & Yates, J. R. An approach to correlate tandem mass spectral data of peptides with amino acid sequences in a protein database. J Am Soc Mass Spectrom 5, 976-989 (1994).
19. Cox, J. & Mann, M. MaxQuant enables high peptide identification rates, individualized p.p.b.-range mass accuracies and proteome-wide protein quantification. Nat Biotechnol 26, 1367-1372 (2008).
20. Chong, C., et al. High-throughput and Sensitive Immunopeptidomics Platform Reveals Profound Interferongamma-Mediated Remodeling of the Human Leukocyte Antigen (HLA) Ligandome. Mol Cell Proteomics 17, 533-548 (2018).
21. Bassani-Sternberg, M., et al. Direct identification of clinically relevant neoepitopes presented on native human melanoma tissue by mass spectrometry. Nat Commun 7, 13404 (2016).
22. Jurtz, V., et al. NetMHCpan-4.0: Improved Peptide-MHC Class I Interaction Predictions Integrating Eluted Ligand and Peptide Binding Affinity Data. J Immunol 199, 3360-3368 (2017).
23. Abelin, J. G., et al. Mass Spectrometry Profiling of HLA-Associated Peptidomes in Mono-allelic Cells Enables More Accurate Epitope Prediction. Immunity 46, 315-326 (2017).
24. Vita, R., et al. The Immune Epitope Database (IEDB): 2018 update. Nucleic Acids Res (2018).
25. Bern, M., Kil, Y. J. & Becker, C. Byonic: advanced peptide and protein identification software. Curr Protoc Bioinformatics Chapter 13, Unit13 20 (2012).
26. Reisinger, F., del-Toro, N., Ternent, T., Hermjakob, H. & Vizcaino, J. A. Introducing the PRIDE Archive RESTful web services. Nucleic Acids Res 43, W599-604 (2015).
27. Wick, D. A., et al. Surveillance of the tumor mutanome by T cells during progression from primary to recurrent ovarian cancer. Clin Cancer Res 20, 1125-1134 (2014).
28. Tran, E., et al. Immunogenicity of somatic mutations in human gastrointestinal cancers. Science 350, 1387-1390 (2015).
29. Cohen, C. J., et al. Isolation of neoantigen-specific T cells from tumor and peripheral lymphocytes. J Clin Invest 125, 3981-3991 (2015).
30. Gros, A., et al. Prospective identification of neoantigen-specific lymphocytes in the peripheral blood of melanoma patients. Nat Med 22, 433-438 (2016).
31. Prickett, T. D., et al. Durable Complete Response from Metastatic Melanoma after Transfer of Autologous T Cells Recognizing 10 Mutated Tumor Antigens. Cancer Immunol Res 4, 669-678 (2016).
32. Bentzen, A. K., et al. Large-scale detection of antigen-specific T cells using peptide-MHC-I multimers labeled with DNA barcodes. Nat Biotechnol 34, 1037-1045 (2016).
33. Rizvi, N. A., et al. Cancer immunology. Mutational landscape determines sensitivity to PD-1 blockade in non-small cell lung cancer. Science 348, 124-128 (2015).
34. Li, F., et al. Rapid tumor regression in an Asian lung cancer patient following personalized neo-epitope peptide vaccination. Oncoimmunology 5, e1238539 (2016).
35. Tanyi, J. L., et al. Personalized cancer vaccine effectively mobilizes antitumor T cell immunity in ovarian cancer. Sci Transl Med 10(2018).
36. Carreno, B. M., et al. Cancer immunotherapy. A dendritic cell vaccine increases the breadth and diversity of melanoma neoantigen-specific T cells. Science 348, 803-808 (2015).
37. Engels, B., et al. Relapse or eradication of cancer is predicted by peptide-major histocompatibility complex affinity. Cancer Cell 23, 516-526 (2013).
38. Chowell, D., et al. TCR contact residue hydrophobicity is a hallmark of immunogenic CD8+ T cell epitopes. Proc Natl Acad Sci USA 112, E1754-1762 (2015).
39. Kyte, J. & Doolittle, R. F. A simple method for displaying the hydropathic character of a protein. J Mol Biol 157, 105-132 (1982).
40. Zamyatnin, A. A. Protein volume in solution. Prog Biophys Mol Biol 24, 107-123 (1972).
41. Calis, J. J., de Boer, R. J. & Kesmir, C. Degenerate T-cell recognition of peptides on MHC molecules creates large holes in the T-cell repertoire. PLoS Comput Biol 8, e1002412 (2012).
42. Pommie, C., Levadoux, S., Sabatier, R., Lefranc, G. & Lefranc, M. P. IMGT standardized criteria for statistical analysis of immunoglobulin V-REGION amino acid properties. J Mol Recognit 17, 17-32 (2004).
43. Luksza, M., et al. A neoantigen fitness model predicts tumour response to checkpoint blockade immunotherapy. Nature 551, 517-520 (2017).
44. Kim, J. H., Kim, H. S. & Kim, B. J. Prognostic value of smoking status in non-small-cell lung cancer patients treated with immune checkpoint inhibitors: a meta-analysis. Oncotarget 8, 93149-93155 (2017).
45. Li, B., Huang, X. & Fu, L. Impact of smoking on efficacy of PD-1/PD-L1 inhibitors in non-small cell lung cancer patients: a meta-analysis. Onco Targets Ther 11, 3691-3696 (2018).
46. Abdel-Rahman, O. Correlation between PD-L1 expression and outcome of NSCLC patients treated with anti-PD-1/PD-L1 agents: A meta-analysis. Crit Rev Oncol Hematol 101, 75-85 (2016).
47. Passiglia, F., et al. PD-L1 expression as predictive biomarker in patients with NSCLC: a pooled analysis. Oncotarget 7, 19738-19747 (2016).
48. Johnson, D. B., et al. Impact of NRAS mutations for patients with advanced melanoma treated with immune therapies. Cancer Immunol Res 3, 288-295 (2015).
49. Kirchberger, M. C., et al. MEK inhibition may increase survival of NRAS-mutated melanoma patients treated with checkpoint blockade: Results of a retrospective multicentre analysis of 364 patients. Eur J Cancer 98, 10-16 (2018).
50. Bjerregaard, A. M., et al. An Analysis of Natural T Cell Responses to Predicted Tumor Neoepitopes. Front Immunol 8, 1566 (2017).
51. Kosaloglu-Yalcin, Z., et al. Predicting T cell recognition of MHC class I restricted neoepitopes. Oncoimmunology 7, e1492508 (2018).
52. Snyder, A., et al. Genetic basis for clinical response to CTLA-4 blockade in melanoma. N Engl J Med 371, 2189-2199 (2014).
53. Gejman, R. S., et al. Rejection of immunogenic tumor clones is limited by clonal fraction. Elife 7 (2018).

TABLE 2
Collection of neoepitopes and matches from unmutated HLA ligands

| | | presenting | | | |
|---|---|---|---|---|---|
| neoepitope | | HLA | uniprot | Gene | Protein name of |
| identifier | Sequence | allele* | identifier⁺ | name⁺ | HLA-LM⁺ |
| Bassani- | ETSKQVTRW (SEQ ID | A25 | | | |
| Sternberg_5 | NO: 6) | | | | |
| | SLKKQLTRV (SEQ ID | B08 | O95602 | POLR1A | DNA-directed RNA |
| | NO: 7) | | | | polymerase I subunit |
| | | | | | RPA1 |
| Ott_6 | TELERFLEY (SEQ ID | B4402 | | | Serine/threonine- |
| | NO: 8) | | | | protein phosphatase 6 |
| | QLIERILEA (SEQ ID | A02 | Q5H9R7 | PPP6R3 | regulatory subunit 3 |
| | NO: 9) | | | | |
| Ott_7 | LLHTELERF (SEQ ID | B15 | | | |
| | NO: 10) | | | | |
| | YLRTELERL (SEQ ID | A02 | Q5VUA4 | ZNF318 | Zinc finger protein |
| | NO: 11) | | | | 318 |
| Ott_8 | TLFHTFYEL (SEQ ID | A02 | | | |
| | NO: 12) | | | | |
| | VYHHTFFEM (SEQ ID | A24 | P49588 | AARS | Alanine--tRNA |
| | NO: 13) | | | | ligase, cytoplasmic |
| | SLLHTIYEV (SEQ ID | A02 | Q969G9 | NKD1 | Protein naked cuticle |
| | NO: 14) | | | | homolog 1 |
| | SLMHTIYEV (SEQ ID | A02 | Q969F2 | NKD2 | Protein naked cuticle |
| | NO: 15) | | | | homolog 2 |
| Ott_11 | KLFESKAEL (SEQ ID | A02 | O75153 | CLUH | Clustered |
| | NO: 16) | | | | mitochondria protein |
| | RVYESKAEF (SEQ ID | B15 | | | homolog |
| | NO: 17) | | | | |
| | QEAESKSEL (SEQ ID | B4402 | Q9BZH6 | WDR11 | WD repeat-containing |
| | NO: 18) | | | | protein 11 |
| | AEAESRAEA (SEQ ID | B4402 | Q14764 | MVP | Major vault protein |
| | NO: 19) | | | | |
| Ott_13 | GIPENSFNV (SEQ ID | A02 | | | |
| | NO: 20) | | | | |
| | RLPENTFNI (SEQ ID | A24 | Q8IVU3 | HERC6 | Probable E3 |
| | NO: 21) | | | | ubiquitin-protein |
| Ott_25 | NVLSSLVLV (SEQ ID | A02 | | | |
| | NO: 22) | | | | |
| | HLLSSLLLY (SEQ ID | A03 | Q07002 | CDK18 | Cyclin-dependent |
| | NO: 23) | | | | kinase 18 |
| | TGFSSLFLK (SEQ ID | A03 | Q8N201 | INTS1 | Integrator complex |
| | NO: 24) | | | | subunit 1 |
| Ott_26 | RLMLRKVAL (SEQ ID | | | | |
| | A02 | | | | |
| | NO: 25) | | | | |
| | TESLRKIAL (SEQ ID | B47 | Q96NL6 | SCLT1 | Sodium channel and |
| | NO: 26) | | | | clathrin linker 1 |
| Ott_27 | ALQSQSISL (SEQ ID | A02 | | | |
| | NO: 27) | | | | |
| | SQCSQSLSV (SEQ ID | B47 | Q9NVI1 | FANCI | Fanconi anemia group |
| | NO: 28) | | | | I protein |
| Ott_31 | KLNFRLFVI (SEQ ID | A02 | | | |
| | NO: 29) | | | | |
| | SRLFRVFVH (SEQ ID | B2705 | Q96BX8 | MOB3A | MOB kinase activator |
| | NO: 30) | | | | 3A |
| Ott-32 | FEAEFTQVA (SEQ ID | B18 | | | |
| | NO: 31) | | | | |
| | FAAEFSNVM (SEQ ID | A25 | Q9UDY8 | MALT1 | Mucosa-associated |
| | NO: 32) | | | | lymphoid tissue |
| | | | | | lymphoma |
| | | | | | translocation protein 1 |
| Ott_38 | WLVDLLPST (SEQ ID | A02 | | | |
| | NO: 33) | | | | |
| | SVDDLLPSL (SEQ ID | A02 | Q14289 | PTK2B | Protein-tyrosine |
| | NO: 34) | | | | kinase 2-beta |
| | DLIDLVPSL (SEQ ID | A25 | P47756-2 | CAPZB | F-actin-capping |
| | NO: 35) | | | | protein subunit beta |
| | SRIDLIPSL (SEQ ID | B2702 | Q99567 | Nup88 | Nuclear pore complex |
| | NO: 36) | | | | protein Nup88 |
| Ott_45 | REFDKIELA (SEQ ID | B41 | | | |
| | NO: 37) | | | | |
| | TAVDKVELF (SEQ ID | B35 | Q14511 | NEDD1 | Enhancer of |
| | NO: 38) | | | | filamentation 1 |
| | AEVDKLELM (SEQ ID | B41 | Q8WVK7 | SKA2 | Spindle and |
| | NO: 39) | | | | kinetochore- |
| | | | | | associated protein 2 |
| Ott_53 | ALPQSILLF (SEQ ID | A23 | | | |
| | NO: 40) | | | | |
| | RQDQSIILL (SEQ ID | B41 | Q92614 | MY018A | Unconventional |
| | NO: 41) | | | | myosin-XVIIIa |
| | RVDQSLLLY (SEQ ID | B35 | Q67FW5 | B3GNTL1 | UDP- |
| | NO: 42) | | | | GlcNAc:betaGal beta- |
| | | | | | 1,3-N- |
| | | | | | acetylglucosaminyltra |
| | | | | | nsferase-like protein 1 |
| Ott_56 | TIIDNIKEM (SEQ ID | A66 | | | |
| | NO: 43) | | | | |
| | YGYDNVKEY (SEQ ID | B35 | Q96GN5 | CDCA7L | Cell division cycle- |
| | NO: 44) | | | | associated 7-like protein |
| Ott_66 | TSIQSPSLY (SEQ ID | A01 | | | |
| | NO: 45) | | | | |
| | RTAQSGALR (SEQ ID | A66 | P40222 | TXLNA | Alpha-taxilin |
| | NO: 46) | | | | |
| Ott_67 | HLARHRHLM (SEQ ID | B08 | | | |
| | NO: 47) | | | | |
| | FVFRHKQLL (SEQ ID | B08 | Q9NYV6 | RRN3 | RNA polymerase |
| | NO: 48) | | | | I-specific transcription |
| | | | | | initiation factor RRN3 |
| Ott_70 | HTLGAASSF (SEQ ID | A66 | | | |
| | NO: 49) | | | | |
| | GSDGAASSY (SEQ ID | A01 | Q14203 | DCTN1 | Dynactin subunit 1 |
| | NO: 50) | | | | |
| Ott_73 | NVELRRNVL (SEQ ID | B08 | | | |
| | NO: 51) | | | | |
| | NPDLRRNVL (SEQ ID | B08 | Q15560 | TCEA2 | Transcription |
| | NO: 52) | | | | elongation factor A |
| | | | | | protein 2 |
| | NPNLRKNVL (SEQ ID | B08 | P23193 | TCEA1 | Transcription |
| | NO: 53) | | | | elongation factor A |
| | | | | | protein 1 |
| Ott_75 | SIKEITNFK (SEQ ID | A66 | | | |
| | NO: 54) | | | | |
| | TVAEISQFL (SEQ ID | A66 | Q9Y689 | ARL5A | ADP-ribosylation |
| | NO: 55) | | | | factor-like protein 5A |
| Ott_76 | ESIKEITNF (SEQ ID | A66 | | | |
| | NO: 56) | | | | |
| | | | | | Myosin light chain |
| | DVRKEVTNV (SEQ ID | A66 | Q15746 | myLK | kinase, smooth |
| | NO: 57) | | | | muscle |
| Wick_3 | FMASNDEGV (SEQ ID | C12 | | | |
| | NO: 58) | | | | |
| | KIISNEEGY (SEQ ID | B35 | P27487 | DPP4 | Dipeptidyl peptidase |
| | NO: 59) | | | | 4 |
| Wick_12 | FLLLVAAMI (SEQ ID | A02 | | | |
| | NO: 60) | | | | |
| | KLSLVAAML (SEQ ID | A02 | P11021 | HSPA5 | Endoplasmic |
| | NO: 61) | | | | reticulum chaperone |
| | | | | | BiP |
| Wick_18 | FQDDDQTRL (SEQ ID | B39 | | | |
| | NO: 62) | | | | |
| | FQDDDQTRV (SEQ ID | A02 | Q9NVH1 | DNAJC11 | DnaJ homolog |
| | NO: 63) | | | | subfamily C member |
| | | | | | 11 |
| Wick_19 | KAIESFLEK (SEQ ID | A30 | | | |
| | NO: 64) | | | | |
| | FTDESYLEL (SEQ ID | C14 | Q01780 | EXOSC10 | Exosome component |
| | NO: 65) | | | | 10 |
| | SASESILEL (SEQ ID | B39 | P42695 | NCAPD3 | Condensin-2 complex |
| | NO: 66) | | | | subunit D3 |
| Wick_22 | KLLMSQANV (SEQ ID | A02 | | | |
| | NO: 67) | | | | |
| | KLVMSQANV (SEQ ID | A02 | Q9H009 | NACA2 | Nascent polypeptide- |
| | NO: 68) | | | | associated complex |
| | | | | | subunit alpha-2 |
| Wick_24 | YTHNLIFVF (SEQ ID | C14 | | | |
| | NO: 69) | | | | |
| | QLNNLVYVV (SEQ ID | A02 | Q8NEC7 | GSTCD | Glutathione S- |
| | NO: 70) | | | | transferase C-terminal |
| | | | | | domain-containing |
| | | | | | protein |
| | | | | | Echinoderm |
| | SHDNLVYVY (SEQ ID | C14 | O95834 | EML2 | microtubule- |
| | NO: 71) | | | | associated |
| | | | | | protein-like 2 |
| Wick_27 | YTAQIILAL (SEQ ID | B39 | | | |
| | NO: 72) | | | | |
| | KTSQIFLAK (SEQ ID | A30 | Q9UPN3 | MACF1 | Microtubule-actin |
| | NO: 73) | | | | cross-linking factor 1, |
| | | | | | isoforms 1/2/3/5 |
| Tran_1 | FGDVGSTLF (SEQ ID | C08 | | | |
| | NO: 74) | | | | |
| | TSDVGATLL (SEQ ID | C08 | Q96AP0 | ACD | Adrenocortical |
| | NO: 75) | | | | dysplasia protein |
| | | | | | homolog |
| Tran_2 | FLKELLVRI (SEQ ID | A02 | | | |
| | NO: 76) | | | | |
| | TMLELLLRL (SEQ ID | A02 | Q13129 | RLF | Zinc finger protein |
| | NO: 77) | | | | Rlf |
| | FPGELLLRL (SEQ ID | B56 | Q15758 | SLC1A5 | Neutral amino acid |
| | NO: 78) | | | | transporter B(0) |
| | ILAELLLRV (SEQ ID | A02 | | | |
| | NO: 79) | | | | |
| Tran_6 | RELVHRILL (SEQ ID | B18 | | | |
| | NO: 80) | | | | |
| | SDMVHRFLL (SEQ ID | B14 | Q9NZ08 | ERAp1 | Endoplasmic |
| | NO: 81) | | | | reticulum |
| | | | | | aminopeptidase 1 |
| | RPYVHKILV (SEQ ID | B14 | O75533 | SF3B1 | Splicing factor 3B |
| | NO: 82) | | | | subunit 1 |
| Stronen_3 | YLVDSVAKM (SEQ ID | A02 | | | |
| | NO: 83) | | | | |
| | YLVDSVAKT (SEQ ID | A02 | P46734 | MAP2K3 | Dual specificity |
| | NO: 84) | | | | mitogen-activated |
| | | | | | protein kinase kinase |
| | | | | | 3 |
| Stronen_6 | SLFALGNVI (SEQ ID | A02 | | | |
| | NO: 85) | | | | |
| | FHLALGQVL (SEQ ID | C03 | P98171 | ARGHAP4 | Rho GTPase- |
| | NO: 86) | | | | activating protein 4 |
| | FALGNVISA (SEQ ID | A02 | | | |
| | NO: 87) | | | | |
| Stronen_8 | MPFGNVISA (SEQ ID | C03 | P95319 | CELF2 | CUGBP Elav-like |
| | NO: 88) | | | | family member 2 |
| | MPFGNVVSA (SEQ ID | C03 | Q92879 | CELF1 | CUGBP Elav-like |
| | NO: 89) | | | | family member 1 |
| Stronen_11 | FLMASISSF (SEQ ID | A02 | | | |
| | NO: 90) | | | | |
| | AVAASISSK (SEQ ID | A11 | P09086 | POU2F2 | POU domain, class 2, |
| | NO: 91) | | | | transcription factor 2 |
| | FLPASVASL (SEQ ID | A02 | O75564 | JRK | Jerky protein |
| | NO: 92) | | | | homolog |
| | SAAASVASR (SEQ ID | A11 | Q9H1B7 | IRF2BPL | Interferon regulatory |
| | NO: 93) | | | | factor 2-binding |
| | | | | | protein-like |
| | EIPASVSSY (SEQ ID | B35 | P98177 | FOXO4 | Forkhead box protein |
| | NO: 94) | | | | O4 |
| | TVPASFSSL (SEQ ID | C07 | Q9H9A6 | LRRC40 | Leucine-rich repeat- |
| | NO: 95) | | | | containing protein 40 |
| | ISAASFSSL (SEQ ID | C07 | Q9NY59 | SMPD3 | Sphingomyelin |
| | NO: 96) | | | | phosphodiesterase 3 |
| Stronen_15 | AQFKGAWIL (SEQ ID | A02 | | | |
| | NO: 97) | | | | |
| | FLPKGAYIY (SEQ ID | B35 | P26639 | TARS | Threonine--tRNA |
| | NO: 98) | | | | ligase, cytoplasmic |
| Stronen_17 | LMASISSFL (SEQ ID | A02 | | | |
| | NO: 99) | | | | |
| | GLTSISTFL (SEQ ID | A02 | Q8TCJ2 | STT3B | Dolichyl- |
| | NO: 100) | | | | diphosphooligosaccha |
| | | | | | ride--protein |
| | | | | | glycosyltransferase |
| | | | | | subunit STT3B |
| | NQASITSFL (SEQ ID | C04 | Q9NR09 | BIRC6 | Baculoviral IAP |
| | NO: 101) | | | | repeat-containing |
| | | | | | protein 6 |
| | IMDSIAAFL (SEQ ID | A02 | Q9BSJ8 | ESYT1 | Extended |
| | NO: 102) | | | | synaptotagmin-1 |
| Stronen_21 | FQPSFSHLV (SEQ ID | A02 | | | |
| | NO: 103) | | | | |
| | FAASFAHLL (SEQ ID | B35 | Q9UKZ1 | CNOT11 | CCR4-NOT |
| | NO: 104) | | | | transcription complex |
| | | | | | subunit 11 |
| Stronen_22 | FLQFRGNEV (SEQ ID | A02 | | | |
| | NO: 105) | | | | |
| | LSSFRGQEF (SEQ ID | B35 | Q2NKX8 | ERCC6L | DNA excision repair |
| | NO: 106) | | | | protein ERCC-6-like |
| | VSSFRPNEF (SEQ ID | C07 | O75815 | BCAR3 | Breast cancer anti- |
| | NO: 107) | | | | estrogen resistance |
| | | | | | protein 3 |
| Stronen_23 | GSLDVLMAV (SEQ ID | A02 | | | |
| | NO: 108) | | | | |
| | SRLDVLLAL (SEQ ID | C04 | O43196 | MSH5 | MutS protein |
| | NO: 109) | | | | homolog 5 |
| | SRLDVLLAL (SEQ ID | C07 | O4319 | MSH5 | MutS protein |
| | NO: 109) | 6 | | | homolog 5 |
| | FAADVLMAI (SEQ ID | A02 | Q9BXK1 | KLF16 | Krueppel-like factor |
| | NO: 110) | | | | 16 |
| | KITDVIMAF (SEQ ID | C07 | P35749 | MYH11 | Myosin-11 |
| | NO: 111) | | | | |
| Stronen_33 | VTYSGKFLI (SEQ ID | A02 | | | |
| | NO: 112) | | | | |
| | LIYSGKLLL (SEQ ID | A02 | Q15011-2 | HERPUD1 | Homocysteine-responsive |
| | NO: 113) | | | | endoplasmic |
| | | | | | reticulum-resident |
| | | | | | ubiquitin-like domain |
| | | | | | member 1 protein |
| | FSKSGRLLL (SEQ ID | B07 | Q9HAV0 | GNB4 | Guanine nucleotide- |
| | NO: 114) | | | | binding protein |
| | | | | | subunit beta-4 |
| | GTWSGRVLV (SEQ ID | A02 | Q9H977 | WDR54 | WD repeat-containing |
| | NO: 115) | | | | protein 54 |
| Rizvi_4 | VTGRLASGK (SEQ ID | A11 | | | |
| | NO: 116) | | | | |
| | VVLRLATGF (SEQ ID | C16 | Q9BQA9 | CYBC1 | Cytochrome b-245 |
| | NO: 117) | | | | chaperone 1 |
| Rizvi_5 | TSDILKIPK (SEQ ID | A11 | | | |
| | NO: 118) | | | | |
| | VPEILRVPL (SEQ ID | B51 | Q7Z478 | DHX29 | ATP-dependent RNA |
| | NO: 119) | | | | helicase DHX29 |
| Rizvi_9 | KHLQVNITL (SEQ ID | C07 | | | |
| | NO: 120) | | | | |
| | RQAQVNLTV (SEQ ID | A02 | Q15746 | MYLK | Myosin light chain |
| | NO: 121) | | | | kinase, smooth |
| | | | | | muscle |
| | RLNQVNVTF (SEQ ID | B18 | P78508 | KCNJ10 | ATP-sensitive inward |
| | NO: 122) | | | | rectifier potassium |
| | | | | | channel 10 |
| Rizvi_15 | TKSSYTWFM (SEQ ID | C07 | | | |
| | NO: 123) | | | | |
| | PAESYTFFI (SEQ ID | B51 | P48556 | PSMD8 | 26S proteasome non- |
| | NO: 124) | | | | ATPase regulatory |
| | | | | | subunit 8 |
| Rizvi_16 | RTLGQAFEV (SEQ ID | A02 | | | |
| | NO: 125) | | | | |
| | STIGQAFEL (SEQ ID | A02 | P29353 | SHC1 | SHC-transforming |
| | NO: 126) | | | | protein 1 |
| Rizvi_17 | STWDSWNER (SEQ ID | A11 | | | |
| | NO: 127) | | | | |
| | KAKDSFNEK (SEQ ID | A11 | Q9NQC3 | RTN4 | Reticulon-4 |
| | NO: 128) | | | | |
| Rizvi_21 | LESPALPMI (SEQ ID | B18 | | | |
| | NO: 129) | | | | |
| | DFDPALGMIVI (SEQ ID | C07 | Q16206 | ENOX2 | Ecto-NOX disulfide- |
| | NO: 130) | | | | thiol exchanger 2 |
| Rizvi_23 | NEAPLILPQ (SEQ ID | B18 | | | |
| | NO: 131) | | | | |
| | SRVPLLLPL (SEQ ID | C07 | Q6EMK4 | VASN | Vasorin |
| | NO: 132) | | | | |
| | LISPLLLPV (SEQ ID | A02 | Q96M86 | DNHD1 | Dynein heavy chain |
| | NO: 133) | | | | domain-containing |
| | | | | | protein 1 |
| | ELFPLIFPA (SEQ ID | A02 | Q04206 | RELA | Transcription factor |
| | NO: 134) | | | | p65 |
| Rizvi_26 | FNMSYKYPI (SEQ ID | C16 | | | |
| | NO: 135) | | | | |
| | DAISYRFPR (SEQ ID | A11 | P78357 | CNTNAP1 | Contactin-associated |
| | NO: 136) | | | | protein 1 |
| | DAISYRFPR (SEQ ID | B18 | P78357 | CNTNAP1 | Contactin-associated |
| | NO: 136) | | | | protein 1 |
| Rizvi_31 | GLQSFQMLV (SEQ ID | A02 | | | |
| | NO: 137) | | | | |
| | LVNSFQLLY (SEQ ID | A11 | Q14739 | LBR | Lamin-B receptor |
| | NO: 138) | | | | |
| Rizvi_34 | SNHDLIQRL (SEQ ID | C07 | | | |
| | NO: 139) | | | | |
| | KLNDLIQRL (SEQ ID | C07 | P53621 | COPA | Coatomer subunit |
| | NO: 140) | | | | alpha |
| | MVKDLINRM (SEQ ID | C07 | Q00341 | HDLBP | Vigilin |
| | NO: 141) | | | | |
| | QTYDLIERR (SEQ ID | A11 | Q12789 | GTF3C1 | General transcription |
| | NO: 142) | | | | factor 3C polypeptide 1 |
| | AIYDLIERI (SEQ ID | A02 | Q96P47 | AGAP3 | Arf-GAP with |
| | NO: 143) | | | | GTPase, ANK repeat |
| | | | | | and PH domain- |
| | | | | | containing protein 3 |
| | GEFDLVQRI (SEQ ID | B18 | Q13625 | TP53BP2 | Apoptosis-stimulating |
| | NO: 144) | | | | of p53 protein 2 |
| Rizvi_37 | ASLETGFAK (SEQ ID | A11 | | | |
| | NO: 145) | | | | |
| | ASVETGFAK (SEQ ID | A11 | Q9BRQ8 | AIFM2 | Apoptosis-inducing |
| | NO: 146) | | | | factor 2 |
| Rizvi_38 | SLETGFAKK (SEQ ID | A11 | | | |
| | NO: 147) | | | | |
| | LEHTGFSKA (SEQ ID | B18 | P48200 | IREB2 | Iron-responsive |
| | NO: 148) | | | | element-binding |
| | | | | | protein 2 |
| Rizvi_42 | LEAAGLLTY (SEQ ID | B18 | | | |
| | NO: 149) | | | | |
| | ALWAGLLTL (SEQ ID | A02 | P06734 | FCER2 | Low affinity |
| | NO: 150) | | | | immunoglobulin |
| | | | | | epsilon Fc receptor |
| | KSYAGFLTV (SEQ ID | C16 | Q9H3G5 | CPVL | Probable serine |
| | NO: 151) | | | | carboxypeptidase |
| | | | | | CPVL |
| Rizvi_44 | LIVMFPFLL (SEQ ID | A02 | | | |
| | NO: 152) | | | | |
| | MVKMFPLLV (SEQ ID | A02 | Q5149U9 | DDX6OL | Probable ATP-dependent |
| | NO: 153) | | | | RNA helicase DDX60-like |
| Rizvi_46 | VMFPFLLIL (SEQ ID | A02 | | | |
| | NO: 154) | | | | |
| | ILIPFMLIL (SEQ ID | A02 | Q8NH06 | OR1P1 | Olfactory receptor |
| | NO: 155) | | | | 1P1 |
| Rizvi_48 | IEHEHLNQY (SEQ ID | B18 | | | |
| | NO: 156) | | | | |
| | LPVEHVNQL (SEQ ID | B51 | Q8IY145 | ZZZ3 | ZZ-type zinc finger- |
| | NO: 157) | | | | containing protein 3 |
| Rizvi_57 | RLQEAVEAA (SEQ ID | A02 | | | |
| | NO: 158) | | | | |
| | SLQEAVQAA (SEQ ID | A02 | Q15274 | QPRT | Nicotinate-nucleotide |
| | NO: 159) | | | | pyrophosphorylase |
| | | | | | [carboxylating] |
| | HLIEAVEAI (SEQ ID | A02 | Q9H2M9 | RAB3GAP2 | Rab3 GTPase-activating |
| | NO: 160) | | | | protein non-catalytic |
| | | | | | subunit |
| | KLKEAVEAI (SEQ ID | A02 | Q13620 | CUL4B | Cullin-4B |
| | NO: 161) | | | | |
| | VLREAVEAV (SEQ ID | A02 | Q8IVB5 | LIX1L | LIX1-like protein |
| | NO: 162) | | | | |
| | LLDEAIQAV (SEQ ID | C16 | Q96QK1 | VP535 | Vacuolar protein |
| | NO: 163) | | | | sorting-associated |
| | | | | | protein 35 |
| | AMQEAIDAI (SEQ ID | A02 | 075037 | KIF21B | Kinesin-like protein |
| | NO: 164) | | | | KIF21B |
| | AADEALNAM (SEQ ID | C16 | Q13586 | STIM1 | Stromal interaction |
| | NO: 165) | | | | molecule 1 |
| Rizvi_60 | SSPLSHGSK (SEQ ID | A11 | | | |
| | NO: 166) | | | | |
| | HFDLSHGSA (SEQ ID | C16 | P69905 | HBA1 | Hemoglobin subunit |
| | NO: 167) | | | | alpha |
| Rizvi_64 | YVPTISHPI (SEQ ID | A02 | | | |
| | NO: 168) | | | | |
| | HSGTISQPR (SEQ ID | A11 | Q14667 | KIAA0100 | Protein KIAA0100 |
| | NO: 169) | | | | |
| Rizvi_65 | ALSKLVIRR (SEQ ID | A11 | | | |
| | NO: 170) | | | | |
| | SRMKLVLRW (SEQ ID | C07 | Q9H0X9 | OSBPL5 | Oxysterol-binding |
| | NO: 171) | | | | protein-related protein |
| | | | | | 5 |
| | RALKLIIRL (SEQ ID | C16 | O95197 | RTN3 | Reticulon-3 |
| | NO: 172) | | | | |
| | DYDKLIVRF (SEQ ID | B18 | P23381 | WARS | Tryptophan--tRNA |
| | NO: 173) | | | | ligase, cytoplasmic |
| | LLDKLLIRL (SEQ ID | A02 | O14646 | CHD1 | Chromodomain- |
| | NO: 174) | | | | helicase-DNA- |
| | | | | | binding protein 1 |
| Rizvi_66 | KRTALSKLV (SEQ ID | C07 | | | |
| | NO: 175) | | | | |
| | FPEALARLL (SEQ ID | B51 | O00329 | PIK3CD | Phosphatidylinositol |
| | NO: 176) | | | | 4,5-bisphosphate 3- |
| | | | | | kinase catalytic |
| | | | | | subunit delta isoform |
| | VAAALARLL (SEQ ID | C07 | Q8TCT7 | SPPL2B | Signal peptide |
| | NO: 177) | | | | peptidase-like 2B |
| | VAAALARLL (SEQ ID | C16 | Q8TCT7 | SPPL2B | Signal peptide |
| | NO: 177) | | | | peptidase-like 2B |
| Rizvi_68 | RHHESEPSL (SEQ ID | C07 | | | |
| | NO: 178) | | | | |
| | SAVESQPSR (SEQ ID | A11 | Q9Y520 | PRRC2C | Protein PRRC2C |
| | NO: 179) | | | | |
| | RHHESDPSL (SEQ ID | C07 | Q9C0K0 | BCL11B | B-cell |
| | NO: 180) | | | | lymphoma/leukemia |
| | | | | | 11B |
| Rizvi_71 | HLSPMAAEA (SEQ ID | A02 | | | |
| | NO: 181) | | | | |
| | HAAPMAAER (SEQ ID | A11 | P10588 | NR2F6 | Nuclear receptor |
| | NO: 182) | | | | subfamily 2 group F |
| | | | | | member 6 |
| Rizvi_73 | KEVKTSSTF (SEQ ID | B18 | | | |
| | NO: 183) | | | | |
| | QIFKTSATK (SEQ ID | A11 | P40616 | ARL1 | ADP-ribosylation |
| | NO: 184) | | | | factor-like protein 1 |
| | RPIKTATTL (SEQ ID | B51 | Q96KC8 | DNAJC1 | DnaJ homolog |
| | NO: 185) | | | | subfamily C member |
| | | | | | 1 |
| | FYIKTSTTV (SEQ ID | C07 | P29373 | CRABP2 | Cellular retinoic |
| | NO: 186) | | | | acid-binding protein 2 |
| Rizvi_77 | SISENQSLL (SEQ ID | C16 | | | |
| | NO: 187) | | | | |
| | NPSENRSLL (SEQ ID | B51 | Q4VCS5 | AMOT | Angiomotin |
| | NO: 188) | | | | |
| Rizvi_79 | LVFPLVMGV (SEQ ID | A02 | | | |
| | NO: 189) | | | | |
| | IPHPLIIVIGV (SEQ ID | B51 | P61201 | COPS2 | COP9 signalosome |
| | NO: 190) | | | | complex subunit 2 |
| Rizvi_82 | GVLVDSSHK (SEQ ID | A11 | | | |
| | NO: 191) | | | | |
| | IGYVDTTHW (SEQ ID | C16 | Q6UWU4 | C6orf89 | Bombesin receptor- |
| | NO: 192) | | | | activated protein |
| | | | | | C6oth39 |
| Rizvi_83 | YQSSSSTSV (SEQ ID | A02 | | | |
| | NO: 193) | | | | |
| | SPGSSSTSL (SEQ ID | B51 | Q99550 | MPHOSPH9 | M-phase |
| | NO: 194) | | | | phosphoprotein 9 |
| | YPTSSSTSF (SEQ ID | B18 | P50402 | EMD | Emerin |
| | NO: 195) | | | | |
| | YPTSSSTSF (SEQ ID | B51 | P50402 | EMD | Emerin |
| | NO: 195) | | | | |
| | ATHSSSTSW (SEQ ID | C16 | Q9UPN3 | MACF1 | Microtubule-actin |
| | NO: 196) | | | | cross-linking factor 1, |
| | | | | | isoforms 1/2/3/5 |
| Rizvi_85 | TLTEKLVAI (SEQ ID | A02 | | | |
| | NO: 197) | | | | |
| | EAIEKLVAL (SEQ ID | B51 | Q15257 | PTPA | Serine/threonine- |
| | NO: 198) | | | | protein phosphatase |
| | | | | | 2A activator |
| | QLQEKLVAL (SEQ ID | A02 | Q86UU1 | PHLDB1 | Pleckstrin homology-like |
| | NO: 199) | | | | domain family B |
| | | | | | member 1 |
| | TAMEKLVAR (SEQ ID | A11 | Q6PFW1 | PPIP5K1 | Inositol |
| | NO: 200) | | | | hexakisphosphate and |
| | | | | | diphosphoinositol- |
| | | | | | pentakisphosphate |
| | | | | | kinase 1 |
| Rizvi_93 | QLDGSSSSV (SEQ ID | A02 | | | |
| | NO: 201) | | | | |
| | RSYGSTASV (SEQ ID | C07 | Q8IV50 | LYSMD2 | LysM and putative |
| | NO: 202) | | | | peptidoglycan- |
| | | | | | binding domain- |
| | | | | | containing protein 2 |
| | YASGSSASL (SEQ ID | B51 | Q15149 | PLEC | Plectin |
| | NO: 203) | | | | |
| | YASGSSASL (SEQ ID | C07 | Q15149 | PLEC | Plectin |
| | NO: 203) | | | | |
| | KTIGSSASV (SEQ ID | A02 | O60870 | KIN | DNA/RNA-binding |
| | NO: 204) | | | | protein KIN17 |
| | AELGSSTSL (SEQ ID | B18 | O60232 | SSSCA1 | Sjoegren |
| | NO: 205) | | | | syndrome/scleroderm |
| | | | | | a autoantigen 1 |
| | TEVGSSSSA (SEQ ID | B18 | Q9ULT8 | HECTD1 | E3 ubiquitin-protein |
| | NO: 206) | | | | ligase HECTD1 |
| | NPAGSSSSL (SEQ ID | B18 | O15391 | YY2 | Transcription factor |
| | NO: 207) | | | | YY2 |
| | GSMGSTTSV (SEQ ID | A02 | Q14669 | TRIP12 | E3 ubiquitin-protein |
| | NO: 208) | | | | ligase TRIP12 |
| | LSHGSTTSY (SEQ ID | C07 | Q92539 | LPIN2 | Phosphatidate |
| | NO: 209) | | | | phosphatase LPIN2 |
| Rizvi_108 | TTHKKIHTV (SEQ ID | C16 | | | |
| | NO: 210) | | | | |
| | VLEKKFHTV (SEQ ID | A02 | Q99729 | HNRNPAB | Heterogeneous |
| | NO: 211) | | | | nuclear |
| | | | | | ribonucleoprotein |
| | | | | | A/B, isoform 2 |
| | SMKKKLHTL (SEQ ID | C16 | Q96Q15 | SMG1 | Serine/threonine- |
| | NO: 212) | | | | protein kinase SMG1 |
| | AEAKKIHTL (SEQ ID | B18 | Q9H4I2 | ZHX3 | Zinc fingers and |
| | NO: 213) | | | | homeoboxes protein 3 |
| | TEHKKIHTA (SEQ ID | B18 | Q9UII5 | ZNF107 | Zinc finger protein |
| | NO: 214) | | | | 107 |
| | NRHKKIHTV (SEQ ID | C07 | Q8N119 | ZNF664 | Zinc finger protein |
| | NO: 215) | | | | 664 |
| Rizvi_111 | LVKALLLYY (SEQ ID | A11 | | | |
| | NO: 216) | | | | |
| | LINALVLYV (SEQ ID | B51 | A5YKK6 | CNOT1 | CCR4-NOT |
| | NO: 217) | | | | transcription complex |
| | | | | | subunit 1 |
| | LINALVLYV (SEQ ID | C16 | A5YKK6 | CNOT1 | CCR4-NOT |
| | NO: 217) | | | | transcription complex |
| | | | | | subunit 1 |
| Rizvi_115 | MDFELEIEF (SEQ ID | B18 | | | |
| | NO: 218) | | | | |
| | ARHELQVEM (SEQ ID | C07 | O60610 | DIAPH1 | Protein diaphanous |
| | NO: 219) | | | | homolog 1 |
| | RLAELELEL (SEQ ID | A024 | Q9Y2E | DIP2C | Disco-interacting |
| | NO: 220) | | | | protein 2 homolog C |
| | RLAELELEL (SEQ ID | C16 | Q9Y2E4 | DIP2C | Disco-interacting |
| | NO: 220) | | | | protein 2 homolog C |
| Rizvi_116 | FELEIEFES (SEQ ID | B18 | | | |
| | NO: 221) | | | | |
| | RLVEIQYEL (SEQ ID | C16 | Q14161 | GIT2 | ARF GTPase- |
| | NO: 222) | | | | activating protein |
| | | | | | GIT2 |
| Rizvi_124 | IRNKTSGVV (SEQ ID | C07 | | | |
| | NO: 223) | | | | |
| | KAVKTTGVL (SEQ ID | C16 | Q9BXN2 | CLEC7A | C-type lectin domain |
| | NO: 224) | | | | family 7 member A |
| Rizvi_128 | KVIVVTPKV (SEQ ID | A02 | | | |
| | NO: 225) | | | | |
| | SSIVVSPKM (SEQ ID | C07 | Q9NRD1 | FBOXO6 | F-box only protein 6 |
| | NO: 226) | | | | |
| | SSIVVSPKM (SEQ ID | C16 | Q9NRD1 | FBOXO6 | F-box only protein 6 |
| | NO: 226) | | | | |
| Rizvi_129 | SGMFRNGLK (SEQ ID | A11 | | | |
| | NO: 227) | | | | |
| | GRNFRNPLA (SEQ ID | C07 | P06733 | ENOA | Alpha-enolase |
| | NO: 228) | | | | |
| Rizvi_131 | WVLVVVVGV (SEQ ID | A02 | | | |
| | NO: 229) | | | | |
| | QARVVVLGL (SEQ ID | C16 | Q15102 | PAFAH1B3 | Platelet-activating |
| | NO: 230) | | | | factor acetylhydrolase |
| | | | | | IB subunit gamma |
| | FPSVVLVGL (SEQ ID | B18 | P28838 | LAP3 | Cytosol |
| | NO: 231) | | | | aminopeptidase |
| Rizvi_142 | AAMSASSER (SEQ ID | A11 | | | |
| | NO: 232) | | | | |
| | MHSSAATEL (SEQ ID | C07 | Q2KHR3 | QSER1 | Glutamine and serine- |
| | NO: 233) | | | | rich protein 1 |
| | SPQSAAAEL (SEQ ID | B51 | Q12948 | FOXC1 | Forkhead box protein |
| | NO: 234) | | | | C1 |
| | LAASASAEF (SEQ ID | B51 | Q00325 | SLC25A3 | Phosphate carrier |
| | NO: 235) | | | | protein, mitochondrial |
| Rizvi_143 | FMIGTIIAK (SEQ ID | A11 | | | |
| | NO: 236) | | | | |
| | AEVGTIFAL (SEQ ID | B18 | Q96BZ9 | TBC1D20 | TBC1 domain family |
| | NO: 237) | | | | member 20 |
| | GRTGTFIAL (SEQ ID | C07 | P23469 | PTPRE | Receptor-type |
| | NO: 238) | | | | tyrosine-protein |
| | | | | | phosphatase epsilon |
| | KLLGTVVAL (SEQ ID | A02 | H7BY58 | PCMT1 | Protein-L-isoaspartate |
| | NO: 239) | | | | O-methyltransferase |
| | KLLGTVVAL (SEQ ID | C16 | H7BY58 | PCMT1 | Protein-L-isoaspartate |
| | NO: 239) | | | | O-methyltransferase |
| | HPSGTVVAI (SEQ ID | B58 | Q9HC35 | EML4 | Echinoderm |
| | NO: 240) | | | | microtubule-associated |
| | | | | | protein-like 4 |
| Rizvi_147 | ELLPLTPVL (SEQ ID | A02 | | | |
| | NO: 241) | | | | |
| | YTIPLSPVL (SEQ ID | A02 | Q9NPI6 | DCP1A | mRNA-decapping |
| | NO: 242) | | | | enzyme 1A |
| | YTIPLSPVL (SEQ ID | B51 | Q9NPI6 | DCP1A | mRNA-decapping |
| | NO: 242) | | | | enzyme 1A |
| | ALSPLSPVA (SEQ ID | A02 | Q96K8 | ZNF5213 | Zinc finger protein |
| | NO: 243) | | | | 521 |
| Rizvi_150 | ALGQAITLL (SEQ ID | A02 | | | |
| | NO: 244) | | | | |
| | DHSQAVTLI (SEQ ID | C07 | Q8WXH0 | SYNE2 | Nesprin-2 |
| | NO: 245) | | | | |
| Rizvi_151 | GMSPEVTLA (SEQ ID | A02 | | | |
| | NO: 246) | | | | |
| | ESLPEISLL (SEQ ID | B51 | Q6NUN7 | JHY | Jhy protein homolog |
| | NO: 247) | | | | |
| | ESLPEISLL (SEQ ID | C16 | Q6NUN7 | JHY | Jhy protein homolog |
| | NO: 247) | | | | |
| Rizvi_152 | VIFSAIHFL (SEQ ID | A02 | | | |
| | NO: 248) | | | | |
| | QYASAFHFL (SEQ ID | C07 | Q96RK4 | BBS4 | Bardet-Biedl |
| | NO: 249) | | | | syndrome 4 protein |
| Rizvi_153 | SAIHFLASL (SEQ ID | C16 | | | |
| | NO: 250) | | | | |
| | ILWHFVASL (SEQ ID | A02 | O75592 | MYCBP2 | E3 ubiquitin-protein |
| | NO: 251) | | | | ligase MYCBP2 |
| Rizvi_154 | FLASLALST (SEQ ID | A02 | | | |
| | NO: 252) | | | | |
| | RTHSLAVSL (SEQ ID | C07 | Q9NVX7 | KBTB4 | Kelch repeat and BTB |
| | NO: 253) | | | | domain-containing |
| | | | | | protein 4 |
| | SPDSLAVSL (SEQ ID | B51 | P06312 | IGKV4-1 | Immunoglobulin |
| | NO: 254) | | | | kappa vanable 4-1 |
| | TSVSLAVSR (SEQ ID | A11 | O94973 | AP2A2 | AP-2 complex subunit |
| | NO: 255) | | | | alpha-2 |
| Rizvi_155 | IHFLASLAL (SEQ ID | C07 | | | |
| | NO: 256) | | | | |
| | TAVLATIAF (SEQ ID | C16 | Q8TCT6 | SPPL3 | Signal peptide |
| | NO: 257) | | | | peptidase-like 3 |
| | RVTLATIAW (SEQ ID | C16 | P48060 | GLIPR1 | Glioma pathogenesis- |
| | NO: 258) | | | | related protein 1, |
| | | | | | isoform 2 |
| | TQALASVAY (SEQ ID | B18 | Q9P2A4 | ABI3 | ABI gene family |
| | NO: 259) | | | | member 3 |
| | TQSLASVAY (SEQ ID | B18 | Q8IZP0 | ABI1 | AbI interactor 1 |
| | NO: 260) | | | | |
| Rizvi_157 | VVAASAAAK (SEQ ID | A11 | | | |
| | NO: 261) | | | | |
| | DAPASAAAV (SEQ ID | B51 | O43488 | AKR7A2 | Aflatoxin B1 |
| | NO: 262) | | | | aldehyde reductase |
| | | | | | member 2 |
| | ALAASAAAV (SEQ ID | A02 | P26599 | PTBP1 | Polypyrimidinetract- |
| | NO: 263) | | | | binding protein 1 |
| | ATNASAAAF (SEQ ID | C16 | Q9NR56 | MBNL1 | Muscleblind-like |
| | NO: 264) | | | | protein 1, soform 5 |
| | IPAASAAAM (SEQ ID | B51 | Q9UQ35 | SRRM2 | Serine/arginine |
| | NO: 265) | | | | repetitive matrix |
| | | | | | protein 2 |
| Rizvi_159 | ALDANETLL (SEQ ID | A02 | | | |
| | NO: 266) | | | | |
| | LVSANQTLK (SEQ ID | A03 | Q86UV5 | U5P48 | Ubiquitin carboxyl- |
| | NO: 267) | | | | terminal hydrolase 48 |
| Rizvi_160 | NETLLLTGS (SEQ ID | B18 | | | |
| | NO: 268) | | | | |
| | KSHLLVTGF (SEQ ID | C07 | Q15269 | PWP2 | Periodic tryptophan |
| | NO: 269) | | | | protein 2 homolog |
| Rizvi_163 | KSHLLVTGF (SEQ ID | C16 | Q15269 | PWP2 | Periodic tryptophan |
| | NO: 269) | | | | protein 2 homolog |
| | RHTAHISEL (SEQ ID | C07 | | | |
| | NO: 270) | | | | |
| | TIMAHVTEF (SEQ ID | C07 | Q9Y4E5 | ZNF451 | E3 SUMO-protein |
| | NO: 271) | | | | ligase ZNF451 |
| | TIMAHVTEF (SEQ ID | C16 | Q9YLIE5 | ZNF451 | E3 SUMO-protein |
| | NO: 271) | | | | ligase ZNF451 |
| Rizvi_165 | GMFPVDKPV (SEQ ID | A02 | | | |
| | NO: 272) | | | | |
| | SESPVERPL (SEQ ID | B18 | Q96SB4 | SRPK1 | SRSF protein kinase 1 |
| | NO: 273) | | | | |
| | SQAPVNKPK (SEQ ID | A11 | Q15059 | BRD3 | Bromodomain- |
| | NO: 274) | | | | containing protein 3 |
| Rizvi_173 | FIQDISVKM (SEQ ID | C16 | | | |
| | NO: 275) | | | | |
| | LRFDISLKK (SEQ ID | C07 | Q8TCT9 | HM13 | Minor |
| | NO: 276) | | | | histocompatibility |
| | | | | | antigen H13 |
| | HLTDITLKV (SEQ ID | A02 | Q15046 | KARS | Lysine--tRNA ligase |
| | NO: 277) | | | | |
| | VPIDITVKL (SEQ ID | B51 | Q9Y5Q9 | GTF3C3 | General transcription |
| | NO: 278) | | | | factor 3C polypeptide |
| | | | | | 3 |
| | | | | | NADH |
| | FQLDITVKM (SEQ ID | A02 | P565566 | NDUFA | dehydrogenase |
| | NO: 279) | | | | [ubiquinone] 1 alpha |
| | | | | | sub complex subunit 6 |
| | | | | | NADH |
| | FQLDITVKM (SEQ ID | B18 | P565566 | NDUFA | dehydrogenase |
| | NO: 279) | | | | [ubiquinone] 1 alpha |
| | | | | | sub complex subunit 6 |
| | | | | | NADH |
| | FQLDITVKM (SEQ ID | C16 | P565566 | NDUFA | dehydrogenase |
| | NO: 279) | | | | [ubiquinone] 1 alpha |
| | | | | | sub complex subunit 6 |
| | RRGDITIKL (SEQ ID | C07 | Q8WWY8 | LIPH | Lipase member H |
| | NO: 280) | | | | |
| | EHLDIAIKL (SEQ ID | C07 | Q96LZ7 | RMDN2 | Regulator of |
| | NO: 281) | | | | microtubule dynamics |
| | | | | | protein 2, isoform 2 |
| | REHDIAIKF (SEQ ID | B18 | P30260 | CDC27 | Cell division cycle |
| | NO: 282) | | | | protein 27 homolog |
| Rizvi_175 | IHLHSSQVL (SEQ ID | C07 | | | |
| | NO: 283) | | | | |
| | KYIHSANVL (SEQ ID | C07 | Q16659 | MAPK6 | Mitogen-activated |
| | NO: 284) | | | | protein kinase 6 |
| | KYIHSANVL (SEQ ID | C16 | Q16659 | MAPK6 | Mitogen-activated |
| | NO: 284) | | | | protein kinase 6 |
| Rizvi_177 | FLHEIFHQV (SEQ ID | A02 | | | |
| | NO: 285) | | | | |
| | FISEIIHQL (SEQ ID | A02 | Q9C040 | TRIM2 | Tripartite motif- |
| | NO: 286) | | | | containing protein 2 |
| | FISEIIHQL (SEQ ID | C16 | Q9C040 | TRIM2 | Tripartite motif- |
| | NO: 286) | | | | containing protein 2 |
| Rizvi_182 | GSNINKSLK (SEQ ID | A11 | | | |
| | NO: 287) | | | | |
| | TRDINKALY (SEQ ID | C07 | O75891 | ALDH1L1 | Cytosolic 10- |
| | NO: 288) | | | | formyltetrahydrofolate |
| | | | | | dehydrogenase |
| Rizvi_184 | ESFSIYVYK (SEQ ID | A11 | | | |
| | NO: 289) | | | | |
| | ESYSIYVYK (SEQ ID | A11 | P06899 | HIST1H2BJ | |
| | NO: 290) | | | | Histone H2B type 1-J |
| Rizvi_186 | KQSASAVHV (SEQ ID | A02 | | | |
| | NO: 291) | | | | |
| | FNTASALHL (SEQ ID | C07 | Q06413 | MEF2C | Myocyte-specific |
| | NO: 292) | | | | enhancer factor 2C |
| | FNTASALHL (SEQ ID | C16 | Q06413 | MEF2C | Myocyte-specific |
| | NO: 292) | | | | enhancer factor 2C |
| | ASAASALHL (SEQ ID | C07 | Q6P2E9 | EDC4 | Enhancer of mRNA- |
| | NO: 293) | | | | decapping protein 4 |
| | ASAASALHL (SEQ ID | C16 | Q6P2E9 | EDC4 | Enhancer of mRNA- |
| | NO: 293) | | | | decapping protein 4 |
| Rizvi_187 | VHVPVSVAM (SEQ ID | C07 | | | |
| | NO: 294) | | | | |
| | TGSPVSIAL (SEQ ID | C16 | P57723 | PCBP4 | Poly(rC)-binding |
| | NO: 295) | | | | protein 4 |
| Rizvi_189 | KMLRIVELY (SEQ ID | A11 | | | |
| | NO: 296) | | | | |
| | YSLRIIDLI (SEQ ID | B51 | P50748 | KNTC1 | Kinetochore- |
| | NO: 297) | | | | associated protein 1 |
| Rizvi_196 | GRIELYRVV (SEQ ID | C07 | | | |
| | NO: 298) | | | | |
| | FMAELYRVL (SEQ ID | A02 | Q96FC9 | DDX11 | ATP-dependent DNA |
| | NO: 299) | | | | helicase DDX11 |
| | FMAELYRVL (SEQ ID | C16 | Q96FC9 | DDX11 | ATP-dependent DNA |
| | NO: 299) | | | | helicase DDX11 |
| | SPEELYRVF (SEQ ID | B51 | O95433 | AHSA1 | Activator of 90 kDa |
| | NO: 300) | | | | heat shock protein |
| | | | | | ATPase homolog 1 |
| | HRVELYKVL (SEQ ID | C07 | Q8N2K0 | ABHD12 | Monoacylglycerol |
| | NO: 301) | | | | lipase ABHD12 |
| Rizvi_197 | RIFSSSYVA (SEQ ID | A02 | | | |
| | NO: 302) | | | | |
| | VLLSSSFVY (SEQ ID | A11 | Q96PP9 | GBP4 | Guanylate-binding |
| | NO: 303) | | | | protein 4 |
| | VLLSSSFVY (SEQ ID | B18 | Q96PP9 | GBP4 | Guanylate-binding |
| | NO: 303) | | | | protein 4 |
| | VLLSSSFVY (SEQ ID | C16 | Q96PP9 | GBP4 | Guanylate-binding |
| | NO: 303) | | | | protein 4 |
| Rizvi_199 | SSYVAFISY (SEQ ID | A11 | | | |
| | NO: 304) | | | | |
| | GRIVAFFSF (SEQ ID | C07 | Q07817 | BCL2L1 | Bc1-2-like protein 1 |
| | NO: 305) | | | | |
| Rizvi_202 | HIIPFQPQK (SEQ ID | A11 | | | |
| | NO: 306) | | | | |
| | KLLPFNPQL (SEQ ID | A02 | O94919 | ENDOD1 | Endonuclease |
| | NO: 307) | | | | domain-containing 1 |
| | | | | | protein |
| | KLLPFNPQL (SEQ ID | C16 | O94919 | ENDOD1 | Endonuclease |
| | NO: 307) | | | | domain-containing 1 |
| | | | | | protein |
| Rizvi_203 | LRRTTDRKL (SEQ ID | C07 | | | |
| | NO: 308) | | | | |
| | LRKTTEKKL (SEQ ID | C07 | Q7LGA3 | HS2ST1 | Heparan sulfate 2-0- |
| | NO: 309) | | | | sulfotransferase 1 |
| Rizvi_208 | TNTDHLFTV (SEQ ID | C16 | | | |
| | NO: 310) | | | | |
| | FLFDHLLTL (SEQ ID | B18 | Q7L2H7 | EIF3M | Eukaryotic translation |
| | NO: 311) | | | | initiation factor 3 |
| | | | | | subunit M |
| | ALLDHLITH (SEQ ID | A11 | Q8IVC4 | ZNF584 | Zinc finger protein |
| | NO: 312) | | | | 584 |
| Rizvi_209 | GLLGVWTVL (SEQ ID | A02 | | | |
| | NO: 313) | | | | |
| | TPAGVYTVF (SEQ ID | B51 | O15417 | TNRC18 | Trinucleotide repeat- |
| | NO: 314) | | | | containing gene 18 |
| | | | | | protein |
| Rizvi_210 | LLGVWTVLL (SEQ ID | A02 | | | |
| | NO: 315) | | | | |
| | METVWTILP (SEQ ID | B18 | P00403 | MT-CO2 | Cytochromec oxidase |
| | NO: 316) | | | | subunit 2 |
| Rizvi_211 | GVWTVLLLL (SEQ ID | A02 | | | |
| | NO: 317) | | | | |
| | SAITVFLLF (SEQ ID | B18 | O75352 | MPDU1 | Mannose-P-dolichol |
| | NO: 318) | | | | utilization defect 1 |
| | | | | | protein |
| | SAITVFLLF (SEQ ID | B51 | O75352 | MPDU1 | Mannose-P-dolichol |
| | NO: 318) | | | | utilization defect 1 |
| | | | | | protein |
| | APRTVLLLL (SEQ ID | B51 | P30480 | HLA-B | HLA class I |
| | NO: 319) | | | | histocompatibility |
| | | | | | antigen, B-42 alpha |
| | | | | | chain |
| Rizvi_212 | LHNVGLLGV (SEQ ID | C07 | | | |
| | NO: 320) | | | | |
| | | | | | HLA class II |
| | GLTVGLVGI (SEQ ID | A02 | P01903 | HLA- | histocompatibility |
| | NO: 321) | | | DRA | antigen, DR alpha |
| | | | | | chain |
| | AVKVGLVGR (SEQ ID | A11 | P58107 | EPPK1 | Epiplakin |
| | NO: 322) | | | | |
| Rizvi_213 | GLLGSWTVL (SEQ ID | A02 | | | |
| | NO: 323) | | | | |
| | SAGGSFTVR (SEQ ID | A11 | P08238 | HSP90AB1 | Heat shock protein |
| | NO: 324) | | | | HSP 90-beta |
| | HMDGSFSVK (SEQ ID | A11 | O60291 | MGRN1 | E3 ubiquitin-protein |
| | NO: 325) | | | | ligase MGRN1 |
| Rizvi_218 | YIALLFGAK (SEQ ID | A11 | | | |
| | NO: 326) | | | | |
| | APSLLYGAL (SEQ ID | B51 | Q96L91 | EP400 | E1A-binding protein |
| | NO: 327) | | | | p400 |
| | KQQLLIGAY (SEQ ID | B18 | Q99832 | CCT7 | T-complex protein 1 |
| | NO: 328) | | | | subunit eta |
| Rizvi_223 | SVGQDLLLY (SEQ ID | A11 | | | |
| | NO: 329) | | | | |
| | KLNQDVLLV (SEQ ID | A02 | Q9UPZ3 | HPS5 | Hermansky-Pudlak |
| | NO: 330) | | | | syndrome 5 protein |
| | KLNQDVLLV (SEQ ID | C16 | Q9UPZ3 | HPS5 | Hermansky-Pudlak |
| | NO: 330) | | | | syndrome 5 protein |
| Rizvi_224 | SLFSELSPV (SEQ ID | A02 | | | |
| | NO: 331) | | | | |
| | STASELSPK (SEQ ID | A11 | Q3KQU3 | MAP7D1 | MAP7 domain-containing |
| | NO: 332) | | | | protein 1 |
| Rizvi_225 | TVAPVSVPR (SEQ ID | A11 | | | |
| | NO: 333) | | | | |
| | VVGPVSLPR (SEQ ID | A11 | Q12802 | AKAP13 | A-kinase anchor |
| | NO: 334) | | | | protein 13 |

*If no suballele is indicated like B4402, then HLA allele is a 01 subalele, e.g. A25 is A25:01, or A02 is A02:01, etc.
⁺If blank, then sequence refers to a neoepitope.

TABLE 3
Neoepitope and HLA-LM

| | | presenting | | | |
|---|---|---|---|---|---|
| neoepitope | | HLA | uniprot | Gene | Protein name of |
| identifier | Sequence | allele* | identifier⁺ | name⁺ | HLA-LM⁺ |
| Ott_6 | TELERFLEY | B4402 | | | |
| | (SEQ ID NO: 8) | | | | |
| | QLIERILEA | A02 | Q5H9R7 | PPP6R3 | Serine/threonine |
| | (SEQ ID NO: 9) | | | | -protein |
| | | | | | phosphatase 6 |
| | | | | | regulatory |
| | | | | | subunit 3 |
| Ott_7 | LLHTELERF | B15 | | | |
| | (SEQ ID NO: 10) | | | | |
| | YLRTELERL | A02 | Q5VUA4 | ZNF318 | Zinc finger |
| | (SEQ ID NO: 11) | | | | protein 318 |
| Ott_8 | TLFHTFYEL | A02 | | | |
| | (SEQ ID NO: 12) | | | | |
| | VYHHTFFEM | A24 | P49588 | AARS | Alanine--tRNA |
| | (SEQ ID NO: 13) | | | | ligase, |
| | | | | | cytoplasmic |
| | SLLHTIYEV | A02 | Q969G9 | NKD1 | Protein naked |
| | (SEQ ID NO: 14) | | | | cuticle homolog |
| | | | | | 1 |
| | SLMHTIYEV | A02 | Q969F2 | NKD2 | Protein naked |
| | (SEQ ID NO: 15) | | | | cuticle homolog |
| | | | | | 2 |
| Ott_11 | KLFESKAEL | A02 | | | |
| | (SEQ ID NO: 16) | | | | |
| | RVYESKAEF | B15 | O75153 | CLUB | Clustered |
| | (SEQ ID NO: 17) | | | | mitochondria |
| | | | | | protein homolog |
| | QEAESKSEL | B4402 | Q9BZH6 | WDR11 | WD repeat- |
| | (SEQ ID NO: 18) | | | | containing |
| | | | | | protein 11 |
| | AEAESRAEA | B4402 | Q14764 | MVP | Maj or vault |
| | (SEQ ID NO: 19) | | | | protein |
| Ott_13 | GIPENSFNV | | A02 | | |
| | (SEQ ID NO: 20) | | | | |
| | RLPENTFNI | A24 | Q8IVU3 | HERC6 | Probable E3 |
| | (SEQ ID NO: 21) | | | | ubiquitin- |
| | | | | | protein ligase |
| | | | | | HERC6 |
| Ott_25 | NVLSSLVLV | A02 | | | |
| | (SEQ ID NO: 22) | | | | |
| | HLLSSLLLY | A03 | Q07002 | CDK18 | Cyclin- |
| | (SEQ ID NO: 23) | | | | dependent |
| | | | | | kinase 18 |
| | TGFSSLFLK | A03 | Q8N201 | INTS1 | Integrator |
| | (SEQ ID NO: 24) | | | | complex subunit |
| | | | | | 1 |
| Ott_26 | RLMLRKVAL | A02 | | | |
| | (SEQ ID NO: 25) | | | | |
| | TESLRKIAL | B47 | Q96NL6 | SCLT1 | Sodium channel |
| | (SEQ ID NO: 26) | | | | and clathrin |
| | | | | | linker 1 |
| Ott_27 | ALQSQSISL | A02 | | | |
| | (SEQ ID NO: 27) | | | | |
| | SQCSQSLSV | B47 | Q9NVI1 | FANCI | Fanconi anemia |
| | (SEQ ID NO: 28) | | | | group I protein |
| Ott_31 | KLNFRLFVI | A02 | | | |
| | (SEQ ID NO: 29) | | | | |
| | SRLFRVFVH | B2705 | Q96BX8 | MOB3A | MOB kinase |
| | (SEQ ID NO: 30) | | | | activator 3A |
| Ott_32 | FEAEFTQVA | B18 | | | |
| | (SEQ ID NO: 31) | | | | |
| | FAAEFSNVM | A25 | Q9UDY8 | MALT1 | Mucosa- |
| | (SEQ ID NO: 32) | | | | associated |
| | | | | | lymphoid tissue |
| | | | | | lymphoma |
| | | | | | translocation |
| | | | | | protein 1 |
| Ott_38 | WLVDLLPST | A02 | | | |
| | (SEQ ID NO: 33) | | | | |
| | SVDDLLPSL | A02 | Q14289 | PTK2B | Protein-tyrosine |
| | (SEQ ID NO: 34) | | | | kinase 2-beta |
| | DLIDLVPSL | A25 | P47756-2 | CAPZB | F-actin-capping |
| | (SEQ ID NO: 35) | | | | protein subunit |
| | beta | | | | |
| | SRIDLIPSL | B2702 | Q99567 | Nup88 | Nuclear pore |
| | (SEQ ID NO: 36) | | | | complex protein |
| | Nup88 | | | | |

**If no suballele is indicated like B4402, all HLA alleles are 01 suballeles, e.g. A25 is A25:01, or A02 is A02:01, etc.
⁺If blank, then sequence refers to a neoepitope.

TABLE 4
Neoepitope and HLA-LM

| | | presenting | | | |
|---|---|---|---|---|---|
| neoepitope | | HLA | uniprot | Gene | Protein name |
| identifier | Sequence | allele* | identifier⁺ | name⁺ | of HLA-LM⁺ |
| Ott_66 | TSIQSPSLY | A01 | | | |
| | (SEQ ID NO: 45) | | | | |
| | RTAQSGALR | A66 | P40222 | TXLNA | Alpha-taxilin |
| | (SEQ ID NO: 46) | | | | |
| Ott_67 | HLARHRHLM | B08 | | | |
| | (SEQ ID NO: 47) | | | | |
| | FVFRHKQLL | B08 | Q9NYV6 | RRN3 | RNA |
| | (SEQ ID NO: 48) | | | | polymerase |
| | | | | | I-specific |
| | | | | | transcription |
| | | | | | initiation |
| | | | | | factor RRN3 |
| Ott_70 | HTLGAASSF | A66 | | | |
| | (SEQ ID NO: 49) | | | | |
| | GSDGAASSY | A01 | Q14203 | DCTN1 | Dynactin |
| | (SEQ ID NO: 50) | | | | subunit 1 |
| Ott_73 | NVELRRNVL | B08 | | | |
| | (SEQ ID NO: 51) | | | | |
| | NPDLRRNVL | B08 | Q15560 | TCEA2 | Transcription |
| | (SEQ ID NO: 52) | | | | elongation |
| | | | | | factor A |
| | | | | | protein 2 |
| | NPNLRKNVL | B08 | P23193 | TCEA1 | Transcription |
| | (SEQ ID NO: 53) | | | | elongation |
| | | | | | factor A |
| | | | | | protein 1 |
| Ott_75 | SIKEITNFK | A66 | | | |
| | (SEQ ID NO: 54) | | | | |
| | TVAEISQFL | A66 | Q9Y689 | ARL5A | ADP- |
| | (SEQ ID NO: 55) | | | | ribosylation |
| | | | | | factor-like |
| | | | | | protein 5A |
| Ott_76 | ESIKEITNF | A66 | | | |
| | (SEQ ID NO: 56) | | | | |
| | DVRKEVTNV | A66 | Q15746 | MYLK | Myosin light |
| | (SEQ ID NO: 57) | | | | chain kinase, |
| | | | | | smooth muscle |

*If no suballele is indicated like B4402, all HLA alleles are 01 suballeles, e.g. A25 is A25:01, or A02 is A02:01, etc.
⁺If blank, then sequence refers to a neoepitope.

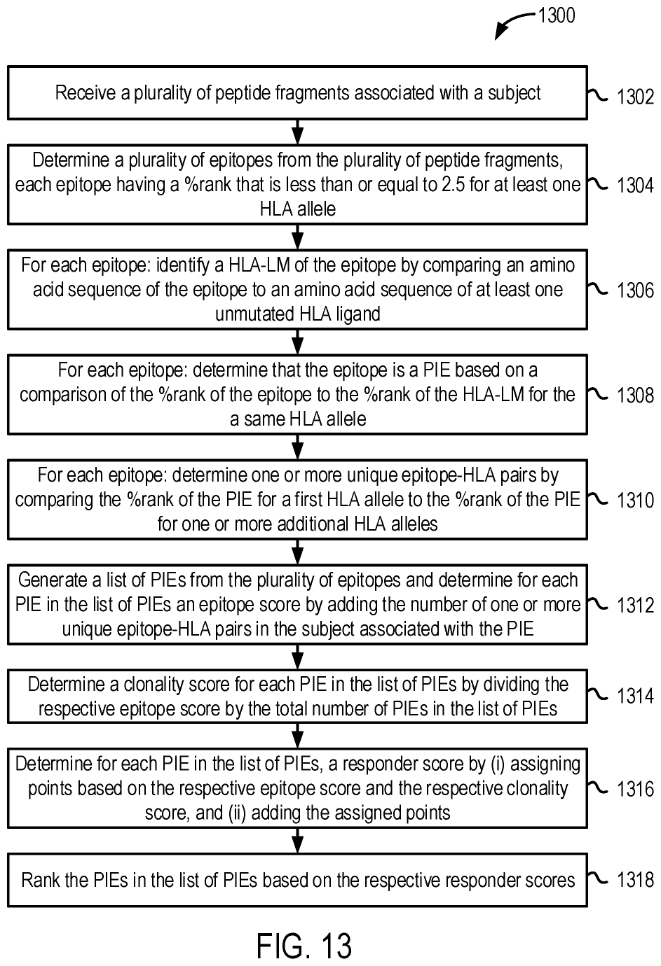

FIG. 13 shows a flow diagram of an example process 1300 for determining the efficacy of a therapeutic regimen in a subject. In particular, the process 1300 determines the efficacy of epitopes to generate an immune response in the subject. The process 1300 can be executed, for example, by the epitope data processing system 120 shown in FIG. 1C. The process 1300 includes receiving a plurality of peptide fragments associated with a subject (1302). At least one example of this process stage has been discussed above. In particular, as discussed in relation to FIGS. 2A-2C, the complete neoepitope dataset can be derived from a set of peptide fragments received from a peptide sequencing device. As an example, the peptide sequencing device may include one or more of mass spectrometry based sequencers or Edman degradation based sequencers. The peptide fragments can be associated with a single subject or a set of subjects. The epitope data processing system 120 may receive a data file including the sequences of each of the peptide fragments sequenced by the sequencer.

The process 1300 further includes determining a plurality of epitopes from the plurality of peptide fragments, each epitope having a % rank that is less than or equal to 2.5 for at least one HLA allele (1304). At least one example of this process stage is discussed above. In particular, as discussed above, the plurality of peptide fragments can be considered a epitopes if their affinity (% rank) for binding to at least one HLA allele is equal to or above the threshold value of 2.5. The epitope data processing system 120 can determine the % rank of each of the plurality of peptide fragments, and then determine the plurality of epitopes based on those epitopes that have an associated % rank that is greater than or equal to 2.5.

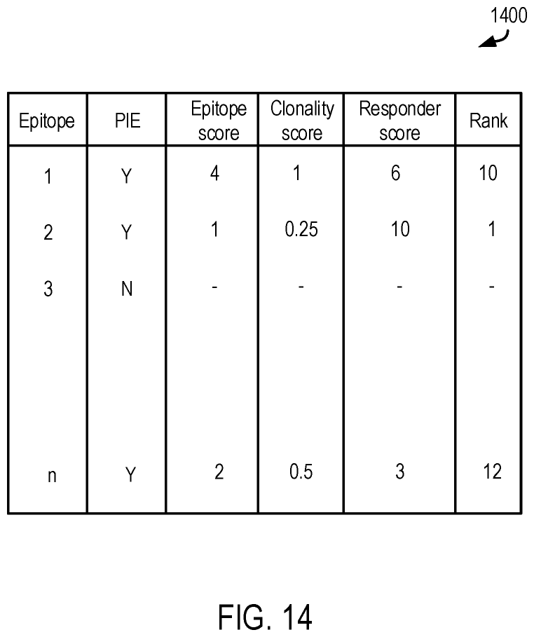

FIG. 14 shows an epitope data structure 1400 for storing information regarding the epitopes. In particular, the epitope data processing system 120 can store the epitope data structure 1400 in memory, and update the data structure 1400 based on the data processing discussed herein. For example, the epitope data processing system 120 can list the plurality of epitopes determined above into the “Epitope” column of the data structure 1400.

The process 1300 further includes, for each epitope in the plurality of epitopes, identifying, a HLA-LM of the epitope by comparing an amino acid sequence of the epitope to an amino acid sequence of at least one unmutated HLA ligand, wherein the HLA-LM binds to the at least one HLA allele (1306). At least one example of this process stage has been discussed above (e.g., section: “Identifying a human leukocyte antigen ligand match (HLA-LM)”). As an example, the epitope data processing system 120 can identify an HLA-LM by comparing the amino acid sequence of the epitope to the amino acid sequence of 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, or 1000 or more HLA ligands. In some embodiments, identifying an HLA-LM comprises comparing the amino acid sequence of the epitope to the amino acid sequence of at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 or more HLA ligands. In some embodiments, identifying an HLA-LM comprises comparing the amino acid sequence of the epitope to the amino acid sequence of at least 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, or 1000 or more HLA ligands.

The process 1300 further includes, for each epitope in the plurality of epitopes, determining that the epitope is a potentially immunogenic epitope (PIE) based on a comparison of the % rank of the epitope to the % rank of the HLA-LM for the same HLA allele (1308). At least one example of this process stage is discussed above (e.g., section: “characterizing an epitope as a potentially immunogenic epitope (PIE)”). The epitope data processing system 120 can base the determination of whether the epitope is a PIE on a comparison of the affinities of the epitope and the HLA-LM with the same HLA allele. In particular, the epitope data processing system 120 can compare the % rank of the epitope with the % rank of the HLA-LM with respect to the same HLA allele. The epitope data processing system 120 can update the epitope data structure 1400 to indicate which ones of the epitopes listed are PIE. For example, the epitope data processing system 120 can indicate “Y” against the epitope determined to be a PIE, and a “N” against the epitope determined not to be a PIE.

The process 1300 further includes determining one or more unique epitope-HLA pairs by comparing the % rank of the PIE for a first HLA allele to the % rank of the PIE for one or more additional HLA alleles (1310). At least one example of this process stage is discussed above (e.g., “Unique epitope-HLA pairs, clonality score, epitope score, responder score” and FIGS. 10A-10C). The epitope data processing system 120 can determine unique epitope-HLA pairs by determining that the % rank of the PIE for one HLA allele is within a certain range of that of the PIE for other HLA alleles. The range can be a factor (e.g., multiples) of the % rank of the PLE for the one HLA allele.

The process 1300 further includes generating a list of PIEs from the plurality of epitopes, the list of PIEs including epitopes from the plurality of epitopes that have been determined as a PIE (1312). At least one example of this process stage is discussed above (e.g., “Unique epitope-HLA pairs, clonality score, epitope score, responder score”). The epitope data processing system can generate a list of PIEs from the epitopes that are determined to be PIEs. As an example, the epitope data processing system 120 can list the PIE in the data structure 1400 shown in FIG. 14. The list of PIEs can include those epitopes that have a “Y” in the PIE column of the data structure 1300.

The process 1300 further includes determining for each PIE in the list of PIEs an epitope score by adding the number of one or more unique epitope-HLA pairs in the subject associated with the PIE (1312). At least one example of this process stage is discussed above (e.g., “Unique epitope-HLA pairs, clonality score, epitope score, responder score,” and FIGS. 10A-10C). The epitope data processing system 120, in some examples, determine the epitope score based on the number of unique epitope-HLA pairs. The epitope data processing system 120 can update the data structure 1400 by including the epitope score in the epitope column for each epitope identified as a PIE. For example, as shown in FIG. 14, the data structure 1400 includes an epitope score of 4 for epitope “1” an epitope score of 1 for epitope “2”, no epitope score for epitope “3”, as this epitope is not a PIE, and an epitope score of “2” for the nth epitope.

The process 1300 further includes determining a clonality score for each PIE in the list of PIEs by dividing the respective epitope score by the total number of PIEs in the list of PIEs (1314). At least one example of this process stage is discussed above (e.g., “Unique epitope-HLA pairs, clonality score, epitope score, responder score,” and FIGS. 10A-10C). The epitope data processing system 120 can determine clonality scores for each PIE. For example, the epitope data processing system 120 can determine the clonality score by dividing the epitope score by the total number of PIEs in the list of PIEs, as shown in the examples of FIGS. 10A-10C. The epitope data processing system 120 can update the data structure 1400 with the clonality score corresponding with each of the PIEs. For example, as shown in FIG. 14, the epitope data processing system 120 can update the “clonality score” column of the data structure 1400 with clonality scores of “1”, “0.25”, and “0.5” corresponding to epitopes “1”, “2”, and “n” respectively.

The process 1300 further includes determining for each PIE in the list of PIEs, a responder score by (i) assigning points based on the respective epitope score and the respective clonality score, and (ii) adding the assigned points (1316). At least one example of this process stage is discussed above (e.g., sections: “Unique epitope-HLA pairs, clonality score, epitope score, responder score,” “Prediction of response to immune checkpoint blockade via RESPONDER score,” and FIGS. 10A-10C). The epitope data processing system 120 can determine a responder score for each PIE. As an example, the responder score can be based on assigned points corresponding to the clonality score and the epitope scores of a PIE. The epitope data processing system 120 can then add the points associated with clonality score and the epitope score to determine the responder score. The epitope data processing system 120 can update the data structure 1400 with the responder score associated with each of the epitopes identified as PIEs.

The process 1300 further includes ranking the PIEs in the list of PIEs based on the respective responder scores (1318). As shown in FIG. 14, the epitope data processing system 120 can update the data structure 1400 with a rank associated with each PIE based on the responder score. For example, the epitope data processing system 120 can assign a rank proportional to the responder score. For example, the epitope data processing system 120 can assign a highest rank “1” to the epitope having the highest responder score, and assign progressively lower ranks to epitopes with progressively lower responder scores. The ranks can indicate the efficacy of that epitopes in generating an immune response in a subject. The epitope data processing system 120 can display the ranking of each of the PIEs on a display device for viewing. The rankings can then be utilized to select the appropriate epitope for a therapeutic regiment.

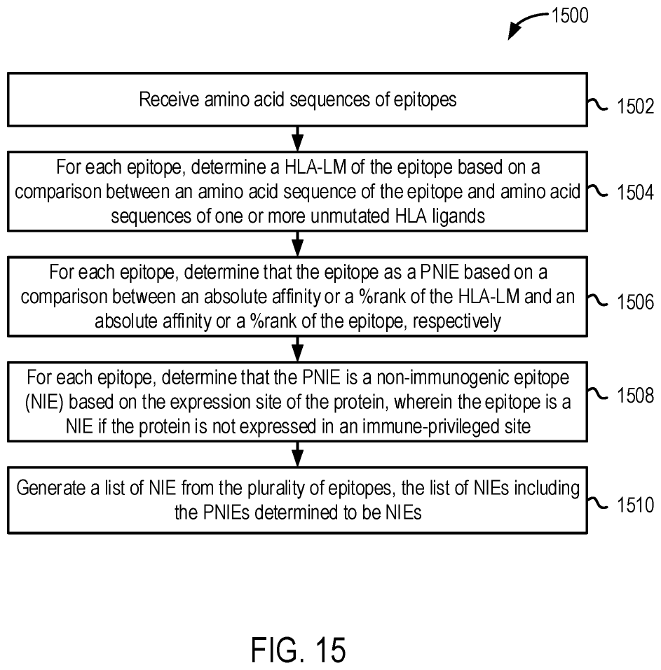

FIG. 15 shows a flow diagram of an example process 1500 for determining an immunogenicity of an epitope derived from a protein. The process 1500 can be executed, for example, by the epitope data processing system 120 discussed above in relation to FIG. 1C. The process 1500 includes receiving amino acid sequences associated with a plurality of epitopes (1502). At least one example of this process stage is discussed above. In particular, as discussed in relation to FIG. 2A, the complete neoepitope dataset can be received from a peptide sequencing device. As an example, the peptide sequencing device may include one or more of mass spectrometry based sequencers or Edman degradation based sequencers. The neoepitope dataset can include amino acid sequences associated with each of the epitopes included in the dataset. The epitope data processing system 120 may receive a data file including the amino acid sequences of each of plurality of epitopes sequenced by the sequencer.

The process 1500 further includes for each epitope, determining from a database, a HLA-LM of the epitope based on a comparison between an amino acid sequence of the epitope and amino acid sequences of one or more unmutated human leukocyte antigen HLA ligands (1504). At least one example of this process stage is discussed above (e.g., section: “Identifying a human leukocyte antigen ligand match (HLA-LM)”). As an example, the epitope data processing system 120 can identify an HLA-LM by comparing the amino acid sequence of the epitope to the amino acid sequence of 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, or 1000 or more HLA ligands. In some embodiments, identifying an HLA-LM comprises comparing the amino acid sequence of the epitope to the amino acid sequence of at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 or more HLA ligands. In some embodiments, identifying an HLA-LM comprises comparing the amino acid sequence of the epitope to the amino acid sequence of at least 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, or 1000 or more HLA ligands.

The process 1500 further includes, for each epitope, determining, by the one or more processors, that the epitope as a potentially non-immunogenic epitope (PNIE) based on a comparison between an absolute affinity or a % rank of the HLA-LM and an absolute affinity or a % rank of the epitope, respectively (1506). At least example of this process stage is discussed above. (e.g., section: “Characterizing an epitope as a potentially non-immunogenic epitope (PNIE)”). The absolute affinity of the HLA-LM can be a binding affinity of the HLA-LM to a human leukocyte antigen (HLA) allele and the absolute affinity of the epitope can be a predicted binding affinity of the epitope to the HLA allele. The % rank of the HLA-LM can be an absolute affinity at which the HLA-LM binds to an HLA allele relative to an absolute affinity at which one or more peptides bind to the HLA allele. The % rank of the epitope can be an absolute affinity at which the epitope binds to the HLA allele relative to an absolute affinity at which one or more peptides bind to the HLA. For example, the epitope data processing system 120 can determine an epitope as a PNIE when the absolute affinity of the HLA-LM for an HLA is within a 3, 4, 5, 6, 7, 8, 9, or 10-fold range of the absolute affinity of the epitope for the same HLA.

The process 1500 further includes determining that the PNIE is a non-immunogenic epitope (NIE) based on the expression site of the protein, wherein the epitope is a NIE if the protein is not expressed in an immune-privileged site (1508). At least one example of this process stage is discussed above (e.g., “Characterizing an epitope as a non-immunogenic epitope (NIE)”). In some examples, the epitope data processing system 120 can determine the immune-privileged site to be a site in the body that is able to tolerate the introduction of antigens without eliciting an inflammatory immune response. In some embodiments, an immune-privileged site is selected from an eye, placenta, fetus, testicle, central nervous system, and hair follicle. In some embodiments, the hair follicle is an anagen hair follicle.

The process 1500 further includes generating a list of NIEs from the plurality of epitopes, the list of NIEs including the PNIEs determined to be NIEs (1510). The epitope data processing system can generate a list of NIEs from the PNIEs where the NIEs do not include the epitopes that are expressed in immune privileged sites. As a result, the epitope data processing system 120 generates a list that includes a subset of previously identified epitopes that are likely to generate an immune response in the subject. Thus, the list of NIEs can be improve the effectiveness of therapeutic regimens that include epitopes.

1. A computer implemented method of determining the efficacy of a therapeutic regimen in a subject in need thereof, the method comprising:
receiving, by one or more processors, from a peptide sequencing device, a plurality of peptide fragments associated with the subject;
determining, by the one or more processors, a plurality of epitopes from the plurality of peptide fragments, each epitope of the plurality of epitopes having a % rank that is less than or equal to 2.5 for at least one human leukocyte antigen (HLA) allele;
for each epitope of the plurality of epitopes:
identifying, by the one or more processors, a human leukocyte antigen ligand match (HLA-LM) of the epitope by comparing an amino acid sequence of the epitope to an amino acid sequence of at least one unmutated human leukocyte antigen (HLA) ligand, wherein the HLA-LM binds to at least one HLA allele,
determining, by the one or more processors, that the epitope is a potentially immunogenic epitope (PIE) based on a comparison of the % rank of the epitope to the % rank of the HLA-LM for the same HLA allele, and
determining, by the one or more processors, one or more unique epitope-HLA pairs by comparing the % rank of the PIE for a first HLA allele to the % rank of the PIE for one or more additional HLA alleles, optionally wherein for each of the one or more unique epitope-HLA pairs, the % rank of the respective PIE for the first HLA allele is not within a 5-fold range of the % rank of the respective PIE for the respective one or more additional HLA alleles;
generating, by the one or more processors, a list of PIEs from the plurality of epitopes, the list of PIEs including epitopes from the plurality of epitopes that have been determined as a PIE;
determining, by the one or more processors, for each PIE in the list of PIEs an epitope score by adding the number of one or more unique epitope-HLA pairs associated with the PIE;
determining, by the one or more processors, a clonality score for each PIE in the list of PIEs by dividing the respective epitope score by the total number of PIEs in the list of PIEs;
determining, by the one or more processors, for each PIE in the list of PIEs, a responder score by (i) assigning points based on the respective epitope score and the respective clonality score, and (ii) adding the assigned points; and
ranking, by the one or more processors, the PIEs in the list of PIEs based on the respective responder scores, optionally wherein comparing the amino acid sequence of the epitope to the amino acid sequence of one or more unmutated HLA ligands comprises performing a sequence alignment of the amino acid sequences.

2. (canceled)

3. The computer implemented method of claim 1, further comprising determining, by the one or more processors, a match score for a T cell receptor (TCR) recognition area that is located within the sequence alignment, optionally wherein the TCR recognition area comprises a region of 5 amino acids.

4. (canceled)

5. The computer implemented method of claim 3, wherein determining the match score comprises assigning, by the one or more processors, a numerical value to one or more amino acid positions within the TCR recognition area, wherein assigning a numerical value is based on the similarity of the amino acid residues at the one or more amino acid positions within the TCR recognition area, optionally wherein
a numerical value of 1 is assigned to an amino acid position within the TCR recognition area based on the amino acid residue of the epitope and the amino acid residue of the at least one unmutated HLA ligand at said amino acid position being identical, or
the numerical value assigned to an amino acid position within the TCR recognition area is based on the values provided in FIG. 5,
the match score is the sum of the numerical values assigned to the one or more amino acid positions within the TCR recognition area, or
the HLA ligand is identified as an HLA-LM based on the match score being greater than or equal to 4, or
the HLA ligand is identified as an HLA-LM based on amino acid residues at two, three, or more amino acid positions of the respective epitope being identical to amino acid residues at corresponding positions of the HLA ligand, optionally wherein the identical amino acid residues are located at one or both ends of the TCR recognition area.

6. (canceled)

7. (canceled)

8. (canceled)

9. (canceled)

10. (canceled)

11. (canceled)

12. (canceled)

13. The computer implemented method of claim 1, wherein the epitope is not classified as a PIE based on the respective HLA-LM having a % rank of less than or equal to 4 for at least one HLA allele or wherein the epitope is not classified as a PIE based on the % rank of the respective HLA-LM being within a 5-fold range of the % rank of the epitope.

14. (canceled)

15. (canceled)

16. The computer implemented method of claim 1, wherein 6 points are assigned when the epitope score is greater than 200, 4 points are assigned when the epitope score is greater than 50 and less than or equal to 200, or 2 points are assigned when the epitope score is less than or equal to 50; or
wherein 3 points are assigned when the clonality score is greater than 0.7 and less than or equal to 0.84, 2 points are assigned when the clonality score is less than or equal to 0.7, or 1 point is assigned when the clonality score is greater than 0.84.

17. (canceled)

18. The computer implemented method of claim 1, wherein the therapeutic regimen is effective when the responder score is greater than or equal to 7, or wherein the therapeutic regimen is not effective when the responder score is less than or equal to 6.

19. (canceled)

20. The computer implemented method of claim 18, further comprising indicating, by the one or more processors, a modification recommendation to the therapeutic regimen and/or administration of one or more additional therapies upon determining that the therapeutic regimen is not effective, optionally wherein indicating the modification recommendation to the therapeutic regimen comprises indicating a recommendation for increasing the dose and/or dosing frequency of the therapeutic regimen, or indicating a recommendation of terminating the therapeutic regimen.

21. (canceled)

22. (canceled)

23. The computer implemented method of claim 1, wherein each epitope of the plurality of epitopes is derived from a protein selected from a cancer-specific protein, a viral protein, a bacterial protein, a parasitic protein, and a fungal protein and/or wherein the subject is suffering from cancer or an infection, optionally wherein
the cancer is selected from the group consisting of melanoma, non-small cell lung cancer (NSCLC), cutaneous squamous skin carcinoma, small cell lung cancer (SCLC), hormone-refractory prostate cancer, triple-negative breast cancer, microsatellite instable tumor, renal cell carcinoma, urothelial carcinoma, Hodgkin's lymphoma, and Merkel cell carcinoma, or
the infection is selected from the group consisting of a viral infection, bacterial infection, parasitic infection, and fungal infection.

24. (canceled)

25. (canceled)

26. (canceled)

27. The computer implemented method of claim 1, wherein the therapeutic regimen is selected from among an anti-cancer therapy, an anti-viral therapy, an anti-bacterial therapy, an anti-parasitic therapy, and an anti-fungal therapy, optionally wherein the anti-cancer therapy is an immune checkpoint blockade therapy selected from an anti-PD1 therapy, an anti-PDL1 therapy, and an anti-CTLA4 therapy.

28. (canceled)

29. (canceled)

30. A computer implemented method for determining the immunogenicity of an epitope derived from a protein, the method comprising:
receiving, by one or more processors, amino acid sequences associated with a plurality of epitopes;
for each epitope of the plurality of epitopes:
determining, by the one or more processors, from a database, a human leukocyte antigen ligand match (HLA-LM) of the epitope based on a comparison between an amino acid sequence of the epitope and amino acid sequences of one or more unmutated human leukocyte antigen (HLA) ligands;
determining, by the one or more processors, that the epitope as a potentially non-immunogenic epitope (PNIE) based on a comparison between an absolute affinity or a % rank of the HLA-LM and an absolute affinity or a % rank of the epitope, respectively, wherein:
the absolute affinity of the HLA-LM is a binding affinity of the HLA-LM to a human leukocyte antigen (HLA) allele and the absolute affinity of the epitope is a predicted binding affinity of the epitope to the HLA allele, and/or
the % rank of the HLA-LM is an absolute affinity at which the HLA-LM binds to an HLA allele relative to an absolute affinity at which one or more peptides bind to the HLA allele, and the % rank of the epitope is an absolute affinity at which the epitope binds to the HLA allele relative to an absolute affinity at which one or more peptides bind to the HLA allele; and
determining, by the one or more processors, that the PNIE is a non-immunogenic epitope (NIE) based on the expression site of the protein, wherein the epitope is a NIE if the protein is not expressed in an immune-privileged site, optionally wherein the immune-privileged site is selected from the group consisting of eye, placenta, fetus, testicle, central nervous system, and hair follicle; and
generating, by the one or more processors, a list of NIEs from the plurality of epitopes, the list of NIEs including the PNIEs determined to be NIEs, optionally wherein comparing the amino acid sequence of the epitope to the amino acid sequence of one or more HLA ligands comprises performing, by the one or more processors, a sequence alignment of the amino acid sequences.

31. (canceled)

32. The computer implemented method of claim 30, further comprising determining, by the one or more processors, a match score for a T cell receptor (TCR) recognition area that is located within the sequence alignment, optionally wherein the TCR recognition area comprises a region of 5 amino acids.

33. (canceled)

34. The computer implemented method of claim 32, wherein determining the match score comprises assigning, by the one or more processors, a numerical value to one or more amino acid positions within the TCR recognition area, wherein assigning a numerical value is based on the similarity of the amino acid residues at the one or more amino acid positions within the TCR recognition area optionally wherein
a numerical value of 1 is assigned to an amino acid position within the TCR recognition area based on the amino acid residue of the epitope and the amino acid residue of the at least one unmutated HLA ligand at said amino acid position being identical, or
the numerical value assigned to an amino acid position within the TCR recognition area is based on the values provided in FIG. 5,
the match score is the sum of the numerical values assigned to the one or more amino acid positions within the TCR recognition area, or
the at least one unmutated HLA ligand is identified as an HLA-LM based on the match score being greater than or equal to 4, or
the at least one unmutated HLA ligand is identified as an HLA-LM based on amino acid residues at two, three, or more amino acid positions of the epitope being identical to amino acid residues at corresponding positions of the at least one unmutated HLA ligand, optionally wherein the identical amino acid residues are located at one or both ends of the TCR recognition area.

35. (canceled)

36. (canceled)

37. (canceled)

38. (canceled)

39. (canceled)

40. (canceled)

41. (canceled)

42. The computer implemented method of claim 30, wherein the epitope is characterized as a PNIE based on the HLA-LM having a % rank of less than or equal to 4 for at least one HLA allele or wherein the epitope is characterized as a PNIE based on the absolute affinity or % rank of the HLA-LM being within a 5-fold range of the absolute affinity or % rank of the epitope, respectively.

43. (canceled)

44. (canceled)

45. A composition comprising a vector that includes a polynucleotide encoding an epitope listed in any of Tables 2-4, optionally wherein the vector is a bacterial plasmid and/or further comprises a eukaryotic promoter, or the polynucleotide comprises deoxyribonucleic acid (DNA).

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)


SEQUENCE LISTING (346 sequences)

SEQ ID NO: 1 - protein, 9 aa, Homo sapiens
KIWEELSML

SEQ ID NO: 2 - protein, 9 aa, Homo sapiens
EVDPIGHVY

SEQ ID NO: 3 - protein, 9 aa, Homo sapiens
SAAAVFSHF

SEQ ID NO: 4 - protein, 9 aa, Homo sapiens
KVVAVNDPF

SEQ ID NO: 5 - protein, 9 aa, Homo sapiens
TLGTVILLV

SEQ ID NO: 6 - protein, 9 aa, Homo sapiens
ETSKQVTRW

SEQ ID NO: 7 - protein, 9 aa, Homo sapiens
SLKKQLTRV

SEQ ID NO: 8 - protein, 9 aa, Homo sapiens
TELERFLEY

SEQ ID NO: 9 - protein, 9 aa, Homo sapiens
QLIERILEA

SEQ ID NO: 10 - protein, 9 aa, Homo sapiens
LLHTELERF

SEQ ID NO: 11 - protein, 9 aa, Homo sapiens
YLRTELERL

SEQ ID NO: 12 - protein, 9 aa, Homo sapiens
TLFHTFYEL

SEQ ID NO: 13 - protein, 9 aa, Homo sapiens
VYHHTFFEM

SEQ ID NO: 14 - protein, 9 aa, Homo sapiens
SLLHTIYEV

SEQ ID NO: 15 - protein, 9 aa, Homo sapiens
SLMHTIYEV

SEQ ID NO: 16 - protein, 9 aa, Homo sapiens
KLFESKAEL

SEQ ID NO: 17 - protein, 9 aa, Homo sapiens
RVYESKAEF

SEQ ID NO: 18 - protein, 9 aa, Homo sapiens
QEAESKSEL

SEQ ID NO: 19 - protein, 9 aa, Homo sapiens
AEAESRAEA

SEQ ID NO: 20 - protein, 9 aa, Homo sapiens
GIPENSFNV

SEQ ID NO: 21 - protein, 9 aa, Homo sapiens
RLPENTFNI

SEQ ID NO: 22 - protein, 9 aa, Homo sapiens
NVLSSLVLV

SEQ ID NO: 23 - protein, 9 aa, Homo sapiens
HLLSSLLLY

SEQ ID NO: 24 - protein, 9 aa, Homo sapiens
TGFSSLFLK

SEQ ID NO: 25 - protein, 9 aa, Homo sapiens
RLMLRKVAL

SEQ ID NO: 26 - protein, 9 aa, Homo sapiens
TESLRKIAL

SEQ ID NO: 27 - protein, 9 aa, Homo sapiens
ALQSQSISL

SEQ ID NO: 28 - protein, 9 aa, Homo sapiens
SQCSQSLSV

SEQ ID NO: 29 - protein, 9 aa, Homo sapiens
KLNFRLFVI

SEQ ID NO: 30 - protein, 9 aa, Homo sapiens
SRLFRVFVH

SEQ ID NO: 31 - protein, 9 aa, Homo sapiens
FEAEFTQVA

SEQ ID NO: 32 - protein, 9 aa, Homo sapiens
FAAEFSNVM

SEQ ID NO: 33 - protein, 9 aa, Homo sapiens
WLVDLLPST

SEQ ID NO: 34 - protein, 9 aa, Homo sapiens
SVDDLLPSL

SEQ ID NO: 35 - protein, 9 aa, Homo sapiens
DLIDLVPSL

SEQ ID NO: 36 - protein, 9 aa, Homo sapiens
SRIDLIPSL

SEQ ID NO: 37 - protein, 9 aa, Homo sapiens
REFDKIELA

SEQ ID NO: 38 - protein, 9 aa, Homo sapiens
TAVDKVELF

SEQ ID NO: 39 - protein, 9 aa, Homo sapiens
AEVDKLELM

SEQ ID NO: 40 - protein, 9 aa, Homo sapiens
ALPQSILLF

SEQ ID NO: 41 - protein, 9 aa, Homo sapiens
RQDQSIILL

SEQ ID NO: 42 - protein, 9 aa, Homo sapiens
RVDQSLLLY

SEQ ID NO: 43 - protein, 9 aa, Homo sapiens
TIIDNIKEM

SEQ ID NO: 44 - protein, 9 aa, Homo sapiens
YGYDNVKEY

SEQ ID NO: 45 - protein, 9 aa, Homo sapiens
TSIQSPSLY

SEQ ID NO: 46 - protein, 9 aa, Homo sapiens
RTAQSGALR

SEQ ID NO: 47 - protein, 9 aa, Homo sapiens
HLARHRHLM

SEQ ID NO: 48 - protein, 9 aa, Homo sapiens
FVFRHKQLL

SEQ ID NO: 49 - protein, 9 aa, Homo sapiens
HTLGAASSF

SEQ ID NO: 50 - protein, 9 aa, Homo sapiens
GSDGAASSY

SEQ ID NO: 51 - protein, 9 aa, Homo sapiens
NVELRRNVL

SEQ ID NO: 52 - protein, 9 aa, Homo sapiens
NPDLRRNVL

SEQ ID NO: 53 - protein, 9 aa, Homo sapiens
NPNLRKNVL

SEQ ID NO: 54 - protein, 9 aa, Homo sapiens
SIKEITNFK

SEQ ID NO: 55 - protein, 9 aa, Homo sapiens
TVAEISQFL

SEQ ID NO: 56 - protein, 9 aa, Homo sapiens
ESIKEITNF

SEQ ID NO: 57 - protein, 9 aa, Homo sapiens
DVRKEVTNV

SEQ ID NO: 58 - protein, 9 aa, Homo sapiens
FMASNDEGV

SEQ ID NO: 59 - protein, 9 aa, Homo sapiens
KIISNEEGY

SEQ ID NO: 60 - protein, 9 aa, Homo sapiens
FLLLVAAMI

SEQ ID NO: 61 - protein, 9 aa, Homo sapiens
KLSLVAAML

SEQ ID NO: 62 - protein, 9 aa, Homo sapiens
FQDDDQTRL

SEQ ID NO: 63 - protein, 9 aa, Homo sapiens
FQDDDQTRV

SEQ ID NO: 64 - protein, 9 aa, Homo sapiens
KAIESFLEK

SEQ ID NO: 65 - protein, 9 aa, Homo sapiens
FTDESYLEL

SEQ ID NO: 66 - protein, 9 aa, Homo sapiens
SASESILEL

SEQ ID NO: 67 - protein, 9 aa, Homo sapiens
KLLMSQANV

SEQ ID NO: 68 - protein, 9 aa, Homo sapiens
KLVMSQANV

SEQ ID NO: 69 - protein, 9 aa, Homo sapiens
YTHNLIFVF

SEQ ID NO: 70 - protein, 9 aa, Homo sapiens
QLNNLVYVV

SEQ ID NO: 71 - protein, 9 aa, Homo sapiens
SHDNLVYVY

SEQ ID NO: 72 - protein, 9 aa, Homo sapiens
YTAQIILAL

SEQ ID NO: 73 - protein, 9 aa, Homo sapiens
KTSQIFLAK

SEQ ID NO: 74 - protein, 9 aa, Homo sapiens
FGDVGSTLF

SEQ ID NO: 75 - protein, 9 aa, Homo sapiens
TSDVGATLL

SEQ ID NO: 76 - protein, 9 aa, Homo sapiens
FLKELLVRI

SEQ ID NO: 77 - protein, 9 aa, Homo sapiens
TMLELLLRL

SEQ ID NO: 78 - protein, 9 aa, Homo sapiens
FPGELLLRL

SEQ ID NO: 79 - protein, 9 aa, Homo sapiens
ILAELLLRV

SEQ ID NO: 80 - protein, 9 aa, Homo sapiens
RELVHRILL

SEQ ID NO: 81 - protein, 9 aa, Homo sapiens
SDMVHRFLL

SEQ ID NO: 82 - protein, 9 aa, Homo sapiens
RPYVHKILV

SEQ ID NO: 83 - protein, 9 aa, Homo sapiens
YLVDSVAKM

SEQ ID NO: 84 - protein, 9 aa, Homo sapiens
YLVDSVAKT

SEQ ID NO: 85 - protein, 9 aa, Homo sapiens
SLFALGNVI

SEQ ID NO: 86 - protein, 9 aa, Homo sapiens
FHLALGQVL

SEQ ID NO: 87 - protein, 9 aa, Homo sapiens
FALGNVISA

SEQ ID NO: 88 - protein, 9 aa, Homo sapiens
MPFGNVISA

SEQ ID NO: 89 - protein, 9 aa, Homo sapiens
MPFGNVVSA

SEQ ID NO: 90 - protein, 9 aa, Homo sapiens
FLMASISSF

SEQ ID NO: 91 - protein, 9 aa, Homo sapiens
AVAASISSK

SEQ ID NO: 92 - protein, 9 aa, Homo sapiens
FLPASVASL

SEQ ID NO: 93 - protein, 9 aa, Homo sapiens
SAAASVASR

SEQ ID NO: 94 - protein, 9 aa, Homo sapiens
EIPASVSSY

SEQ ID NO: 95 - protein, 9 aa, Homo sapiens
TVPASFSSL

SEQ ID NO: 96 - protein, 9 aa, Homo sapiens
ISAASFSSL

SEQ ID NO: 97 - protein, 9 aa, Homo sapiens
AQFKGAWIL

SEQ ID NO: 98 - protein, 9 aa, Homo sapiens
FLPKGAYIY

SEQ ID NO: 99 - protein, 9 aa, Homo sapiens
LMASISSFL

SEQ ID NO: 100 - protein, 9 aa, Homo sapiens
GLTSISTFL

SEQ ID NO: 101 - protein, 9 aa, Homo sapiens
NQASITSFL

SEQ ID NO: 102 - protein, 9 aa, Homo sapiens
IMDSIAAFL

SEQ ID NO: 103 - protein, 9 aa, Homo sapiens
FQPSFSHLV

SEQ ID NO: 104 - protein, 9 aa, Homo sapiens
FAASFAHLL

SEQ ID NO: 105 - protein, 9 aa, Homo sapiens
FLQFRGNEV

SEQ ID NO: 106 - protein, 9 aa, Homo sapiens
LSSFRGQEF

SEQ ID NO: 107 - protein, 9 aa, Homo sapiens
VSSFRPNEF

SEQ ID NO: 108 - protein, 9 aa, Homo sapiens
GSLDVLMAV

SEQ ID NO: 109 - protein, 9 aa, Homo sapiens
SRLDVLLAL

SEQ ID NO: 110 - protein, 9 aa, Homo sapiens
FAADVLMAI

SEQ ID NO: 111 - protein, 9 aa, Homo sapiens
KITDVIMAF

SEQ ID NO: 112 - protein, 9 aa, Homo sapiens
VTYSGKFLI

SEQ ID NO: 113 - protein, 9 aa, Homo sapiens
LIYSGKLLL

SEQ ID NO: 114 - protein, 9 aa, Homo sapiens
FSKSGRLLL

SEQ ID NO: 115 - protein, 9 aa, Homo sapiens
GTWSGRVLV

SEQ ID NO: 116 - protein, 9 aa, Homo sapiens
VTGRLASGK

SEQ ID NO: 117 - protein, 9 aa, Homo sapiens
VVLRLATGF

SEQ ID NO: 118 - protein, 9 aa, Homo sapiens
TSDILKIPK

SEQ ID NO: 119 - protein, 9 aa, Homo sapiens
VPEILRVPL

SEQ ID NO: 120 - protein, 9 aa, Homo sapiens
KHLQVNITL

SEQ ID NO: 121 - protein, 9 aa, Homo sapiens
RQAQVNLTV

SEQ ID NO: 122 - protein, 9 aa, Homo sapiens
RLNQVNVTF

SEQ ID NO: 123 - protein, 9 aa, Homo sapiens
TKSSYTWFM

SEQ ID NO: 124 - protein, 9 aa, Homo sapiens
PAESYTFFI

SEQ ID NO: 125 - protein, 9 aa, Homo sapiens
RTLGQAFEV

SEQ ID NO: 126 - protein, 9 aa, Homo sapiens
STIGQAFEL

SEQ ID NO: 127 - protein, 9 aa, Homo sapiens
STWDSWNER

SEQ ID NO: 128 - protein, 9 aa, Homo sapiens
KAKDSFNEK

SEQ ID NO: 129 - protein, 9 aa, Homo sapiens
LESPALPMI

SEQ ID NO: 130 - protein, 9 aa, Homo sapiens
DFDPALGMM

SEQ ID NO: 131 - protein, 9 aa, Homo sapiens
NEAPLILPQ

SEQ ID NO: 132 - protein, 9 aa, Homo sapiens
SRVPLLLPL

SEQ ID NO: 133 - protein, 9 aa, Homo sapiens
LISPLLLPV

SEQ ID NO: 134 - protein, 9 aa, Homo sapiens
ELFPLIFPA

SEQ ID NO: 135 - protein, 9 aa, Homo sapiens
FNMSYKYPI

SEQ ID NO: 136 - protein, 9 aa, Homo sapiens
DAISYRFPR

SEQ ID NO: 137 - protein, 9 aa, Homo sapiens
GLQSFQMLV

SEQ ID NO: 138 - protein, 9 aa, Homo sapiens
LVNSFQLLY

SEQ ID NO: 139 - protein, 9 aa, Homo sapiens
SNHDLIQRL

SEQ ID NO: 140 - protein, 9 aa, Homo sapiens
KLNDLIQRL

SEQ ID NO: 141 - protein, 9 aa, Homo sapiens
MVKDLINRM

SEQ ID NO: 142 - protein, 9 aa, Homo sapiens
QTYDLIERR

SEQ ID NO: 143 - protein, 9 aa, Homo sapiens
AIYDLIERI

SEQ ID NO: 144 - protein, 9 aa, Homo sapiens
GEFDLVQRI

SEQ ID NO: 145 - protein, 9 aa, Homo sapiens
ASLETGFAK

SEQ ID NO: 146 - protein, 9 aa, Homo sapiens
ASVETGFAK

SEQ ID NO: 147 - protein, 9 aa, Homo sapiens
SLETGFAKK

SEQ ID NO: 148 - protein, 9 aa, Homo sapiens
LEHTGFSKA

SEQ ID NO: 149 - protein, 9 aa, Homo sapiens
LEAAGLLTY

SEQ ID NO: 150 - protein, 9 aa, Homo sapiens
ALWAGLLTL

SEQ ID NO: 151 - protein, 9 aa, Homo sapiens
KSYAGFLTV

SEQ ID NO: 152 - protein, 9 aa, Homo sapiens
LIVMFPFLL

SEQ ID NO: 153 - protein, 9 aa, Homo sapiens
MVKMFPLLV

SEQ ID NO: 154 - protein, 9 aa, Homo sapiens
VMFPFLLIL

SEQ ID NO: 155 - protein, 9 aa, Homo sapiens
ILIPFMLIL

SEQ ID NO: 156 - protein, 9 aa, Homo sapiens
IEHEHLNQY

SEQ ID NO: 157 - protein, 9 aa, Homo sapiens
LPVEHVNQL

SEQ ID NO: 158 - protein, 9 aa, Homo sapiens
RLQEAVEAA

SEQ ID NO: 159 - protein, 9 aa, Homo sapiens
SLQEAVQAA

SEQ ID NO: 160 - protein, 9 aa, Homo sapiens
HLIEAVEAI

SEQ ID NO: 161 - protein, 9 aa, Homo sapiens
KLKEAVEAI

SEQ ID NO: 162 - protein, 9 aa, Homo sapiens
VLREAVEAV

SEQ ID NO: 163 - protein, 9 aa, Homo sapiens
LLDEAIQAV

SEQ ID NO: 164 - protein, 9 aa, Homo sapiens
AMQEAIDAI

SEQ ID NO: 165 - protein, 9 aa, Homo sapiens
AADEALNAM

SEQ ID NO: 166 - protein, 9 aa, Homo sapiens
SSPLSHGSK

SEQ ID NO: 167 - protein, 9 aa, Homo sapiens
HFDLSHGSA

SEQ ID NO: 168 - protein, 9 aa, Homo sapiens
YVPTISHPI

SEQ ID NO: 169 - protein, 9 aa, Homo sapiens
HSGTISQPR

SEQ ID NO: 170 - protein, 9 aa, Homo sapiens
ALSKLVIRR

SEQ ID NO: 171 - protein, 9 aa, Homo sapiens
SRMKLVLRW

SEQ ID NO: 172 - protein, 9 aa, Homo sapiens
RALKLIIRL

SEQ ID NO: 173 - protein, 9 aa, Homo sapiens
DYDKLIVRF

SEQ ID NO: 174 - protein, 9 aa, Homo sapiens
LLDKLLIRL

SEQ ID NO: 175 - protein, 9 aa, Homo sapiens
KRTALSKLV

SEQ ID NO: 176 - protein, 9 aa, Homo sapiens
FPEALARLL

SEQ ID NO: 177 - protein, 9 aa, Homo sapiens
VAAALARLL

SEQ ID NO: 178 - protein, 9 aa, Homo sapiens
RHHESEPSL

SEQ ID NO: 179 - protein, 9 aa, Homo sapiens
SAVESQPSR

SEQ ID NO: 180 - protein, 9 aa, Homo sapiens
RHHESDPSL

SEQ ID NO: 181 - protein, 9 aa, Homo sapiens
HLSPMAAEA

SEQ ID NO: 182 - protein, 9 aa, Homo sapiens
HAAPMAAER

SEQ ID NO: 183 - protein, 9 aa, Homo sapiens
KEVKTSSTF

SEQ ID NO: 184 - protein, 9 aa, Homo sapiens
QIFKTSATK

SEQ ID NO: 185 - protein, 9 aa, Homo sapiens
RPIKTATTL

SEQ ID NO: 186 - protein, 9 aa, Homo sapiens
FYIKTSTTV

SEQ ID NO: 187 - protein, 9 aa, Homo sapiens
SISENQSLL

SEQ ID NO: 188 - protein, 9 aa, Homo sapiens
NPSENRSLL

SEQ ID NO: 189 - protein, 9 aa, Homo sapiens
LVFPLVMGV

SEQ ID NO: 190 - protein, 9 aa, Homo sapiens
IPHPLIMGV

SEQ ID NO: 191 - protein, 9 aa, Homo sapiens
GVLVDSSHK

SEQ ID NO: 192 - protein, 9 aa, Homo sapiens
IGYVDTTHW

SEQ ID NO: 193 - protein, 9 aa, Homo sapiens
YQSSSSTSV

SEQ ID NO: 194 - protein, 9 aa, Homo sapiens
SPGSSSTSL

SEQ ID NO: 195 - protein, 9 aa, Homo sapiens
YPTSSSTSF

SEQ ID NO: 196 - protein, 9 aa, Homo sapiens
ATHSSSTSW

SEQ ID NO: 197 - protein, 9 aa, Homo sapiens
TLTEKLVAI

SEQ ID NO: 198 - protein, 9 aa, Homo sapiens
EAIEKLVAL

SEQ ID NO: 199 - protein, 9 aa, Homo sapiens
QLQEKLVAL

SEQ ID NO: 200 - protein, 9 aa, Homo sapiens
TAMEKLVAR

SEQ ID NO: 201 - protein, 9 aa, Homo sapiens
QLDGSSSSV

SEQ ID NO: 202 - protein, 9 aa, Homo sapiens
RSYGSTASV

SEQ ID NO: 203 - protein, 9 aa, Homo sapiens
YASGSSASL

SEQ ID NO: 204 - protein, 9 aa, Homo sapiens
KTIGSSASV

SEQ ID NO: 205 - protein, 9 aa, Homo sapiens
AELGSSTSL

SEQ ID NO: 206 - protein, 9 aa, Homo sapiens
TEVGSSSSA

SEQ ID NO: 207 - protein, 9 aa, Homo sapiens
NPAGSSSSL

SEQ ID NO: 208 - protein, 9 aa, Homo sapiens
GSMGSTTSV

SEQ ID NO: 209 - protein, 9 aa, Homo sapiens
LSHGSTTSY

SEQ ID NO: 210 - protein, 9 aa, Homo sapiens
TTHKKIHTV

SEQ ID NO: 211 - protein, 9 aa, Homo sapiens
VLEKKFHTV

SEQ ID NO: 212 - protein, 9 aa, Homo sapiens
SMKKKLHTL

SEQ ID NO: 213 - protein, 9 aa, Homo sapiens
AEAKKIHTL

SEQ ID NO: 214 - protein, 9 aa, Homo sapiens
TEHKKIHTA

SEQ ID NO: 215 - protein, 9 aa, Homo sapiens
NRHKKIHTV

SEQ ID NO: 216 - protein, 9 aa, Homo sapiens
LVKALLLYY

SEQ ID NO: 217 - protein, 9 aa, Homo sapiens
LINALVLYV

SEQ ID NO: 218 - protein, 9 aa, Homo sapiens
MDFELEIEF

SEQ ID NO: 219 - protein, 9 aa, Homo sapiens
ARHELQVEM

SEQ ID NO: 220 - protein, 9 aa, Homo sapiens
RLAELELEL

SEQ ID NO: 221 - protein, 9 aa, Homo sapiens
FELEIEFES

SEQ ID NO: 222 - protein, 9 aa, Homo sapiens
RLVEIQYEL

SEQ ID NO: 223 - protein, 9 aa, Homo sapiens
IRNKTSGVV

SEQ ID NO: 224 - protein, 9 aa, Homo sapiens
KAVKTTGVL

SEQ ID NO: 225 - protein, 9 aa, Homo sapiens
KVIVVTPKV

SEQ ID NO: 226 - protein, 9 aa, Homo sapiens
SSIVVSPKM

SEQ ID NO: 227 - protein, 9 aa, Homo sapiens
SGMFRNGLK

SEQ ID NO: 228 - protein, 9 aa, Homo sapiens
GRNFRNPLA

SEQ ID NO: 229 - protein, 9 aa, Homo sapiens
WVLVVVVGV

SEQ ID NO: 230 - protein, 9 aa, Homo sapiens
QARVVVLGL

SEQ ID NO: 231 - protein, 9 aa, Homo sapiens
FPSVVLVGL

SEQ ID NO: 232 - protein, 9 aa, Homo sapiens
AAMSASSER

SEQ ID NO: 233 - protein, 9 aa, Homo sapiens
MHSSAATEL

SEQ ID NO: 234 - protein, 9 aa, Homo sapiens
SPQSAAAEL

SEQ ID NO: 235 - protein, 9 aa, Homo sapiens
LAASASAEF

SEQ ID NO: 236 - protein, 9 aa, Homo sapiens
FMIGTIIAK

SEQ ID NO: 237 - protein, 9 aa, Homo sapiens
AEVGTIFAL

SEQ ID NO: 238 - protein, 9 aa, Homo sapiens
GRTGTFIAL

SEQ ID NO: 239 - protein, 9 aa, Homo sapiens
KLLGTVVAL

SEQ ID NO: 240 - protein, 9 aa, Homo sapiens
HPSGTVVAI

SEQ ID NO: 241 - protein, 9 aa, Homo sapiens
ELLPLTPVL

SEQ ID NO: 242 - protein, 9 aa, Homo sapiens
YTIPLSPVL

SEQ ID NO: 243 - protein, 9 aa, Homo sapiens
ALSPLSPVA

SEQ ID NO: 244 - protein, 9 aa, Homo sapiens
ALGQAITLL

SEQ ID NO: 245 - protein, 9 aa, Homo sapiens
DHSQAVTLI

SEQ ID NO: 246 - protein, 9 aa, Homo sapiens
GMSPEVTLA

SEQ ID NO: 247 - protein, 9 aa, Homo sapiens
ESLPEISLL

SEQ ID NO: 248 - protein, 9 aa, Homo sapiens
VIFSAIHFL

SEQ ID NO: 249 - protein, 9 aa, Homo sapiens
QYASAFHFL

SEQ ID NO: 250 - protein, 9 aa, Homo sapiens
SAIHFLASL

SEQ ID NO: 251 - protein, 9 aa, Homo sapiens
ILWHFVASL

SEQ ID NO: 252 - protein, 9 aa, Homo sapiens
FLASLALST

SEQ ID NO: 253 - protein, 9 aa, Homo sapiens
RTHSLAVSL

SEQ ID NO: 254 - protein, 9 aa, Homo sapiens
SPDSLAVSL

SEQ ID NO: 255 - protein, 9 aa, Homo sapiens
TSVSLAVSR

SEQ ID NO: 256 - protein, 9 aa, Homo sapiens
IHFLASLAL

SEQ ID NO: 257 - protein, 9 aa, Homo sapiens
TAVLATIAF

SEQ ID NO: 258 - protein, 9 aa, Homo sapiens
RVTLATIAW

SEQ ID NO: 259 - protein, 9 aa, Homo sapiens
TQALASVAY

SEQ ID NO: 260 - protein, 9 aa, Homo sapiens
TQSLASVAY

SEQ ID NO: 261 - protein, 9 aa, Homo sapiens
VVAASAAAK

SEQ ID NO: 262 - protein, 9 aa, Homo sapiens
DAPASAAAV

SEQ ID NO: 263 - protein, 9 aa, Homo sapiens
ALAASAAAV

SEQ ID NO: 264 - protein, 9 aa, Homo sapiens
ATNASAAAF

SEQ ID NO: 265 - protein, 9 aa, Homo sapiens
IPAASAAAM

SEQ ID NO: 266 - protein, 9 aa, Homo sapiens
ALDANETLL

SEQ ID NO: 267 - protein, 9 aa, Homo sapiens
LVSANQTLK

SEQ ID NO: 268 - protein, 9 aa, Homo sapiens
NETLLLTGS

SEQ ID NO: 269 - protein, 9 aa, Homo sapiens
KSHLLVTGF

SEQ ID NO: 270 - protein, 9 aa, Homo sapiens
RHTAHISEL

SEQ ID NO: 271 - protein, 9 aa, Homo sapiens
TIMAHVTEF

SEQ ID NO: 272 - protein, 9 aa, Homo sapiens
GMFPVDKPV

SEQ ID NO: 273 - protein, 9 aa, Homo sapiens
SESPVERPL

SEQ ID NO: 274 - protein, 9 aa, Homo sapiens
SQAPVNKPK

SEQ ID NO: 275 - protein, 9 aa, Homo sapiens
FIQDISVKM

SEQ ID NO: 276 - protein, 9 aa, Homo sapiens
LRFDISLKK

SEQ ID NO: 277 - protein, 9 aa, Homo sapiens
HLTDITLKV

SEQ ID NO: 278 - protein, 9 aa, Homo sapiens
VPIDITVKL

SEQ ID NO: 279 - protein, 9 aa, Homo sapiens
FQLDITVKM

SEQ ID NO: 280 - protein, 9 aa, Homo sapiens
RRGDITIKL

SEQ ID NO: 281 - protein, 9 aa, Homo sapiens
EHLDIAIKL

SEQ ID NO: 282 - protein, 9 aa, Homo sapiens
REHDIAIKF

SEQ ID NO: 283 - protein, 9 aa, Homo sapiens
IHLHSSQVL

SEQ ID NO: 284 - protein, 9 aa, Homo sapiens
KYIHSANVL

SEQ ID NO: 285 - protein, 9 aa, Homo sapiens
FLHEIFHQV

SEQ ID NO: 286 - protein, 9 aa, Homo sapiens
FISEIIHQL

SEQ ID NO: 287 - protein, 9 aa, Homo sapiens
GSNINKSLK

SEQ ID NO: 288 - protein, 9 aa, Homo sapiens
TRDINKALY

SEQ ID NO: 289 - protein, 9 aa, Homo sapiens
ESFSIYVYK

SEQ ID NO: 290 - protein, 9 aa, Homo sapiens
ESYSIYVYK

SEQ ID NO: 291 - protein, 9 aa, Homo sapiens
KQSASAVHV

SEQ ID NO: 292 - protein, 9 aa, Homo sapiens
FNTASALHL

SEQ ID NO: 293 - protein, 9 aa, Homo sapiens
ASAASALHL

SEQ ID NO: 294 - protein, 9 aa, Homo sapiens
VHVPVSVAM

SEQ ID NO: 295 - protein, 9 aa, Homo sapiens
TGSPVSIAL

SEQ ID NO: 296 - protein, 9 aa, Homo sapiens
KMLRIVELY

SEQ ID NO: 297 - protein, 9 aa, Homo sapiens
YSLRIIDLI

SEQ ID NO: 298 - protein, 9 aa, Homo sapiens
GRIELYRVV

SEQ ID NO: 299 - protein, 9 aa, Homo sapiens
FMAELYRVL

SEQ ID NO: 300 - protein, 9 aa, Homo sapiens
SPEELYRVF

SEQ ID NO: 301 - protein, 9 aa, Homo sapiens
HRVELYKVL

SEQ ID NO: 302 - protein, 9 aa, Homo sapiens
RIFSSSYVA

SEQ ID NO: 303 - protein, 9 aa, Homo sapiens
VLLSSSFVY

SEQ ID NO: 304 - protein, 9 aa, Homo sapiens
SSYVAFISY

SEQ ID NO: 305 - protein, 9 aa, Homo sapiens
GRIVAFFSF

SEQ ID NO: 306 - protein, 9 aa, Homo sapiens
HIIPFQPQK

SEQ ID NO: 307 - protein, 9 aa, Homo sapiens
KLLPFNPQL

SEQ ID NO: 308 - protein, 9 aa, Homo sapiens
LRRTTDRKL

SEQ ID NO: 309 - protein, 9 aa, Homo sapiens
LRKTTEKKL

SEQ ID NO: 310 - protein, 9 aa, Homo sapiens
TNTDHLFTV

SEQ ID NO: 311 - protein, 9 aa, Homo sapiens
FLFDHLLTL

SEQ ID NO: 312 - protein, 9 aa, Homo sapiens
ALLDHLITH

SEQ ID NO: 313 - protein, 9 aa, Homo sapiens
GLLGVWTVL

SEQ ID NO: 314 - protein, 9 aa, Homo sapiens
TPAGVYTVF

SEQ ID NO: 315 - protein, 9 aa, Homo sapiens
LLGVWTVLL

SEQ ID NO: 316 - protein, 9 aa, Homo sapiens
METVWTILP

SEQ ID NO: 317 - protein, 9 aa, Homo sapiens
GVWTVLLLL

SEQ ID NO: 318 - protein, 9 aa, Homo sapiens
SAITVFLLF

SEQ ID NO: 319 - protein, 9 aa, Homo sapiens
APRTVLLLL

SEQ ID NO: 320 - protein, 9 aa, Homo sapiens
LHNVGLLGV

SEQ ID NO: 321 - protein, 9 aa, Homo sapiens
GLTVGLVGI

SEQ ID NO: 322 - protein, 9 aa, Homo sapiens
AVKVGLVGR

SEQ ID NO: 323 - protein, 9 aa, Homo sapiens
GLLGSWTVL

SEQ ID NO: 324 - protein, 9 aa, Homo sapiens
SAGGSFTVR

SEQ ID NO: 325 - protein, 9 aa, Homo sapiens
HMDGSFSVK

SEQ ID NO: 326 - protein, 9 aa, Homo sapiens
YIALLFGAK

SEQ ID NO: 327 - protein, 9 aa, Homo sapiens
APSLLYGAL

SEQ ID NO: 328 - protein, 9 aa, Homo sapiens
KQQLLIGAY

SEQ ID NO: 329 - protein, 9 aa, Homo sapiens
SVGQDLLLY

SEQ ID NO: 330 - protein, 9 aa, Homo sapiens
KLNQDVLLV

SEQ ID NO: 331 - protein, 9 aa, Homo sapiens
SLFSELSPV

SEQ ID NO: 332 - protein, 9 aa, Homo sapiens
STASELSPK

SEQ ID NO: 333 - protein, 9 aa, Homo sapiens
TVAPVSVPR

SEQ ID NO: 334 - protein, 9 aa, Homo sapiens
VVGPVSLPR

SEQ ID NO: 335 - protein, 9 aa, Homo sapiens
AVSSLALEI

SEQ ID NO: 336 - protein, 9 aa, Homo sapiens
SSASLAVEY

SEQ ID NO: 337 - protein, 12 aa, Homo sapiens
RPFKGYEGSLIK

SEQ ID NO: 338 - protein, 12 aa, Homo sapiens
RLFKGYEGSLIK

SEQ ID NO: 339 - protein, 10 aa, Homo sapiens
RPFKGYEGSL

SEQ ID NO: 340 - protein, 8 aa, Homo sapiens
RTKQTARK

SEQ ID NO: 341 - protein, 8 aa, Homo sapiens
RIKQTARK

SEQ ID NO: 342 - protein, 9 aa, Homo sapiens
ARTKQTARK

SEQ ID NO: 343 - protein, 9 aa, Homo sapiens
SSYQVLVLL

SEQ ID NO: 344 - protein, 9 aa, Homo sapiens
RRGQVLILL

SEQ ID NO: 345 - protein, 9 aa, Homo sapiens
ATGFQSMVI

SEQ ID NO: 346 - protein, 9 aa, Homo sapiens
FTNRFKIPI